(19) United States
(12) Patent Application Publication
(10) Pub. No.: US 2026/0216510 A1
(43) Pub. Date: Jul. 30, 2026

(54) TRANSCUTANEOUS AURICULAR VAGAL NERVE STIMULATION FOR PERI-INTERVENTION INFLAMMATORY MODULATION

(71) Applicant: Aurenar, Inc., St. Louis, MO (US)

(72) Inventors: Jesse Wheeler, St. Louis, MO (US); Eric Leuthardt, St. Louis, MO (US); Jenna Gorlewicz, St. Louis, MO (US)

(73) Assignee: Aurenar, Inc., St. Louis, MO (US)

(21) Appl. No.: 19/578,541

(22) Filed: Mar. 25, 2026

Related U.S. Application Data
(63) Continuation-in-part of application No. 19/355,071, filed on Oct. 10, 2025.
Application No. 19/355,071 is a continuation-in-part of application No. 19/186,253, filed on Apr. 22, 2025, now Pat. No. 12,470,859.
(60) Provisional application No. 63/698,299, filed on Sep. 24, 2024.
(60) Provisional application No. 63/713,773, filed on Oct. 30, 2024.
(60) Provisional application No. 63/744,537, filed on Jan. 13, 2025.

(51) Int. Cl. A61N 1/36; A61N 1/04

(52) U.S. Cl. CPC A61N 1/36021; A61N 1/0456; A61N 1/36031; A61N 1/36034; A61N 1/36036

(57) ABSTRACT

Methods and apparatuses for treating inflammatory diseases by neurostimulation in patients who have failed to adequately respond or have become intolerant to a drug therapy (such as a TNF inhibitor and/or a JAK inhibitor).

### CROSS REFERENCE TO RELATED APPLICATIONS

The present application is a continuation-in-part of U.S. application Ser. No. 19/355,071 filed Oct. 10, 2025, titled, ADJUSTABLE EAR WORN APPARATUS, which is a continuation-in-part of U.S. application Ser. No. 19/186,253, filed Apr. 22, 2025, titled ADJUSTABLE EAR WORN APPARATUS, now U.S. Pat. No. 12,470,859 issued Nov. 11, 2025, which claims the benefit of and priority to U.S. Provisional Patent Application No. 63/698,299, filed Sep. 24, 2024, titled ANTI-INFLAMMATORY AURICULAR VAGAL NERVE STIMULATION, U.S. Provisional Patent Application No. 63/713,773, filed Oct. 30, 2024, titled AURICULAR VAGAL NERVE STIMULATION TO MITIGATE INFLAMMATORY COMPLICATIONS FOR BIOACTIVE AGENTS, and U.S. Provisional Patent Application No. 63/744,537, filed Jan. 13, 2025, titled WEARABLE DEVICES WITH ADJUSTMENT MECHANISMS, the disclosures of which are incorporated herein by reference.

### BACKGROUND

The vagus nerve is linked to several neural systems of the body that can affect a wide variety of conditions. Commercial systems are available using an implanted pulse generator and a lead extending to the left vagus nerve, and are used to treat epilepsy or depression, and to aid post-stroke rehabilitation as the patient relearns aspects of physical movement. New and alternative ways of stimulating the vagus nerve are also being researched and written about. Some research suggests that the auricular branch of the vagus nerve may be usefully stimulated to address a range of conditions including stress, inflammation, mental focus, etc. New and alternative devices and methods for stimulating the vagus nerve near the ear are desired.

### SUMMARY OF INVENTION

Medical interventions including surgical procedures, immunotherapies, and implantable devices frequently trigger inflammatory responses that can lead to serious complications. For example, surgical trauma activates innate immune responses leading to systemic inflammatory response syndrome (SIRS), while novel immunotherapies such as CAR-T cell therapy can trigger cytokine release syndrome (CRS). Similarly, introduction of foreign materials through implantable medical devices or contrast agents can provoke inflammatory cascades. These inflammatory complications represent a significant clinical burden, contributing to morbidity, mortality, extended hospital stays, and increased healthcare costs.

The vagus nerve plays a central role in modulating inflammation through the cholinergic anti-inflammatory pathway and other neuroimmune mechanisms. Transcutaneous auricular vagal nerve stimulation (taVNS) provides a non-invasive means of activating these anti-inflammatory pathways by delivering electrical stimulation to the auricular branch of the vagus nerve in the ear. However, existing approaches have not comprehensively addressed the application of taVNS in the peri-intervention period before, during, and after medical interventions to prevent or mitigate intervention-associated inflammatory complications. There remains a need for systems and methods that apply taVNS in a targeted, protocol-driven manner to address inflammatory complications across diverse categories of medical interventions, with personalized approaches based on patient risk factors and intervention characteristics.

Accordingly, the present disclosure provides methods, systems, and devices for mitigating inflammatory complications associated with medical interventions using transcutaneous auricular vagal nerve stimulation (taVNS). The approaches disclosed herein recognize that medical interventions can be broadly categorized based on their inflammatory mechanisms, and that taVNS can be applied in a targeted, phase-specific manner throughout the peri-intervention period to activate neuroimmune anti-inflammatory pathways.

In one aspect, a method of mitigating inflammatory complications associated with a medical intervention in a patient comprises applying transcutaneous auricular vagal nerve stimulation to the patient at one or more times selected from before the medical intervention, during the medical intervention, and after the medical intervention, wherein the transcutaneous auricular vagal nerve stimulation is delivered via one or more stimulation elements positioned on an auricle of the patient, and wherein the transcutaneous auricular vagal nerve stimulation activates one or more neuroimmune anti-inflammatory pathways to reduce inflammatory complications associated with the medical intervention.

The medical intervention may be selected from tissue-disruptive interventions (including surgical procedures, radiation therapy, and ischemia-reperfusion procedures), immune-activating interventions (including CAR-T cell therapy, immune checkpoint inhibitors, and gene therapies), or foreign-material-introducing interventions (including implantable devices, contrast agents, and transfusions). The inflammatory complications may include cytokine release syndrome, cerebral edema, systemic inflammatory response syndrome, acute kidney injury, immune effector cell-associated neurotoxicity syndrome, capillary leak syndrome, and ischemia-reperfusion injury, among others.

The neuroimmune anti-inflammatory pathways activated by taVNS may include cholinergic anti-inflammatory pathway activation, central network suppression, reduced cerebral metabolism, autonomic stabilization, sympathetic withdrawal, endothelial stabilization, thromboinflammation modulation, HPA-axis modulation, microglial phenotype shifting, gut-immune axis modulation, and pain-inflammation feedback loop interruption.

In various embodiments, the method includes patient risk stratification based on baseline inflammatory biomarkers, medical history, and intervention type, with protocol selection tailored to the patient's risk tier. Stimulation parameters may be adjusted based on real-time physiologic feedback including heart rate variability, blood pressure variability, and inflammatory biomarker levels. Multi-phase protocols may deliver different stimulation parameters before, during, and after the medical intervention based on phase-specific inflammatory modulation objectives.

In another aspect, a transcutaneous auricular vagal nerve stimulation device configured for peri-intervention inflammatory modulation comprises a housing adapted for placement relative to an ear of a patient, one or more stimulation elements positioned to contact auricular tissue, a controller coupled to a memory storing instructions, and output circuitry coupled to the controller and the one or more stimulation elements. The instructions, when executed, cause the output circuitry to deliver stimulation via the one or more stimulation elements according to a peri-intervention protocol selected based on an intervention category.

In a further aspect, a non-transitory computer-readable medium stores instructions that, when executed by a controller of a taVNS device, cause the device to receive at least one input selected from a medical intervention type, a patient risk stratification parameter, or both; select a peri-intervention stimulation protocol based on the input(s); and deliver stimulation according to the selected protocol to mitigate inflammatory complications.

This summary is intended to provide an overview of the subject matter of the present disclosure. It is not intended to provide an exclusive or exhaustive explanation. The detailed description is included to provide further information.

### BRIEF DESCRIPTION OF THE DRAWINGS

The drawings illustrate, by way of example, but not by way of limitation, various embodiments discussed herein. In the drawings, which are not necessarily drawn to scale, like numerals may describe similar components in different views.

FIG. 1 is a sketch of the human ear;

FIGS. 2A-2C are views of a first wearable device;

FIGS. 3A-3C are views of a second wearable device;

FIGS. 4A-4C are views of a third wearable device;

FIGS. 5A-5C are views of a fourth wearable device;

FIG. 6 illustrates placement of a wearable device;

FIG. 7 is a side section view illustrating speaker positioning in the ear;

FIG. 8 shows an illustrative example of status and warning lights;

FIG. 9 shows an illustrative example of status and warning lights;

FIG. 10 is a block diagram for illustrative circuitry;

FIG. 11 shows a block process flow diagram of an illustrative method;

FIGS. 12-13 illustrate wearable devices and charging systems;

FIGS. 14-15 illustrate further alternative designs for wearable devices;

FIG. 16 shows a block process flow diagram of an illustrative method for treating inflammation;

FIGS. 17A-17G show illustrative stimulation device designs;

FIG. 18 illustrates various electrode configurations;

FIG. 19 shows an intervention classification diagram;

FIG. 20 shows neuroimmune pathways (Central Mechanisms);

FIG. 21 shows neuroimmune pathways (Peripheral Mechanisms);

FIG. 22 shows a patient risk stratification flowchart;

FIG. 23 shows a parameter adjustment matrix;

FIG. 24 shows a closed-loop adaptive system diagram; and

FIG. 25 shows a peri-intervention inflammatory modulation framework illustrating three potential independent application tracks for tissue-disruptive, immune-activating, and foreign-material-introducing interventions.

### DETAILED DESCRIPTION

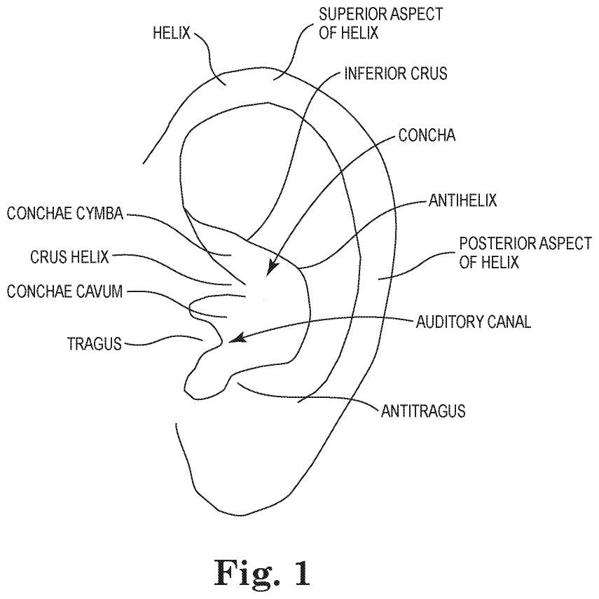

FIG. 1 is a sketch of the human ear. The auditory canal is covered at its opening by the tragus, and opens adjacent the concha. The concha is typically bisected by the crus helix into the conchae cymba superiorly and conchae cavum inferiorly. The helix is the outer rim of the ear that extends from the superior insertion of the ear on the scalp to the termination of the cartilage at the earlobe, having a superior aspect and posterior aspect, as marked in the drawing. The border of the helix usually forms a rolled rim, but the helix is highly variable in shape. The crus helix is the continuation of the anteroinferior ascending portion of the helix, and as shown in the drawing, extends in a posteroinferior direction into the cavity of the concha, typically about one half to two thirds the distance across the concha. The concha is generally bordered by the antihelix superiorly and antitragus inferiorly.

The aim in some examples herein is to provide a wearable device which is compact and non-intrusive, being easily placed and operated for a user. Some examples may have an intended life of up to two weeks or longer, after which the device is intended to be discarded. Alternatively, the device can be discarded after each individual use. Other examples may provide such a wearable device, but for use at home or in other contexts and for different time durations. Some examples are characterized by having the anchoring devices are all in a single housing, which may include a clip or may be used with adhesive tape for securing the apparatus in place.

Moreover, in some embodiments, the system and/or device herein may be provided with an audio output device (e.g., a transducer or speaker). For example, the transducer or other type of audio output element can be positioned in one or both of the extending structures described herein. Hence, in some embodiments an audio output device such as a speaker can be positioned on the first extending structure or the second extending structure. A circuit board, as described herein, can be configured to operate (e.g., turn on/off, alter volume, etc.) the transducer or other type of audio output element. For instance, the system/device herein may be manifested as an auditory ear bud including a transducer (with or without a sensor/stimulator element), such as a speaker.

While some figures herein are described as having electrodes as vagus nerve stimulation elements for purpose of delivering therapy to a user or patient, other devices, methods and/or modalities can be used. Examples may use any of optical stimulation with light sources (optical transducers) such as lasers (including vertical cavity emitting lasers) or light emitting diodes including, for example and without limitation, optical stimulation using wavelengths in the infrared, near-infrared, and/or visible spectrum. Other examples may use vibratory or acoustic stimulation with frequencies from relatively low levels (tens to hundreds of hertz) up to ultrasound frequency. Such stimulation may be described as mechanical stimulation, and may use a mechanical transducer to convert electrical energy to acoustic/vibratory energy using, for example a speaker or ultrasound generator. Some examples may use magnetic stimulation with electromagnetic fields generated using, for example, permanent magnets or electro-magnetic sources such as one or more inductive coils or other magnetic transducers.

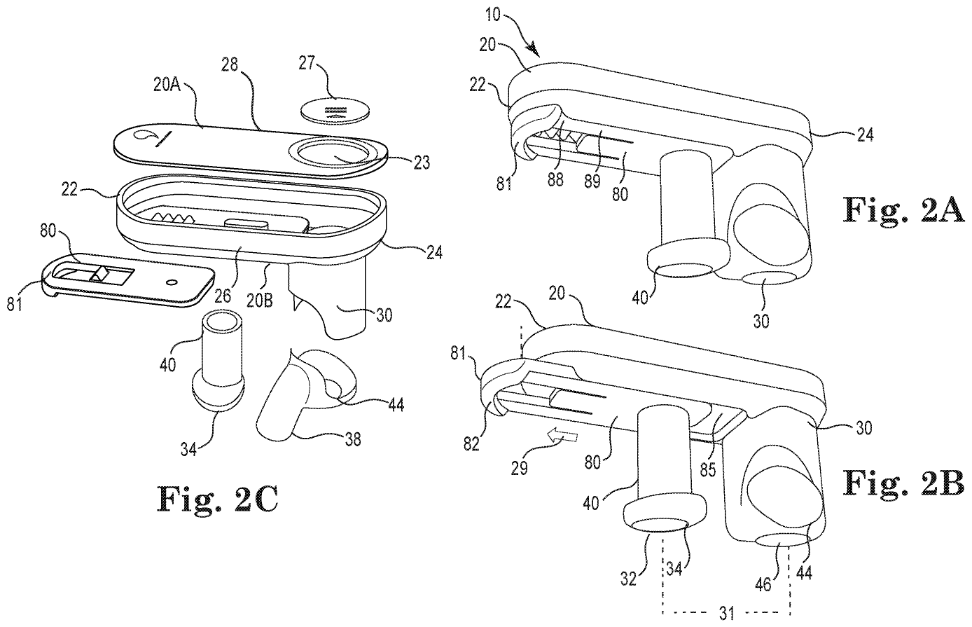

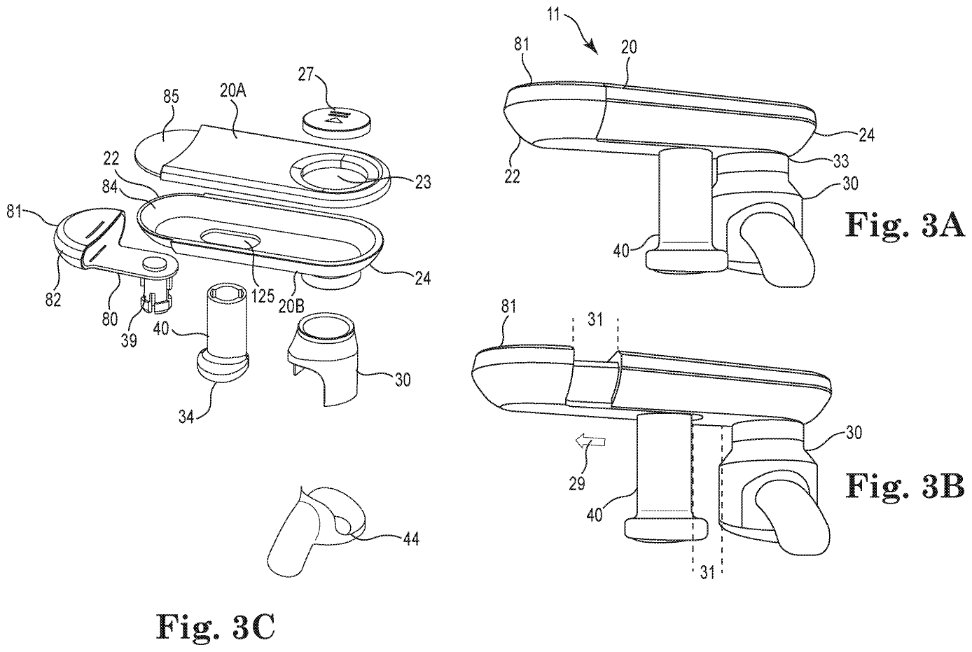

While the example of FIGS. 2A-2C (and similarly FIGS. 3A-3C, 4A-4C, and 5A-5C) is described as having speakers, in some embodiments sensors such as those described herein, other devices, methods and/or modalities can also be used. For instance, some examples herein may be directed to non-therapeutic, sensory, and/or purely auditory devices such as those including a speaker e.g., for the purpose of emitting sound or music to a user of the auditory devices and/or an electrical sensor. However, some examples may include a sensor such as electrical, optical, and magnetic sensors configured to detect or sense galvanic skin impedance, heart rate, heart rate variability, oxygenation, movement or acceleration (e.g., an accelerometer and/or gyroscope), various signals such as nerve and brain signals (e.g., an electroencephalograph), and/or the presence of one or more molecules (e.g., optical or chemical sensors) and/or configured to sense molecules in or on tissue, among other types of sensors. The sensor can be configured to detect or sense one or more of the above mentioned elements, statuses, actions, or characteristics while the devices herein are inserted in an ear of a user. As detailed herein, the sensor wherein a sensor can be positioned on the first extending structure or the second extending structure. In some embodiments, a speaker can be positioned on one of the first extending structure and the second extending structure and the sensor can be positioned on the other of the first extending structure and the second extending structure. However, in some embodiments the speaker and a sensor can each be located on either the first extending structure or the second extending structure (e.g., the speaker and sensor are collocated on an individual extending structure).

FIGS. 2A-2C are views of a first wearable device 10. FIGS. 2A-2B are views of the wearable device 10, while FIG. 2C shows an exploded view of the wearable device 10. The device 10 is adapted for placement relative to an ear of a user.

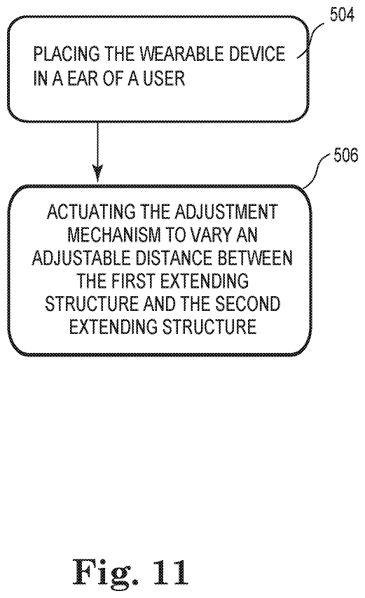

A housing 20 contains electronics and a power source configured for providing output energy, which may come in various forms including, in some examples, electrical pulses or other waveforms. In some embodiments, the electronics comprises an output circuitry to provide power at least to the speaker. Illustrative circuitry is shown and discussed relative to FIG. 10, below.

The housing 20 has a length between a first end 22 and a second end 24, and a width between a first side or edge 26 and a second side or edge 28. The housing can be an elongated housing where the length between a first end 22 and a second end 24 is greater than the width in this example. For example, the length may be in the range of about 10 to about 60 millimeters, and width in the range of about 3 to about 30 millimeters, or more or less. The overall mass of the device 10 may be in the range of about 10 to about 50 grams, or more or less.

The device 10 includes a first extending structure 30 having a first end at the housing 20 and a second end apart from the housing 20. The first end of the first extending structure 30 can by coupled to or proximate to a proximal end of the carriage 80, as illustrated in FIGS. 2A-2B.

The first extending structure 30 has a length, generally in the range of about 3 to about 15 millimeters or more or less. At or near the second end of the first extending structure 30 is an anchor arm 44 extending laterally therefrom. In some examples, the device 10 may be characterized by the anchor arm 44 being configured to be positioned beneath the tragus when the device is placed. In some further examples, the anchor arm 44 is configured to be inserted into the external auditory canal of the user, providing at least a first anchoring point for the device.

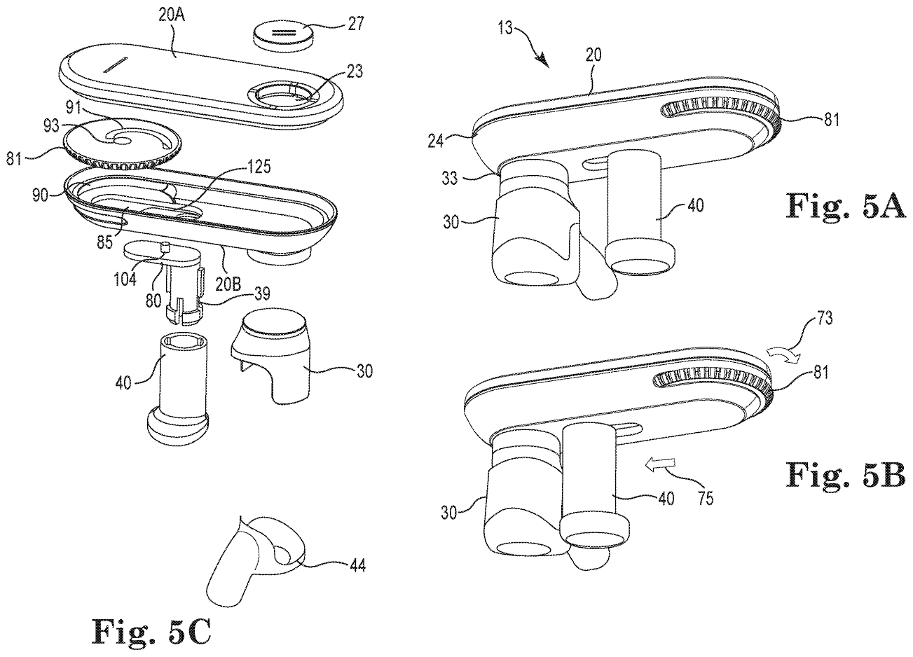

The first extending structure 30 can include a neck portion 33 located along the length of the first extending structure 30. For instance, the neck portion 33 can be located proximate or adjacent to the housing 20, as illustrated in FIGS. 3A and 5A. The neck portion 33 can have a smaller diameter than another portion or the remainder of the first extending structure 30. Stated differently, the neck portion 33 can form an indented portion of the first extending structure 30. In some embodiments, the neck portion 33 can be configured to provide an area into which tissue such as flaps of skin of the tragus and/or antitragus can overlay when the devices herein are inserted into an ear. For instance, the tragus and antitragus protrusions of the ear can desirably apply a force on the first extending structure 30 at the necked portion 33 such that the first extending structure 30 is securely retained in the ear (e.g., in the cavum) and/or an element located on the first extending structure 30 such as a speaker is thereby affixed firmly against the ear (e.g., the cavum). The neck portion 33 can optionally be configured to rotate relative to the housing 20 and/or another portion or the remainder of the first extending structure 30. The degree of rotation of the neck portion 33 can be at least 15 degrees, at least 30 degrees, or at least 45 degrees, among other possibilities.

The housing 20 includes or is attached to a second extending structure 40 having a first end at the housing 20 and a second end apart from the housing 20. The first end of the second extending structure 40 can be coupled to the carriage 80. For instance, the first end of the first extending structure 40 can be coupled to an outer surface such as the lowermost surface of the carriage 80, as illustrated in FIGS. 2A-2B. The second extending structure 40 has a length which may be in the range of about 3 to about 15 millimeters, or more or less. The length of the second extending structure may be variable, for instance, due to the presence of a spring structure or other biasing member or structure to allow flexibility or variability in a length of the housing 20 when the device is placed within an ear of a user.

A distance 31 between the first extending structure 30 and the second extending structure 40 can be adjusted. The distance 31 can be taken from a centerline or center point of each of the first extending structure 30 and the second extending structure 40, as illustrated in FIG. 2B or can be taken from opposing surfaces of the first extending structure 30 and the second extending structure 40, as illustrated in FIG. 3B. Hence, the distance 31 can refer to a distance between opposing faces of the respective first extending structure 30 and the second extending structure 40 and/or a distance between a central axis or respective center points of the first extending structure 30 and the second extending structure 40. The distance 31 can extend substantially along a longitudinal axis of the device 10. The distance 31 can be equal to a portion of the length of the device 10. The distance 31 can be adjustable or variable when the device 10 is placed within an ear of a user. For example, the distance 31 may be in the range of about 2 to about 20 millimeters, when the device is inserted in an ear of a user (e.g., when at least a portion of the first extending structure 30 and the second extending structure 40 are inserted in an ear of a user. The distance 31 can be varied by moving (e.g., longitudinally translating) the second extending structure 40 relative to the first extending structure 30. For instance, the first extending structure 30 may be fixed at a given longitudinal position and the second extending structure 40 may be configured to move (e.g., longitudinally translate distally relative to the first extend extending structure 30), as detailed herein. For instance, the second extending structure 40 may be configured to longitudinally translate responsive to actuation of an adjustment mechanism 81. Conversely, the first extending structure may be configured to longitudinally translate responsive to actuation of an adjustment mechanism 81, for example, when the first extending structure 30 is coupled to a proximal end of the carriage 80, and the second extending structure is coupled to the housing. Furthermore, both the first and second extending structures may be configured to longitudinally translate responsive to actuation by one or more adjustment mechanism 81 and carriages 80.

The adjustment mechanism 81, as detailed herein, can refer to an individual adjustment mechanism that is manifested as an individual component. Thus, the adjustment mechanism 81 can be configured to permit readily adjusting the distance 31, even when the device 10 is disposed in a user (e.g., an ear of a user). For instance, the adjustment mechanism 81 can be configured to permit a user (e.g., a user) to adjust the distance 31 while the device 10 is disposed within an ear of the user by actuation of the adjustment mechanism with a single hand (e.g., one or more digits on the single hand), as compared to some other devices which require any adjustment to be performed prior to insertion of a device in the user and/or which require the use of two or more hands to adjust the device. That is, the adjustment mechanism 81 permits precise adjustment of the second extending structure's position relative to the first extending structure's position, ensuring optimal placement of the speaker or audio output device(s) e.g., on the conchae cymba without the need of the user to remove the device from the ear.

The adjustment mechanism 81 can be located at the first end 22 of the housing 20. Having the adjustment mechanism 81 be located at the first end 22 of the housing can promote aspects herein such as permitting a user to readily adjust the distance 31, even when the device 10 is disposed in an ear or a user. For instance, the adjustment mechanism 81 can be located at the first end 22 of the housing 20 and a portion (e.g., first portion) of the adjustment mechanism 81 can be located outside of the housing 20, while another portion of the adjustment mechanism 81 can be located inside of the housing 20. The portion of the adjustment mechanism 81 that extends outside of the housing 20 can thus extend (e.g., in a substantially longitudinal direction) a distance away from the first end 22 of the housing 20. The portion of the adjustment mechanism 81 that extends outside of the housing 20 can be contacted by one or more digits on a hand of a user and the other portion of the adjustment mechanism can be configured to adjust or move the carriage 80 responsive to the contact. The portion (e.g., second portion) of the adjustment mechanism 81 that is inside the housing can be integral with or coupled to the carriage 80. For instance, as detailed herein, the second extending structure 40 can be coupled to the carriage 80. Thus, the movement of the carriage 80 can impart a corresponding movement in the second extending structure 40. For example, actuation of the first portion of the actuation mechanism 81 can directly or indirectly cause the carriage 80 to move, as detailed herein, thereby imparting a corresponding movement in the second extending structure 40 (e.g., in the same direction and magnitude as the movement in the carriage 80). One or more mechanisms 81 can be integral or coupled to more than one carriage 80 to impart movement on both the first and second extending structures.

As mentioned, in some embodiments the adjustment mechanism 81 can be integral with the carriage 80. For example, as illustrated in FIGS. 2A-2C and 3A-3C, the adjustment mechanism can be integral with the carriage 80. In such examples, the adjustment mechanism 81 can be located on a first end of the carriage 80. The first end of the carriage 80 refers to an end of the carriage 80 that is spaced away or least proximate to the first and second extending structures. Having the adjustment mechanism 81 be located on or integrally formed in the first end of the carriage 80 can promote aspects herein such as readily permitting a user to adjust the distance 31, even when the device 10 is implanted or disposed in an ear of a user. For instance, the adjustment mechanism 81 can include a fixed protrusion extending radially from the carriage 80. Stated differently, the adjustment mechanism 81 can be formed of an integral fixed protrusion that extends from the carriage 80. For example, the adjustment mechanism 81 can be manifested as a raised surface or protrusion that extends (e.g., radially and/or longitudinally) a distance from the carriage 80, as illustrated in FIGS. 2A-2C. Other configurations of the adjustment mechanism 81 are possible. For instance, the adjustment mechanism 81 can be manifested as a recessed or indented portion of a surface (e.g., an indented portion in an end of a protrusion extending radially from the carriage 80), as illustrated in FIGS. 3A-3C.

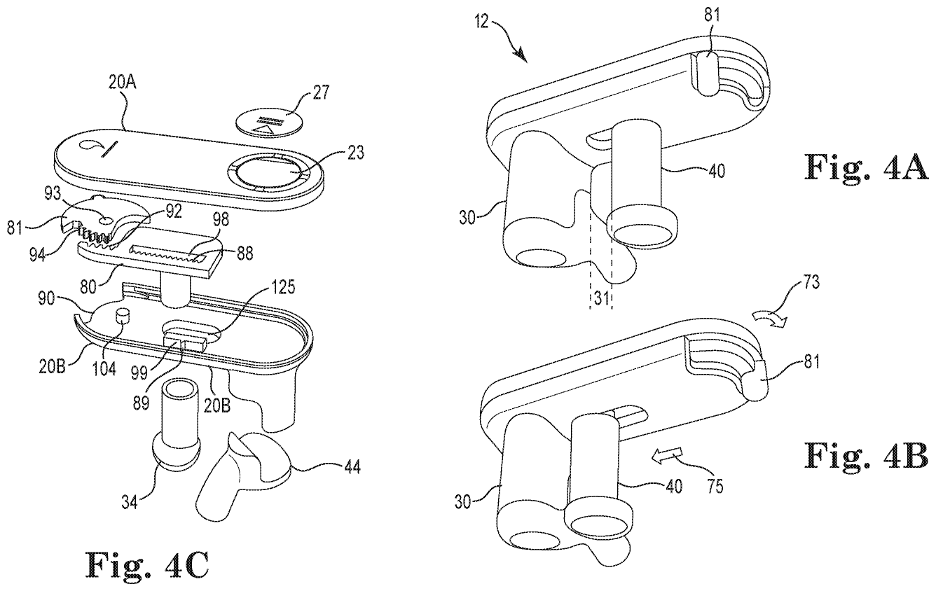

As mentioned, in some embodiments the adjustment mechanism 81 can be a separate component that is coupled to the carriage 80. For instance, the adjustment mechanism 81 can be a separate component that is coupled to a first end (e.g., that is spaced away from or least proximate to the first and second extending structures) of the carriage 80. For example, as illustrated in FIGS. 4A-4C and 5A-5C, the adjustment mechanism 81 can be a separate component that is coupled to the first end of the carriage 80. In such instances, the adjustment mechanism 81 can be a rotatable adjustment mechanism such as a rotatable wheel or rotatable lever. For instance, the adjustment mechanism 81 can be a rotatable adjustment mechanism including an annulus (e.g., annulus 93, as illustrated in FIGS. 4C and 5C), wherein the rotatable adjustment mechanism is configured to rotate about the annulus 93 relative to the carriage 80, the housing 20, or both the carriage 80 and the housing 20, as detailed herein. Hence, the rotatable adjustment mechanism (e.g., a rotatable lever or rotatable wheel) can be configured to rotate about a plane (extending through the annulus 93) that is normal to a longitudinal axis of the device 10 such that rotation of the adjustment mechanism imparts a force to cause longitudinal movement (e.g., translation) of the carriage 80, as detailed herein. In some embodiments, the carriage 80, the actuation mechanism 81, and/or the housing 20 can include detents that are configured to predispose the carriage 80 to corresponding longitudinal positions along a length of travel (e.g., longitudinal translation) of the carriage 80. Employing detents can promote aspects herein such as promoting retention of the devices herein within an ear of a user i.e., once the carriage 80 is disposed at a given longitudinal position corresponding to one or more of the detents. For instance, the presence of the detents can permit the carriage 80 to move between and be disposed in one or more fixed positions (e.g., three different longitudinal positions) to accommodate different sized ears (e.g., small, medium, and large sized ears) of various users of the devices herein.

The detents can be mechanical detents and/or magnetic detents (e.g., formed of two or more magnets including a magnet coupled to the carriage 80 and a magnet coupled to the housing 20). For instance, the carriage 80 can include detents located along one or more of the substantially longitudinally extending side surfaces of the carriage 80 and/or that are located along the elongated slot 98 or other aperture in the carriage 80. In some embodiments, the detents of the carriage 80 can be manifested as one or more substantially radially projecting arms or features. In some embodiments, the detents of the carriage 80 can be manifested as a series of undulating ridges (e.g., peaks and valleys disposed therebetween). The detents can be configured to predispose the carriage 80 to longitudinal positions associated with the valleys (e.g., at spaced intervals between adjacent valleys), while the peaks can be configured to provide a degree of resistance to the longitudinal movement (e.g., translation) of the carriage 80. The housing 20 can include one or more corresponding detents or projections that are configured to mechanically interfaces with the detents of the carriage 80. In some embodiments, the corresponding detents 89 can be manifested as one or more arms or projections or as a series of undulating ridges (e.g., peaks and valleys). In some embodiments, the corresponding detents 89 can be manifested as one or more pegs or projections, as illustrated in FIG. 4C.

As detailed herein, the carriage 80, the housing 20, or both the carriage 80 and the housing 20 can include detents that are configured to predispose the carriage to one or more longitudinal positions along a length of travel of the carriage 80. For example, FIG. 4C illustrates the presence of detents 88 along a substantially longitudinally extending surface of the elongated slot 98. In such instances, the housing 20 can have a corresponding substantially longitudinally extending surface with corresponding detents. For example, an interior surface (most proximal to the carriage 80) of an elongated peg 99 can include detents 89 that are configured to interface with (e.g., interference fit with) the detents 88 and permit the carriage 80 to be moved longitudinally between one or more positions at which the detents 88 of the carriage 80 and the corresponding detents 89 of the housing 20 predispose the carriage 80.

In some embodiments, the location of the detents 88 of the carriage 80 and/or the location of the corresponding detents of the housing 20 can be varied for the locations in FIG. 4C. For instance, the detents 89 of the housing 20 can be located on one or both of the molded pieces 20A, 20B. In other examples, the detents can be located on the adjustment mechanism 81, the housing 20, or both the adjustment mechanism 81 and the housing 20. For example, detents 88 can be located on the adjustment mechanism 81 along at least a portion of an exterior surface of the adjustment mechanism 81 and the housing 20 can include corresponding detents 89 that are configured to interface with the detents 88 on the actuation mechanism 81. An example of alternative locations for the detents 88 and the corresponding detents 89, is indicated in FIG. 2A.

Alternatively, or in addition to varying the location of the detents 88 and/or the corresponding detents 89, the structures of the detents 88 and/or the corresponding detents 89 can be varied. For instance, one of the detents 88 and the corresponding detents 89 can be manifested as a peg or projection, while the other of the detents 88 and the corresponding detents 89 can be manifested as a series of undulating ridges (e.g., peaks and valleys). For example, the detents 88 can be configured as a series of undulating ridges and the corresponding detents 89 can be manifested as one or more peg or projection that is configured to interface with the detents 88, as illustrated in FIG. 4C, or the detents 88 can be configured as a peg or projection and the corresponding detents 89 can be manifested as a series of ridges (e.g., peaks and valleys).

The housing 20 may comprise molded pieces 20A, 20B assembled together. In this example, the first molded piece 20A may be an upper lid and a second molded piece 20B may have a lower container to which the upper lid attaches, collectively forming housing 20. In this example, the first extending structure 30 is shown to be integrally molded or otherwise formed as an integral part of the lower container 20B or another portion of the housing 20. In other embodiments, the second extending structure 40 can be integrally molded or otherwise formed as an integral part of with the lower container 20B or another portion of the housing 20. Molded into the lower container 20B is an internal channel 85 configured to receive carriage 80. However, other configurations of the lower container 20B and the carriage 80 such as those having the internal channel 85 in a different location or an absence of the internal channel 85 are possible. In some examples, the carriage 80 and the upper portion (most proximate to the carriage) of the second extending structure 40 may be molded together as a singular piece for positioning into the internal channel 85.

In some embodiments, the lower container 20B also includes an elongated aperture 125, situated between the first extending structure 30 and the first end 22 of the housing 20, for instance, as illustrated in FIGS. 3C, 4C, and 5C herein. In such embodiments, the carriage 80 can include a lower portion the extends through the aperture 125 to contact an upper portion of the second extending structure 40. Alternatively, an upper portion of second extending structure 40 can protrude through the aperture 125 away from the housing 20. In any case, the position of the carriage 80 in housing 20 (e.g., the position of the carriage 80 within the internal channel 85) is adjustable, and by virtue of the second extending structure 40 being coupled to carriage 80, the position of the second extending structure 30 is also adjustable. That is, the space between the first extending structure and the second extending structure is adjustable.

In some embodiments, the aperture 125 allows the second extending structure 40 to protrude through the lower container 20B and the internal channel 85 exceeds the length of the aperture 125, providing a guided pathway for the carriage 80 to slide within the lower container 20B. As a consequence of such configuration, the range of the carriage 80 movement can be limited to the extent that the second extending structure 40 can moveably slide within the bounds of the elongated aperture 125. This range of movement is illustrated, for instance, in part by the arrow 29 in FIG. 3B. By limiting such movement to the range allowed by the elongated aperture 125, manufacturing and assembly are made easier than if the housing had two components which slide together and apart, as controlling the maximum extent of movement is relatively simple. In an alternative example, rather than an aperture as shown in FIG. 3B, the overall housing 20 may have first and second components that mate together in sliding fashion to allow the length of the housing itself to be varied.

In some examples, a shroud or cover can overlay a portion of or an entirety of the apertures described herein. The shroud or cover can be configured to prevent or mitigate ingress of material (e.g., liquids and/or particulate materials). The cover can be formed of a relatively thin and/or deformable sheet of material. Examples of suitable materials for the cover include various thermoplastics (e.g., polyethylene, polypropylene, polyvinyl chloride, Polyethylene Terephthalate, EVA (Ethylene Vinyl Acetate), Polyamide (Nylon), among others. In some examples, the cover can be located internal to the housing 20 and permit a component such as at least a portion of the carriage 80 to project through the cover to a location outside of the housing 80, for instance, to mitigate ingress of material into the housing 20.

During or subsequent to placement of the device in the ear of the user, a position (longitudinal position) of the carriage 80 can be adjusted such that the sensor 32 is positioned on a conchae cymba while the first electrode or speaker 46 is positioned desirably at the conchae caverna. Yet, in some examples, the second electrode or sensor 32 may be positioned at the conchae caverna. In this configuration, the second electrode or sensor 32 is not part of the second extending structure 40, but rather integrated into housing 20 or the first extending structure 30, permitting the second electrode or sensor 32 to be in contact with different areas of the conchae once the device 10 is in position. In this example, the second extending structure 40 remains useful for stabilizing and securely attaching the wearable device 10 to the ear, regardless of whether a sensor or speaker is carried thereon.

In some examples, the user may adjust the positioning of the second electrode or sensor 32 by manipulating a position of the carriage 80 with the housing 20 from outside of the device e.g., wearable device 10. By facilitating adjustment from outside of the wearable device 10, the wearable device 10 does not have to be removed from its previously secured location (e.g., within an ear). This form of manipulation also permits precise adaptation to individual ear anatomies, without compromising the stability of the wearable device 10 placement as previously positioned. Adjustment of the carriage 80 in the housing 20 (e.g., within the internal channel 85 in the housing 20) may be done manually or via a spring-loaded mechanism. In the examples that use a spring-loaded mechanism, the mechanism can be compressed during wearable device 10 placement in the ear and subsequently released to expand relative to a portion of the ear (e.g., the inferior crus, helix, or antihelix), effectively securing the wearable device 10 in the ear. This expansion creates a counterforce against the conchae cavum, so that the first extending structure 30 is in contact against tragus and/or anti-tragus, ensuring a snug fit. In some examples, the spring-loaded mechanism incorporates a latch system, allowing the wearable device 10 to be locked in various positions between fully compressed and fully extended states. This allows further customization the fit and positioning of the wearable device 10 in accordance to the user's ear anatomy and comfort preferences, while maintaining the device's stability and effectiveness. In the manual configuration, users may adjust the positioning as needed including, but not limited to, using their fingers, tabs, hooks, loops, or pivoting levers. This adjustment can be done either before or after wearable device 10 has been placed in the ear.

The second extending structure 40 may have a variable shape, allowing for bending to a desired angle, and/or may rotate or pivot, so that the device can be adjusted to fit the user's ear.

As mentioned, in some examples, the housing 20 comprises molded pieces assembled together. For example, a first molded piece 20A may be an upper lid and a second molded 20B piece may be a lower container to which the upper lid attaches, thereby substantially forming the housing 20. Other manufacturing methods can be used. The first extending structure 30 may be a molded part of a lower container forming part of the housing 20, with the anchor arm 44 or a portion thereof included as part of the molding step, or attached thereto in a subsequent manufacturing step. Other assembly or manufacturing methods can be used.

The example shown here includes a first electrode or a speaker 46 on the first extending structure 30, and, optionally, a second electrode or sensor 32 on the second extending structure 40. There may be more than one electrode or element in each of these locations. Some examples may omit the second electrode or sensor 32 and/or may omit the first electrode or speaker 46. Rather than electrodes at 32, 46, devices for creating other therapy outputs (transducers, for example, for optical, mechanical/vibratory, magnetic, thermal or other therapies) may be used, in which case at least one transducer may be positioned on the first extending structure 30 and/or the second extending structure 40. Desirably, the positioning and/or degree of insertion of the anchor arm 34 may be such that the second electrode or sensor 32 and the first electrode or speaker 46 come into contact with the skin in the ear of the user.

The anchor arm, or “wing” 34, located at the second end of the second extending structure 40 may have an expanded end portion coupled by a thinner portion coupled to the second extending structure. Stated another way, the radial dimensions of the anchor arm or wing 34 may extend outward beyond (e.g., be larger than) the radial dimensions of the extending structure 40, as shown for example by FIG. 2B. The radial dimensions of the anchor arm or wing 34 may vary around its perimeter to form one or more edges or “lips” of varying size. The one or more ridges or lips of the anchor arm or wing 34 may be placed under the ridges and folds of skin formed by the helix, inferior crus, and antihelix. In some examples, when an illustrative device (e.g., the device 10) is placed in the ear, a portion of the anchor arm or wing 34 proximal to the helix may extend further than a portion of the anchor arm or wing 34 proximal to the inferior crus and antihelix, as appropriate to fit within the depths of skin folds formed by the helix, inferior crus, and antihelix. The curvature of the perimeter of the anchor arm or wing 34 may vary to optimally fit the curvature of the outer boundaries of the cymba formed by the helix, inferior crus, and antihelix. In some examples, the anchor arm or wing 34 may be rigidly coupled to the second extending structure 40. In some examples, the anchor arm or wing 34 may rotate relative to the housing 20. Rotation of the anchor arm or wing 34 may permit an improved fit to varying curvatures or differing geometries of the helix, inferior crus, and antihelix across users of the devices herein. The device 10 can be configured such that a distance between the first extending structure 30 and the second extending structure 40 is adjustable. For instance, adjustment of the distance between the between the first extending structure 30 and the second extending structure 40 can be adjustable via actuation or movement of the carriage 80. In such instances, movement of the carriage 80 can impart a corresponding movement (e.g., of the same magnitude and direction) in the second extending structure 40 and anchor arm or “wing” 34 e.g., as the anchor arm 34 is coupled to the first extending structure 40, which is coupled to the carriage 80. The device 10 can be configured such that the anchor arm or “wing”, 34, is positioned under any portion of the helix, inferior crus, and antihelix such that any portion of a ridge, or lip, of the anchor arm or wing 34 is held against the ear and resists dislodgement of the device 10 when a counter force is imparted against the tragus and antitragus when the carriage 80 is extended.

As highlighted in FIG. 2C, the anchor arm 44 may include an expanded or bulbous end portion coupled by a thinner portion to the first extending structure 30. While a bulbous end portion is shown, other shapes (oval, polygon, tapered, conical, etc.) may be used instead, and/or the end portion can be or include a foam material that can be compressed prior to placement, and then expands to secure the device in an anchored position. Alternatively, in other examples, the anchor arm 44 may have a consistent or tapered outer profile from its connection to the first extending structure 30 to its tip. The anchor arm 44 and end structure 38 may be a unitary or single piece, and may be hollow to allow audio signals to pass therethrough. In some examples, a speaker may be integrated into the device to deliver audio signals. The anchor arms herein such as the anchor arm 44 may also include one or more electrodes, sensors, and/or a speaker thereon. The anchor arm 44 and/or end structure 38 may be provided as a detachable/replaceable piece that can be selected from a range of sizes or shapes. In some examples, the anchor arm 44 and/or end structure 38 thereof may be formed of a compliant material to conform to the space under the tragus and/or inside the auditory canal.

The anchor arm 44 may extend at an angle relative direction of the length of the housing. The angle can be about ninety degrees, but in other examples it is envisioned that the angle can be in the range of about 60 to about 120 degrees, or about 70 to about 110 degrees, or about 80 to about 100 degrees. In an example, the angle of the anchor arm 44 may be adjustable, if desired, such as by use of a click-mechanism or flexible material to allow the anchor arm 44 to twist about the first extending structure 30. In still another example, the first extending structure 30 may be adjustable to twist about, for example, a central core (e.g., necked portion 33), entirely or through a limited range of motion such as (using the angle of the anchor arm as a guide) between about 60 to about 120 degrees, or more or less as desired. In the illustrative example shown in FIGS. 2A-2C, 3A-3C, 4A-4C, and 5A-5C, the position of the anchor arm 44 is fixed. One or more stops may be included to limit the translation, extension or rotation of the anchor arm 44, to the extent it is adjustable. The first and second extending structures 30, 40 may likewise be adjustable in terms of translation, extension/retraction and/or rotation, as desired.

If desired, one or more through-openings or holes may be provided in the anchor arm 44 to allow air ingress/egress, facilitating hearing for the user by avoiding complete blockage of the auditory canal. The anchor arm 44 may further include one or more electrodes, sensors and/or transducers, either for therapy purposes or to permit or augment hearing of a user or to emit music. For example, a speaker may be provided, allowing the user/user to hear audible indications of device and/or therapy status, to amplify sounds (as with a hearing aid), or to provide entertainment or communications to the user/user.

In first example, when the device 10 is placed relative to the ear of the user, the anchor arm 44 is positioned to extend beneath the tragus, while the second electrode or sensor 32 is positioned at (i.e. in contact with) the conchae cymba, and the first electrode or speaker 46 is positioned at (i.e., in contact with) the conchae caverna. In second example, when the device 10 is placed relative to the ear of the user, the anchor arm 44 is positioned to extend beneath the tragus, while the second electrode or sensor 32 and the first electrode or speaker 46 are on opposing sides of the crus helix. In a third example, when the device 10 is placed relative to the ear of the user, the anchor arm 44 is positioned to extend into the auditory canal, while the second electrode or sensor 32 is positioned at (i.e., in contact with) the conchae cymba, and the first electrode or speaker 46 is positioned at (i.e. in contact with) the conchae caverna. In an example, when the device 10 is placed relative to the ear of the user, the anchor arm 44 is positioned to extend into the auditory canal, while the second electrode or sensor 32 and the first electrode speaker 46 are on opposing sides of the crus helix. For instance, In an example, when the device 10 is placed relative to the ear of the patient, the anchor arm 44 is positioned to extend into the auditory canal, while the first and second electrodes are on opposing sides of the crus helix. These examples are not intended to be an exhaustive list of descriptions of the device positioning.

The electrodes herein may each have a surface area in the range of about 20 mm² to about 100 mm², or more or less. In some examples, each electrode as an area in the range of about 25 mm² to about 80 mm². The first electrode (e.g., element 46) may be larger than the second electrode (e.g., element 32) in some examples, allowing stimulation to be more targeted to the region of the second electrode by increasing the current density in the vicinity of the second electrode. The space or gap (edge to edge) between the electrodes may be in the range of about 2 mm to about 15 mm, or more or less. For example, electrodes may be about 5 mm to about 10 mm apart (edge to edge). Voltage and/or current controlled output waveforms may be used, as further described below. In some embodiments, the electrodes herein (e.g., the electrodes 32, 46) may be textured for instance to etching, scoring, pitted, porous, or comprised of one or more partially compressed strands. Employing textured electrodes (with a textured surface) can increase the surface area of the electrodes, thereby, decreasing interfacial impedance with the skin or interference through an applied conductive gel.

In some embodiments, the device 10 can be affixed to the ear in an absence of a clip, tape, and/or another type of attachment mechanism. For instance, as illustrated in FIGS. 2A-2C, the device 10 does not include a clip or adhesive tape. Instead, the device can be secured in place by the extending structures themselves and the anchor arms 44 and 34. The second extending structure 40, in some embodiments, aids in holding the device by having a variable length, using, for example, a spring loaded or otherwise adjustable connection between the upper part and lower part thereof, holding the second electrode or sensor 32 on the skin of the user while the anchor arm (or “wing”) 34 of the second extending structure 40 holds the device in a desired position and secures the placement of the second electrode or sensor 32. For instance, abutment of the second extending structure to a first portion of an ear (e.g., the inferior crus and antihelix) creates an opposing force to the first extending structure positioned against a second portion of the ear (e.g., the antitragus). Stated differently, in some embodiments the second extending structure is configured to abut a first portion of an ear to create an opposing force to the first extending structure which is configured to abut a second portion of the ear, wherein the first portion of the ear is the inferior crus, the antihelix, or both, and wherein the second portion of the ear is the antitragus. As detailed herein, the speaker, electrode, and/or a sensor can be located on the first extending structure and/or the second extending structure. Hence, the speaker, electrode, vibrating element, and/or sensor located on one or both of the first and second extending structures can readily function in various manners such as to convey sound to the ear of the user, deliver stimulation (e.g., electrical stimulation), and/or sense one or more parameters of conditions of the ear e.g., while the second extending structure abuts the first portion of the ear to create the opposing force to the first extending structure which abuts a second portion of the ear. Additionally, in some embodiment abutment of the anchor arm or wing of the second extending structure in the ear (e.g., beneath the anterior fold of the helix superior to the tragus) creates an opposing force to posterior rotation of the device relative to the ear, as does abutment of a portion of the anchor arm or wing of the second extending structure in the ear (e.g., beneath the fold of the inferior crus and/or antihelix). Optionally, the examples herein can also include a clip (not illustrated), adhesive tape, or other for clipping the device 10 into a desired position in the ear of a user.

Thermal stimulation may include heating of the nerve; heating may be achieved either by issuing higher frequency signals (RF heating), or by the use of a resistive heating element, for example and without limitation, wherein the resistive heating element may serve as a thermal transducer. Cooling may be provided, such as by having a removeable/replaceable thermal element that can be placed in a refrigerator prior to use, by including a Peltier cooling apparatus, or by having channels allowing cooling fluid to be circulated, either of which may be a thermal transducer. Thus, rather than the electrodes described above, one or more transducers can be used to convert stored power (usually electrical power from a battery) to a different energy modality. Each of these methods offers unique advantages and may be tailored to specific applications based on factors such as precision, invasiveness, and compatibility with the nerve tissue. For example, optical stimulation offers precise control over the timing and location of nerve activation. Acoustic and magnetic stimulation techniques can penetrate deeper tissues and may be non-invasive, making them suitable for certain clinical scenarios. Thermal stimulation, on the other hand, can modulate nerve activity by altering temperature gradients within the tissue.

In some examples, a combination of modalities can be used. For example, thermal stimulation may be generated by the use of higher frequency (RF) outputs from electrodes, paired with lower frequency pulsed electrical field outputs at frequencies in the tens to hundreds of hertz. Such signal combinations may be delivered in an overlapping or simultaneous manner, or the device may cycle between one therapy mode and another, as desired. Electrical stimulation can also be paired with magnetic, acoustic/vibratory (oscillating), and/or optical stimulation. Other combinations can be used as well.

Separate therapy may also be provided, such as with the delivery of anti-inflammatory or other medications to the patient along with the issuance of stimulation signals, or by also providing circulatory or respiratory support to the patient and/or additional stimulation signals, or thermal controls such as inducing therapeutic hypothermia or other temperature management. In some examples, therapy combined with an analgesic to ensure that the patient will not feel the therapy delivered by the stimulation device. An analgesic may be systemically delivered (injection, oral, etc.) or may be locally delivered such as by elution from the electrode surfaces or by using a gel or liquid containing analgesic substances (such as lidocaine) on the electrode surfaces.

The device may coordinate therapy delivery with other actions. In some examples, the device may be commanded to start a therapy session, while another therapeutic activity is ongoing, such as having the patient engage in a memory game or other activity while therapy is being delivered. Coordinated timing can be facilitated by use of the controls on the device itself, or the device may include communications circuitry (such as a Bluetooth or Bluetooth Low Energy antenna and chip) to communicate with a programming device or smartphone having counterpart communications circuitry; an application operating on the programming device or smartphone can be used to start therapy at a desired time and/or otherwise operate the devices herein (e.g., to cause a speaker in the device to emit sounds or music). Other coordination may include the use of biological signals. Heart rate, for example, can be monitored by the device itself (such as by adding or including an earlobe clip), or by a second device such as a cardiac monitor; when the heart rate is above a threshold, such as a threshold in the range of 100 to 140 beats per minute (or other setting), the patient may be experiencing a high degree of inflammatory response, so therapy can be turned on in response to elevated heart rate. On the other hand, if the heart rate becomes bradycardic, such as below about 40 to 60 beats per minute, therapy may be stopped. In another example, pupillometry can be used to turn therapy on or off by obtaining an image of the eye, using a smartphone or other device having a camera, and modulating or turning therapy on or off in response to the results of such measurements. Synchronization to other therapies, including physical therapy, drug delivery, or any other intervention can be useful to augment the patient's response to other therapies by Vagus nerve stimulation.

One or more portions of the housing and/or the speaker and/or sensor elements of the device can be designed to be modular, allowing for easy customization and adaptability to individual user needs. This feature permits users to easily change out the speaker and/or sensor as necessary, providing a tailored device for each user. For instance, the modular design enhances the versatility and flexibility of the device, ensuring that it can be easily adjusted to accommodate different anatomic requirements. Modularity may be provided by, for example, providing aspects of the device housing and/or neural stimulation elements in the system in a range of sizes or types. For example, if electrodes are used to issue electrical stimuli, the electrodes may come in different sizes (surface areas) and/or shapes, which may be selected and/or replaced. Modularity may be provided by, for example, providing aspects of the device housing and/or speaker, electrode, or sensor elements in the system in a range of sizes or types. For example, if a speaker is used to emit sound waves, the speaker or a portion of the housing on or in which the speaker resides (e.g., an earbud) may come in different sizes (surface areas) and/or shapes, which may be selected and/or replaced. Aspects of the housing and the extending structures can also be adjustable or replaceable to accommodate different anatomies (larger or smaller ears), including, for example, pediatric sized systems for smaller ears. The system itself may come in a range of sizes, if desired.

The device may be controllable and/or programmable or reprogrammable, such as by plug-in-type attachment to a port located on the device, or by use of magnetic/inductive, wireless (RF, such as Bluetooth) communication, optical communication, or by having one or more buttons, dials, or other user-accessible controls accessible on the device. To this end, as discussed further below with reference to FIG. 10, a communications circuitry may be included in the device.

The materials used throughout may include any material suitable for skin contact for an extended period of time (hours, days or even weeks). For an electrical stimulation system, the electrodes, for example, may be made of any of graphene, titanium, nickel titanium (nitinol), platinum, platinum-iridium, gold, silver, stainless steel (including MP35N alloy) or any other metal or conductive polymer or other material that can be worn on the skin. Coating layer(s) may be provided to optimize tissue interface characteristics, as desired.

The electrodes herein may be configured to receive or carry thereon a conductive material, such as a gel, hydrogel, or other tissue interface component. Pads may be attached if desired. Alternatively, dry electrodes can be used, if desired. The other tissue contacting portions of the devices herein may be made of suitable plastics, silicone, etc. adapted for wear on the skin of a patient/user.

Biocompatible materials may be selected to enhance conduction of the therapy signal between the electrode or therapy generating element and the tissue (electrical conduction, mechanical conduction, optical transmission, etc.). Biocompatible materials may also be selected to enhance adhesion of the therapy generating element and the tissue. Biocompatible materials may also be selected to provide an analgesic effect to suppress perception of the therapy. All types of materials may also be combined into a single material. Materials may be attachably and detachably connected to the therapy-generating element. For example, hydrogel pads may be replaced. In cases of wet materials, one or more moisture barriers (e.g. metal foil) may be used for packaging and temporarily adhered over the material to preserve functionality for extended shelf life. In other examples, materials may be separately packaged within a preserving pouch, packet, or container and applied prior to use. In some cases, the material may include a barrier material or membrane that is removed prior to use. In some cases, the barrier material or membrane may include extensions, tabs, buttons, or other structures to aid in handling the material while attaching the material or removing the material from the device. For instance, in some cases the conduction-enhancing materials or conduction-enhancing pads may be removably coupled or non-removably coupled to the ear-worn devices herein. Examples of removable coupling mechanism include a snap, button, pin, hook and loop coupling systems, and magnets, among other possibilities. In some cases where an attachable conduction-enhancing material or pad is used, an electrode that is integrated with the ear-worn devices herein can take the form of an electrical connector of various geometry including geometry that is configured to at least in part directly contact skin and/or with geometry that is configured to indirectly provide electrical stimulation, via conduction-enhancing material or pad, to skin of a user. Examples of suitable electrode geometries include a disc geometry and a dome geometry, among other possible shapes. The housing 20 optionally includes indicator lights such as status indicator lights and/or alert lights. If desired, an electrode, vibrating element, sensor, and/or speaker may be included in or on the housing 20 as well and used for issuing audible alerts, instructions for use, device status, or other purposes such as for providing an audible signal for entertainment or relaxation purposes (playing music for example). The status indicator lights may be light emitting diodes (LEDs) or any other suitable light generator, as desired.

In some embodiments, the device 10 can include one optical indicator for providing an indication of a state of the wearable device. For instance, the at least one optical indicator can be manifested as a multi-purpose light-emitting diode configured in a ring 351, as detailed herein.

The devices herein such as can contain a transceiver, such as a Bluetooth chip and antenna, to permit communication with an external device such as a smartphone or tablet. Alternatively, or in addition, in some embodiments the devices herein can include a port configured to receive a cable or a cord that can communicatively couple the devices herein to another device (e.g., a smartphone) that is configured to operate the devices herein. Other communication means can be used, including optical, magnetic/inductive, vibratory, etc., as desired. Alternatively, one or more buttons on the device may be used to increase or decrease output amplitude, as desired; additional indicators on the device may be used to allow amplitude settings to be determined visually. Some systems may, on the other hand, be pre-programmed with limited or no therapy adjustments available.

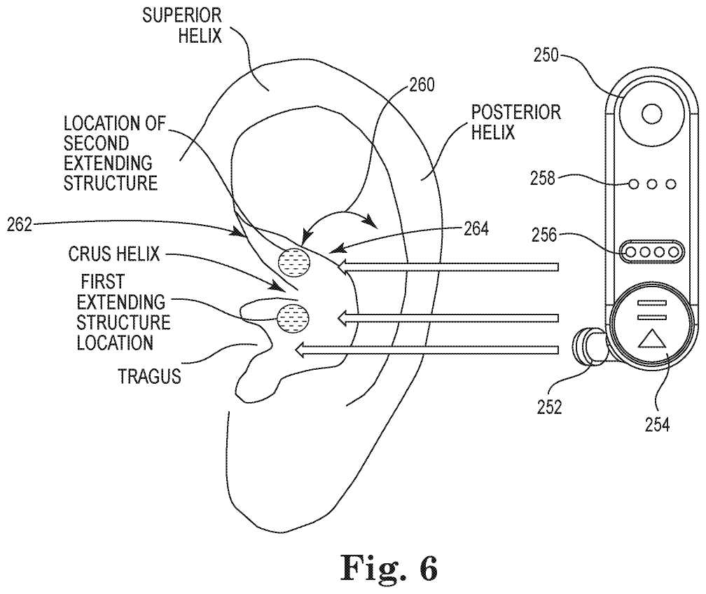

Some of the preceding examples indicate the use or possibility of reshapeable first extending structure 30 and/or second extending structure 40, or an anchor arm 44, or an anchor arm 34, which are reshapeable. Other examples make each of these pieces a rigid element not allowing for reshaping. In some examples, a rigid second extending structure 40 has a spring or other resilient member therein allowing the length to vary in response to user anatomy. The device may then be placed by inserting the anchor arm 44 with its end in the auditory canal of the user, and then twisting the device to bring the second extending structure into contact with the conchae cymba. The twisting movement may be as indicated by arrow and line 260 in FIG. 6, below, until the second extending structure 40 or attached anchor arm (or “wing”) 34 abuts the crus helix or antihelix, as explained relative to FIG. 6.

FIGS. 3A-3C are views of a second wearable device 11. The second wearable device 11 is analogous to the first wearable device 10 with the change that the adjustment mechanism 81 and components associated therewith (e.g., in contact therewith) are different. For instance, as illustrated in FIGS. 3A-3C, the adjustment mechanism 81 can include a protrusion 82 extending radially from the carriage 80 in a second direction (toward the first molded piece 20A), whereas the adjustment mechanism 81 of the first wearable device 10 is a protrusion 82 extending radially from the carriage in a first direct (away from the first molded piece 20A). Moreover, the adjustment mechanism 81 of the carriage 80 can be the first end 22 of the second wearable device 11, whereas the first end 22 of device 10 can be formed of the housing 20 (e.g., the carriage can move distal to the first end 22 of the device 10). Additionally, the protrusion forming the adjustment mechanism 81 of the second wearable device 11 can have an indented or recessed portion configured to be contacted by a user, whereas the protrusion forming the adjustment mechanism 81 of the first wearable device 10 can be a tab with planar surface that is configured to be contacted by the user.

Further, the carriage 80 of the second wearable device 11 can include an extended portion 39 configured to extend through an elongated aperture 125 formed in the second molded piece 20B of the housing 20. The extended portion 39 can be configured to be inserted in and coupled to the second extending structure 40 e.g., via a friction fit or interference fit of tabs or ribs located on an end or other portion of the extended portion 39 within an opening in the second extending structure 40. In contrast, second molded piece 20B of the housing 20 of the first wearable device 10 does not include an elongated aperture 125. Instead and as mentioned, the carriage 80 of the first wearable device 10 can be located within the channel 85 formed in an exterior bottom surface of in the second molded piece 20B of the housing 20 and the second extending structure 40 can be coupled directly to a substantially planar surface of the carriage 80, as illustrated in FIGS. 2A-2C.

FIGS. 4A-4C are views of a third wearable device 12, while FIGS. 5A-5C are views of a fourth wearable device 13. The third wearable device 12 and the fourth wearable device 13 are analogous the first wearable device 10 with the change that the adjustment mechanism 81 and components associated therewith (e.g., in contact therewith) are different. For instance, FIGS. 4A-4C illustrate embodiments where the adjustment mechanism is manifested as a rotatable lever, while FIGS. 5A-5C illustrate embodiments where the adjustment mechanisms is manifested as a rotatable wheel. Hence, each of FIGS. 5A-5C illustrate embodiments wherein the adjustment mechanism is manifested as a rotatable adjustment mechanism.

The rotatable adjustment mechanisms may be rotatable, at least partially, about an annulus 93 and/or a peg 104 disposed in the annulus 93. The range of rotation may be anywhere from about 10 degrees to about 360 degrees. For instance, the rotatable lever illustrated in FIGS. 4A-4C and the rotatable wheel illustrated in FIG. 5A-5C may have a range of rotation that is in a range from about 10 to about 50 degrees. Each of the rotatable lever and the rotatable wheel may include a portion that is exposed from the housing 20 (e.g., a lever or a portion of the wheel) can is configured to be contacted by a user to impart rotation of the rotatably adjustment mechanism. That is, the housing 20 can include a slot or opening 90 at an end of the housing 20 that permits the wheel or the lever to protrude (e.g., in a substantially longitudinally direction) and extend a distance from the housing 20, while another portion of the adjustment mechanism 81 is disposed in the housing. For instance, the lever or a portion of the wheel can extend extending laterally through the slot 90 to a position outside of the housing 20, thereby permitting a user to actuate (rotate) the rotatable adjustment via contact with the lever or portion of the wheel outside of the housing 20.

As mentioned, the second extending structure 40 can be coupled to the carriage 80. As illustrated in FIGS. 4A-4C and 5A-5C, the carriage 80 of the third and fourth wearable devices 12, 13 can include an extended portion 39 configured to extend through an elongated aperture 125 formed in the second molded piece 20B of the housing 20. The extended portion 39 can be configured to be inserted in or around and otherwise be coupled to the second extending structure 40 e.g., via a friction fit or interference fit of tabs or ribs located on an end or other portion of the extended portion 39 within an opening in the second extending structure 40. These are merely examples, alternate or additional mechanisms of coupling the components herein are possible such as coupling components together via a first connector (e.g., male connector) associated with or integral with a first component and a second corresponding connector (e.g., a female connector) associated with or integral with a second component, via a latch and/or pin in conjunction with a corresponding aperture, etc. are possible.

Continuing with the description of FIGS. 4A-4C and 5A-5C, the portion of the adjustment mechanism 81 that is disposed within the housing can include a notched or tooth structure (e.g., notches 94 as illustrated in FIG. 4C) that is configured to interface with (interference fit with) a corresponding notched or tooth structure (e.g., notches 92 as illustrated in FIG. 4C) on the carriage 80. Hence, rotation of the rotatable lever or rotatable wheel in a given direction (e.g., in the direction 73) and be translated via the interface between the corresponding notched or tooth structures to impart longitudinal movement (e.g., in the direction 75) of the carriage 80. For instance, rotation of the wheel or lever in a first direction can impart movement of the carriage 80 longitudinally toward the first extending structure 30, while rotation of the wheel or lever in a second direction (opposing the first direction) can impart movement of the carriage 80 longitudinally away from the first extending structures 30. Thus, rotation of the lever or wheel permit a user to readily adjust that distance 31 between the first extending structure 30 and the second extending structure 40.

For instance, the lever can rotate from a first position (at a first end of the slot extending through the first end of the housing), as illustrated in FIG. 4A, to a second position at a second end of the slot extending through the first end of the housing 20 that is opposite the first end of the slot), as illustrated in FIG. 4B. Similarly, the wheel can include a curvilinear slot 91 extending within a portion of the wheel. In such instances, a first end or portion of another peg or projection element (not shown) can be disposed within the curvilinear slot 91 and a second end of the peg or projection can be fixable coupled to the housing 20 such that the peg or projection can limit the degree of rotation of the wheel. For instance, the wheel can be configured to move between a first position (where the peg or projection is located at a first end of the curvilinear slot), as illustrated in FIG. 5A, to a second position (where the peg or projection is located at a second end of the curvilinear slot opposite the first end of the curvilinear slot, as illustrated in FIG. 5B.

In some embodiments, a spring or other mechanism can disposition the carriage 80 of any one of the devices 10, 11, 12, or 13 to given position. When present, the dispositioning mechanism can be coupled to the carriage 80 or the adjustment mechanism. For instance, the dispositioning mechanism can be a spring that is directly coupled to the carriage 80 (e.g., having one end coupled to the carriage and another end coupled to the housing), among other possibilities. Similarly, in some embodiments the dispositioning mechanism can be a spring. The disposition mechanism (not shown) can be configured to disposition the carriage 80 to a longitudinally extended position or can be configured to disposition the carriage to a longitudinally contracted position.

In some embodiments, the carriage 80 can include an elongated slot 98 extending longitudinally along a portion of the length of the carriage 80. In such embodiments, a projection or peg can be configured to be slidably disposed within the elongated slot 98. For instance, the lower lid 20B can include an elongated peg 99 or other shaped protrusion configured to extend into the elongated longitudinal slot 98, as illustrated in FIG. 4C. The presence of the elongated slot 98 and the corresponding protrusion or elongated peg 99 can promote aspects herein such as ensuring that the carriage 80 is configured to translate in a substantially longitudinal direction within the housing 20.

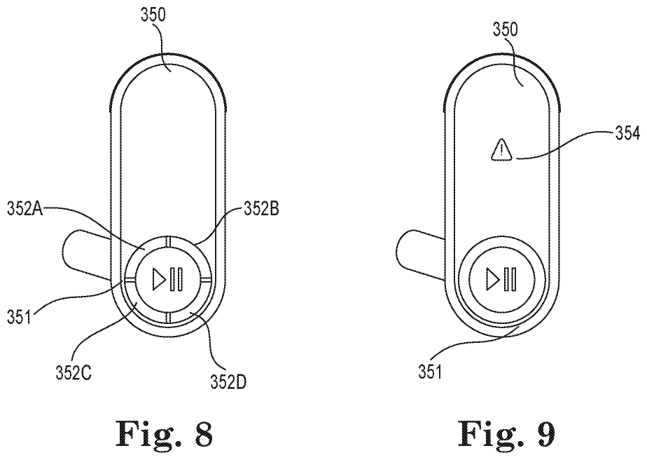

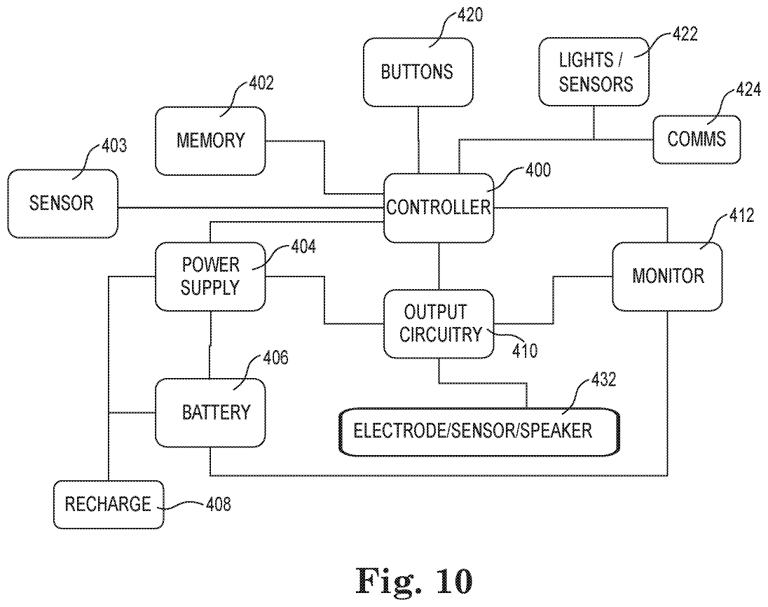

FIG. 6 illustrates placement of a wearable device. The device 250 includes an anchor arm 252. A button 254 is provided for starting or stopping (e.g., pausing) an audio output of the device 250. A tap design, rather than button 254, may be used if desired. Indicator LEDs may be provided at 256 and/or 258. Alternatively, or in addition, the device 250 can include a battery status ring (e.g., the battery status ring 351 as illustrated in FIGS. 8 and 9, herein). A speaker may be used to provide audible indications of status, such as by inclusion on the anchor arm 252 or elsewhere on the device. Alternatively, or in addition to the battery status ring and/or other controls or indicators depicted on the device 250, a separate device such as a smartphone, tablet, or laptop computer, etc. and/or a special purpose programmer or user device may be coupled in a wired or wireless manner to the device 250. This device may be configured with a software and/or firmware to communicate with the device 250. For instance, the device may be configured to control and/or monitor aspects of the device 250 such as starting, stopping, and/or otherwise modifying operation (e.g., audio output) of the device 250.

The device 250, with anchor arm 252, and an element such as a speaker, electrode, vibrating element and/or audio output device as in any of the preceding versions of a wearable device, will be placed as indicated by the arrows. The anchor arm 252 passes behind the tragus, and/or into the auditory canal. This brings the element such as a speaker to the position marked speaker location, and the second extending structure to the position marked second extending structure. Such positioning would also put the element such as the speaker on/at the conchae caverna, and the second extending structure on/at the conchae cymba. In other examples, the element may be differently placed, and/or more than one element can be used. Further, rather than or in addition to a given element such as a speaker, another type of element such as an electrode, a vibrating element, and/or a sensor such as those described herein may be used, as desired, singly or in combinations. Similarly, such positioning would put an element such as the first electrode on/at the conchae caverna, and other element such as the second electrode on/at the conchae cymba, in embodiments with such electrodes. In other examples, one or the other of the electrodes may be differently placed, and/or more than two electrodes can be used. Further, rather than electrodes, other vagus nerve stimulation elements may be used, as desired, singly or in combinations.

As indicated by line/arrow 260, in several examples the device may be positioned by inserting the anchor arm 252 into the auditory canal, and/or beneath the tragus, and then twisting the device. Some examples may twist the device in a superior/anterior direction, bringing the second extending structure (not shown) into a position abutting the anatomy of the exterior of the ear, such as a superior portion at the posterior edge of the crus helix, marked at 262. This positions the housing more vertically in the ear, with the end opposite the anchor arm 252 near the superior helix. Other examples twist in the opposite direction, in an inferior/posterior direction, bringing the second extending structure (again, not shown) into a position abutting the anatomy of the exterior of the ear, such as the antihelix, as indicated at 264. Whether the device is twisted or not, once placed within the ear, expansion of the extending structures creates force between portions of the boundary of the cavum (ear canal, tragus, and antitragus) against portions of the boundary of the cymba (helix, crus of helix, and antihelix) to secure the device within the ear.

This twisting step highlighted at 260 works the device into a desired position, and can be performed by the user in a simple, quick installation step. In some examples, no molding, curing or reshaping is needed. Because the second extending structure has a variable length, such as by including therein a resilient member or spring, such twisting allows the device to more or less automatically achieve a desirable position in which the electrodes, speakers, or sensor(s) are positioned against or proximate to the user's skin. An adhesive strip, such as tape or other substrate material, can be added if needed to maintain device positioning, however it is envisioned that an additional piece of tap will not be needed for most users, again simplifying the use of the system for the user.

Optionally, if a clip is used, the clip would pass over the helix, for example at a superior or posterior location, or elsewhere and/or in-between, to hold the device in place. Optionally, if tape is used, the tape may extend to and over the region marked superior helix, extend to and over the region marked posterior helix, or elsewhere.

Regardless of the optional clip or tape inclusion, the device 250, using the anchor arm 252 and the extending structures described herein, is configured for placement such that the entire device, in some examples, is positioned inside the periphery of the ear, with no wires extending therefrom. In other examples, a wire does extend out to a return electrode positioned elsewhere on the patient, such as the torso or neck, if desired. In some examples, only a single device 250 is present in the system, omitting a second device positioned on the other ear. In some examples, only a single device 250 is present in the system, omitting a second device positioned on the other ear. The device 250 may be configured for positioning on the left ear, as may be inferred from FIG. 6. Alternatively, the device may be configured for positioning on the right ear, if desired.

Some examples may include two devices 250 that are separately positioned, without mechanical/electrical contact therebetween, one for each ear of a user. For such as “two-device” system, audio output can be delivered independently by a respective speaker located in each device, in some examples. In other examples, may be coordinated such as by providing wireless communication circuitry in each device so that the two devices can communicate with one another to coordinate audio delivery, or so that each device can communicate with another device such as a user's smartphone (operating an application specific to the system) that communicates with each device to synchronize or coordinating audio delivery.

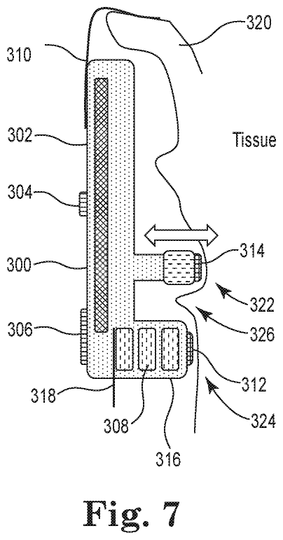

FIG. 7 is a side section views illustrating mechanisms for securing a wearable device to the ear. In FIG. 7, a device 300 is shown secured to the user's ear. Due to the capability of the devices herein to vary a length thereof (e.g., a variable length between the first and second extending members), in some embodiments the wearable devices herein can be secured to the user's ear in the absence of another element (e.g., tape or other type of additional element). However, in some embodiments, tape or another type of adhesive element can optionally be attached to the device 300 and the superior helix 320 to promote retention of the wearable device in the ear. In some embodiments, the device is sized and shaped so that when positioned as shown, the first extending structure 330 is positioned at the conchae caverna 324, and the second extending structure 340 rests against tissue at the conchae cymba 322. The positioning can also be characterized as having the first extending structure 330 and second extending structure 340 positioned on opposing sides of the crus of helix 326. For instance, the positioning can also be characterized as having the first electrode (represented as element 312) and the second electrode (represented as element 314) positioned on opposing sides of the crus of helix 326. As mentioned, the first extending structure 330, the second extending structure 340, or both, can include an audio output device. For instance, an element 314 such as a first electrode and/or a speaker can be located on or near an end of the second extending structure 340, as illustrated in FIG. 7. In some embodiments, the element 314 can be located on or near the end region the second extending structure 340 and another element 312 such as a second electrode and/or a sensor such as those described herein can be on or near the end of the first extending structure 330, as illustrated in FIG. 7. Alternatively or in addition, another audio output device (e.g., a speaker), another electrode, and/or another sensor can be located at a different location in the devices herein. Due to the capability of the devices herein to vary a length thereof, in some embodiments the wearable device can be secured to the ear without any of an additional anchor arm, clip, and adhesive tape. In such embodiments, the devices herein can be characterized in part by an absence of each of anchor arm (e.g., a C-shaped or other shaped protrusion configured to extend around a portion of an ear), clip, and adhesive tape.

Illustratively, and without limitation to a particular layout, the device 300 is shown having a printed circuit board 302 therein, coupled by feedthrough or other wires (not shown) to the alert indicators 304, on/off/pause button 306, first electrode or sensor 312 and a second electrode or an audio output device (e.g., speaker) 314. That is, in some embodiments, element 312 can be a first electrode and element 314 can be a second electrode. However, in some embodiments, element 312 can be a sensor and/or element 314 can be a an audio output device. A stack of battery cells 308, which may be standard button cells or may be a custom design, is contained in this example in the first extending structure 316. Other layouts and battery types can be used; any number of battery cells may be used, though it is expected generally that one to three cells would be used. The device may be a single use device (where single use means use for a single user for a limited period of time e.g., up to one month, or up to fifteen days, for example, and/or where single use indicates the batteries 308 are not replaceable). In other examples, the device may have rechargeable or replaceable batteries 308 and is adapted for repeated use. A removeable tab 318 may be used to preserve battery capacity prior to use; once the tab 318 is removed, the electrical circuit for powering the device is completed and the device electronics are operational. In some examples, an optical light pipe such as an optical fiber may be used to transmit light from LEDs on the circuit board 302 to desired positions, for instance for use on the indicators.

FIGS. 8-9 provides an illustrative example of status and warning lights. The illustrative device 350 can correspond to any of the devices 10, 11, 12, and/or 13 described herein. Alert indicators (lights typically) can include power indicators (e.g., on/off indicators), connectivity (e.g., WI-FI and/or BLUETOOTH) indicators, and/or or a low battery alert 354. Other alerts and mechanisms for interaction with the user may be used. A digital screen can be used if desired instead of discrete alert lights.

The multi-purpose light emitting diode ring 351 can be configured to indicate battery health or status and/or other aspects of device operation by illuminating, flashing, and/or turning off some or all the progress lights segments which comprise the ring. For instance, the multi-purpose light emitting diode ring 351 can be configured to incrementally indicate an incremental reduction in battery charge by flashing or illuminating an individual segment to indicate that corresponding battery charge level. For example, the multi-purpose light emitting diode ring 351 can be configured to incrementally indicate a battery charge level by turning off the individual segment corresponding to a range of battery charge levels (e.g., from 100 percent to 75 percent charged) responsive to the battery charge being reduced to a battery charge level (e.g., 73 percent) that is less than the range of battery charge levels and flashing a subsequent individual segment to indicate that the battery charge level is within a subsequent (lower) battery charge level during a subsequent corresponding segment of time, as detailed herein. However, other mechanisms to indicate battery charge level and/or device status/operation (e.g., changing a color, varying an intensity, etc. of the status lights can be utilized alternatively or additional to indicate device status and/or battery charge level. The multi-purpose light emitting diode ring 351 can be configured in a in a clock-like circular pattern to indicate battery charge level and/or can include device status indicators. For example, the progress ring can be formed of various segments representative of distinct portions or ranges of battery charge levels. For instance, as illustrated in FIGS. 8 and 9 the ring 351 can have four light segments 352A, 352B, 352C, 352D (collectively referred to herein as light segments 352). The light segments 352 can correspond to a range of respective battery charge levels (e.g., each segment corresponds to a about 25 percent of an overall battery charge level). A liquid crystal display, touchscreen, or other display may be used instead of the ring 351, if desired.

As illustrated in FIGS. 8-9, the device 350 can include one or more alert lights that can be selectively displayed or illuminated. Alert lights may include, for example, lights that indicate problems with the device, which may include poor contact with the skin (determined for example using a temperature sensor or an impedance monitor, as desired) when the device includes a sensor such as the sensor 312 in FIG. 7, expiration of the device, other failure in the device, low battery, etc. A button may be used to initiate or pause the audio output, as desired. As illustrated in FIGS. 8-9, the status ring 351, the light segments 352, and/or the alert lights 354 can be located on an exterior surface (e.g., an exterior surface of the first molded piece 20A, as described herein) of the device 350. Having the status ring 351, the light segments 352, and/or the alert lights 354 be located on the exterior surface of the device can promote aspects herein such as permitting access to and/or permitting the status ring 351, the light segments 352, and/or the alert lights 354 to be readily viewed (e.g., be visible) even when the device is positioned in in an ear of a user). However, the quantity, the type, and/or the location of the status ring 351, the light segments 352, and/or the alert lights 354 can be varied.

FIG. 10 is a block diagram for illustrative circuitry. The illustrative circuitry may be described as operational circuitry for the device, and would be contained in the housing as shown in any of the preceding examples. The device includes a controller 400. The controller 400 may take many forms, including, for example, a microcontroller or microprocessor, coupled to a memory 402 storing readable instructions for performing methods as described herein, as well as providing configuration of the controller for the various examples that follow. The controller 400 may include one more application-specific integrated circuits (ASIC) to provide additional or specialized functionality, such as, without limitation, a signal processing ASIC that can filter received signals from a sensor 403 such as those described herein using digital filtering techniques. Logic circuitry, state machines, and discrete or integrated circuit components may be included as well. A controller 400 may take the form of a state machine, if desired. The skilled person will recognize many different hardware implementations are available for a controller. Likewise, the memory 402 can take any suitable form, including Flash memory, combinations of multiple memory types, etc.

The operational circuitry also includes a power supply block 404, coupled to a battery 406. The power supply block may include voltage step-up or step-down circuitry, or may include appropriate regulators, converters and the like, as well as smoothing circuitry as needed/desired to obtain power from a battery 406 and provide power at specified voltage/current for use in the controller 400 as well as the output circuitry shown at 410. One, two, three, four or more battery cells may form a battery 406; commercial off-the shelf button-type batteries may be used, or specialized versions may be developed and used. For example, three or four lithium-chemistry button batteries may provide 9 or 12 volts of power supply, allowing maximum currents in the device to stay relatively small (reducing heat), while generating sufficient headroom to provide desired current or voltage levels for therapy. Batteries may be replaceable, if desired. Rechargeable batteries could be used, whether removeable and rechargeable or by providing a recharging circuit as indicated at 408, in the device, where power can be transferred to a recharging circuit by use of an electrical port on the device, or by wireless transmission (inductive, RF, ultrasonic, etc.) to a transducer on or inside the device. An example may use an inductive loop coupled to a rectification circuit that in turn delivers current/power to the battery 406 for recharging, for example. A recharging case or cord, for example, can be used to permit recharging of the device or devices. Some examples may include electrical contacts on the device for recharging in a recharging case/housing, if desired.

The power supply 404 may further include a dedicated voltage converter to provide, for example, a source for a current controlled output circuitry. In an example, an inductive or capacitive step-up circuit is used to store a 60-volt amplitude on one or more capacitors to provide headroom for a current controller output circuit using, for example, one or more current mirrors to control the output current. Suitable amplifier-based circuits may be used, instead, or any other desired circuit can be used. While inductive step-up circuitry can be used, capacitive converter designs may provide better MRI-compatibility and tend to be smaller and introduce less weight.

The output circuitry 410 may include a set of switches, such as an H-Bridge circuitry design, configured to provide alternating signal outputs. Square wave outputs may be used, and may be current controlled or voltage controlled, as desired. Non-square waves can be used as well, such as exponentially decaying, sinusoidal (in which case a resonant circuit can be included), triangle, ramped, etc. The power supply 404 is configured to provide voltage step-up (such as a voltage multiplier using inducive or capacitive elements), allowing the output circuitry to shape and control the power signals issued to the electrode, sensor, and/or transducer 432 such as those described herein. The electrode, sensor, and/or transducer 432 may also receive control signals from the controller 400 to manage, for example, output frequency of the transducer, depending on design.

The monitoring circuitry 412 can include one or more sensors such as those described herein. For example, the monitoring circuitry can be manifested as a temperature sensor (such as a thermistor, resistance temperature detectors, thermocouples, and/or integrated circuit sensors) to monitor temperature at the tissue interface.

The monitoring circuitry 412 may also monitor battery status, including, for example, a current sensor or coulomb counter if desired to track actual battery use, or a voltage sensor to determine open, lightly loaded, or loaded output voltage of the battery 406 or individual cells therein. Battery usage may instead be tracked, for the purpose of determining battery end of life/status, by the controller 400 using timers, etc., as desired.

The memory 402 may store controller-readable instructions for operating the device in any suitable form, and can also store operating data, including time spent in pause, on/off or other operational data. Operational data may include temperature or impedance data, if desired, or any other sensed parameters or signal.

The controller is also coupled to what may be termed input-output devices, including any buttons 420 on the device, sensors, and/or lights 422 described herein. A screen or touchscreen may be used instead or as well as those items shown. Some systems may optionally include an RF circuit block 424, including, for example and without limitation, Bluetooth, WiFi, and/or any wireless communications circuitry (antenna, driver, crystal/resonator, etc.) for performing wireless communication with a separate device. For example, a smartphone operating an application may communicate via Bluetooth with the device to control any characteristic of device operation (duration, on/off, repetition rate, amplitude, pulse width, type, etc.) and/or to obtain device operational data (usage, battery status, etc.).

General purpose devices may communicate with the system if desired, using for example an application operating on a smartphone, tablet, or computer. Communication may be used to modify settings, upload new software to the device, and/or to download device and/or application usage data or other usage data. Device status, such as battery capacity, may be communicated. Communication may also be used to turn the device on or off, if desired, rather than relying on a button or other actuatable component on the device and/or device housing.

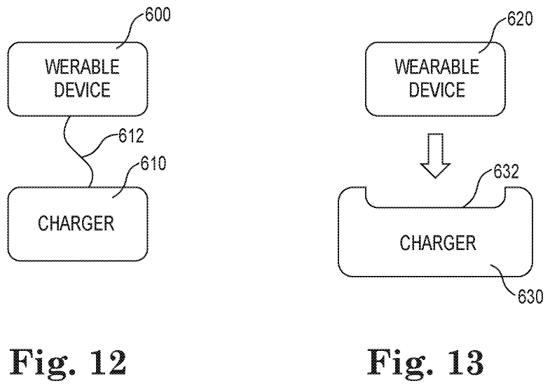

FIG. 11 shows a block process flow diagram of an illustrative method. A relatively comprehensive method is shown; some other or alternative examples may omit one or more blocks/steps, or may replace the illustrative steps shown with other steps. For instance, the methods herein can additionally include adjusting the actuating the adjustment mechanisms to vary an adjustable distance between the first extending structure and the second extending structure until the second extending structure abuts the helix, the antihelix, the inferior crus, or any combination thereof, as described herein. For example, the method can include placing the anchor arm in the auditory canal or at least partially beneath the tragus, as indicated at 504. Further, the methods herein can include adjusting the adjustable distance prior to, during, and/or subsequent to placing the devices (e.g., an anchor arm of the devices) in the auditory canal. For example, the adjustment can be performed subsequently to placing the anchor arm in the auditory canal or at least partially beneath the tragus. As mentioned, the adjustment mechanisms herein can be actuated with one or more digits on an individual hand of a user, as indicated at 506. Hence, the adjustment mechanisms herein can permit adjustment with one hand (an individual) hand of a user. The adjustment can be performed by way of rotation or translation of the adjustment mechanism e.g., relative to the housing 20, unlike some device that require pinching or the use of two hands to adjust aspects of a device.

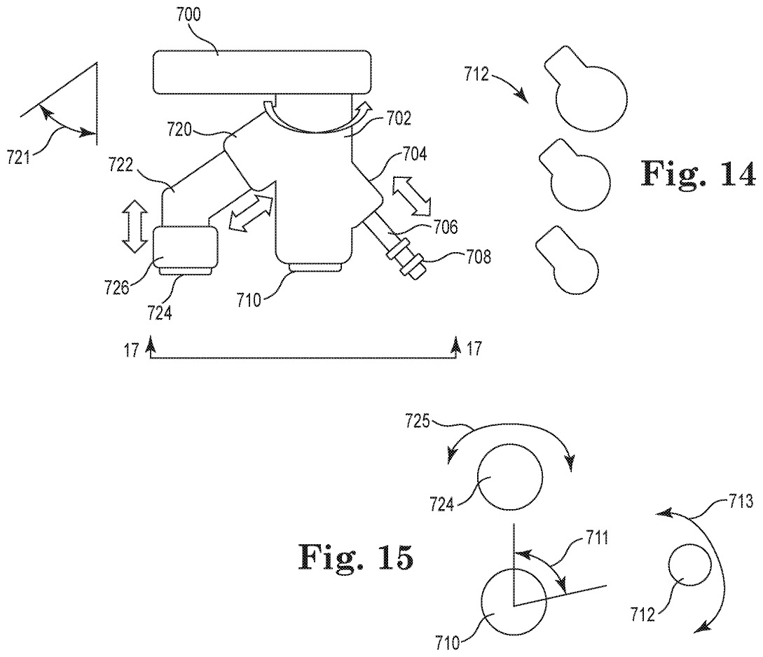

FIGS. 12-13 show wearable devices with chargers. For a user to use a device in a long-term sense, the power supply must be either replaceable (such as with replaceable batteries) or replenishable. A rechargeable wearable device may be useful in any context. In FIG. 12, a wearable device 600 is shown connected to a charger 610 using a wire 612. The connection may use standard connectors, such as uniform serial bus (USB) connectors, micro-USB, etc., or may be a special purpose connector 612 to prevent unauthorized use or modification of the wearable device 600, if desired. The charger 610 may be battery powered or may use wall power, as desired. The wearable device 600 may remain positioned in the ear of a user during charging, or may be removed. Another example is shown at FIG. 13. Here the wearable device 620 is received in a charger 630, having a depression or cradle 632 for receiving the wearable device 620. Electrical connectors can be provided in the cradle 632, positioned to align with electrical connections on the outside of the wearable device 620. Other modes of power transmission can be used, including inductive, RF, optical, etc., as desired.

In each of FIGS. 12-13, data transmission can be performed while charging takes place. For example, firmware in the wearable devices 600, 620 may be updated, or settings modified, as desired. The chargers 610, 630 may be connectable to additional devices, such as a smartphone operating a dedicated application for the purpose of software updating, and/or wearable device operation modification, if desired.

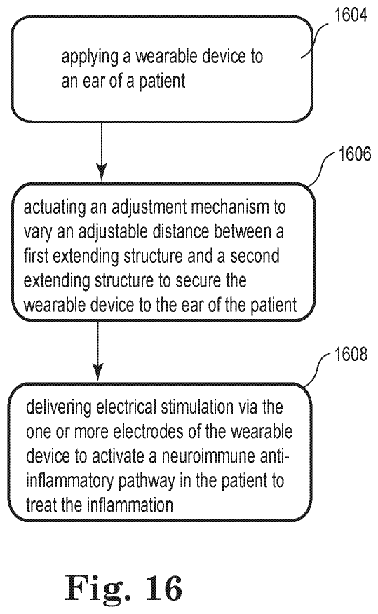

FIGS. 14-15 illustrate further structures. In FIG. 14, a wearable device 700 includes an extending structure 702 which carries an anchor arm 704, having an extending anchor arm 706 moveably mounted therein as indicated by the arrow. Ridges are provided as shown at 708 for holding a removable tip thereon, to anchor in the auditory canal of the user's ear. A speaker is represented by element 710. When the device is placed in an ear, the speaker 710 may be placed in or adjacent to the auditory canal of a user. A plurality of tips 712, in a range of sizes if desired, are provided with the device. Tips 712 may come in various shapes or sizes to allow different users to select a best fit. Tips 712 may be replaceable, as the position in the auditory canal may lead to wax build up, for example, making occasional or periodic replacement useful.

The extending structure 702 also carries an extension 722 which can be extended or retracted relative to a receiver 720, such as by including a spring-loaded structure, as indicated by the arrow. The extension 722 forms an angle 721 relative to the axis of the extending structure 702, the angle being, illustratively, in the range of about 30 to about 60 degrees; in an example, the angle 721 is about 45 degrees. An optional speaker or sensor 724 is positioned on a carrier 726, which may be a generally hollow piece that can slide over the extension 722, as indicated by the arrow. Positioning may again be spring loaded, if desired. This design has a single extending structure 702 relative to the main body of the device 700.

The extending structure 702 may be rotatable (at least partly) if desired, allowing the main body to directed, vertically, horizonal, or at an angle therebetween when placed on the user. For example, if a user is in a recumbent position the rotation of the extending structure may be used to adjust for comfort and secure positioning. In some examples, the receiver 720 is rotatable relative to the anchor arm 704, for example, allowing different angles to be defined therebetween, if desired.

FIG. 15 shows the orientation of speakers or sensors of FIG. 14. It may be noted that FIG. 14 illustrates the location of the anchor arm 706 relative to the extension 722 at an angle so that the extension 722 can be observed in one drawing. FIG. 15 illustrates these angles with a bit more clarity. The speaker 710, along with the optional sensor 724 and the anchor arm at tip 712 form an angle as shown at 711. The positions of speaker 724 and tip 712 are adjustable as illustrated with arcs 713 and 725, so that the angle 711 can be varied in the range of about 60 degrees to about 135 degrees, or more or less. The angle 711 may be, for example, about 90 degrees, if desired. The angle 711 can be adjustable if desired. In some examples, angle 711 is instead a fixed angle.

In some examples, textured, ridged, disk, or bulbous shapes (or combinations thereof) may be used to aid in securing the device in place by including such shapes on the anchor and/or an extending structure. For example, the three elements 710, 712, and 724 shown in FIG. 15 each represent touch points to tissue of a user. Any one, two or all three of these touch points can include a shape (such as bulbous or disk-shape), ridges, texture or roughening that discourages or prevents passage along or past tissue or a tissue ridge or layer, such as the helix, antihelix, helical crus, intertragal notch, tragus, and/or anti-tragus. Such shape, ridges, texture or roughening may be applied on all sides of any of the three touch points, or only along an outer edge or tissue-contacting side thereof, as desired. By outer edge, the intent is to indicate the portion of any anchor or extending structure that would press against tissue to hold the device in a desired position.

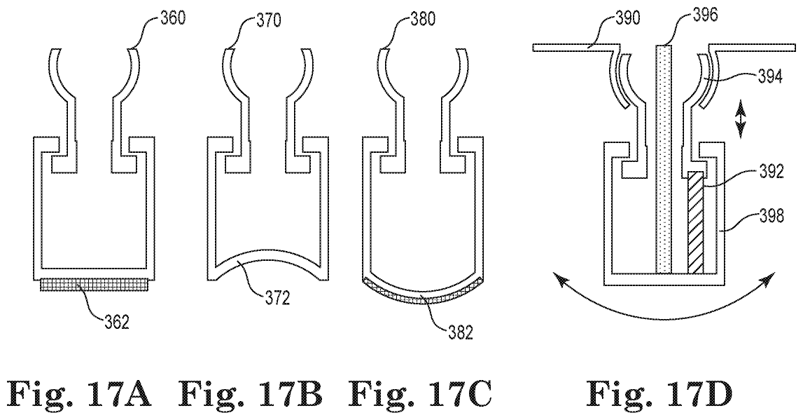

FIG. 16 shows a block process flow diagram of an illustrative method for treating inflammation. The method for treating inflammation can be performed with the devices described herein including those that include at least one electrode, at least one vibrating element (e.g. a piezoelectric actuator), or a combination of one or more electrodes and one or more vibrating elements.

At 1604, the method can include applying a wearable device to an ear of the patient. In some embodiments, applying the wearable device to the ear of the patient can include placing the wearable device in (in direct contact with) the ear of the patient, as detailed herein.

As mentioned, in some embodiments the wearable device can be placed in the ear of the patient without a clip and/or without an adhesive. The wearable device can desirably be adjusted to securely fit within a ear of a user, as detailed herein. For instance, at 1606, the method can include actuating the adjustment mechanism to vary an adjustable distance between the first extending structure and the second extending structure to secure the wearable device to the ear of the patient, as detailed herein.

At 1608, the method can include delivering electrical stimulation via the one or more electrodes of the wearable device. For instance, the electrical stimulation can be delivered to activate a neuroimmune anti-inflammatory pathway in the patient to treat the inflammation. In some embodiments, the stimulation can additionally or alternatively include delivery of oscillations provided via one or more vibrating elements.

In some embodiments, the electrical stimulation can be delivered delivering the electrical stimulation at a current amplitude in a range of about 0.1 mA to about 20 mA, a pulse width in a range of about 10 microseconds to about 20 milliseconds, and/or a frequency in a range of about 1 Hz to about 200 Hz. For instance, the electrical stimulation can be delivered delivering the electrical stimulation at a current amplitude in a range of about 0.1 mA to about 20 mA, a pulse width in a range of about 10 microseconds to about 20 milliseconds, and a frequency in a range of about 1 Hz to about 200 Hz. In some embodiments, the electrical stimulation delivered can be tailored to treat one or more particular types of inflammation. Examples of types of inflammation include inflammation that is associated with one or more conditions selected from the group consisting of: cerebrovascular disorders including stroke and post-stroke inflammatory response; metabolic disorders including diabetes mellitus and glycemic control disorders; drug-induced inflammatory responses including diabetes drug therapy complications; rheumatoid arthritis; inflammatory bowel disease; Alzheimer's disease, Crohn's disease; ulcerative colitis; multiple sclerosis; psoriatic arthritis; osteoarthritis; psoriasis; chronic inflammatory symptoms; and chronic fatigue syndrome.

The device and system may be configured for a variety of use cases. For example, the wearable vagus nerve stimulation can be used in conjunction with pharmacological interventions to treat sepsis in ICU patients. By targeting inflammation including use of vagus nerve stimulation, the system can help modulate the immune response and potentially improve outcomes in patients with severe sepsis.

In another example, in patients with acute respiratory distress syndrome (ARDS) in the hospital setting, wearable vagus nerve stimulation can be utilized alongside mechanical ventilation and anti-inflammatory medications to reduce lung inflammation and improve oxygenation. This combined approach may enhance the overall management of ARDS and potentially speed up the recovery process.

In another example, for patients with severe pneumonia requiring intensive care, wearable vagus nerve stimulation can complement antibiotic therapy and respiratory support by targeting systemic inflammation. By regulating the inflammatory response, this adjunctive therapy may help in reducing the severity of pneumonia and preventing complications in critically ill patients.

In another example, in the management of inflammatory bowel disease (IBD) exacerbations in hospitalized patients, wearable vagus nerve stimulation can be used along with corticosteroids and immunosuppressants to control intestinal inflammation. This combined treatment approach may offer a novel strategy to alleviate symptoms and promote mucosal healing in patients with severe IBD flares.

In another example, wearable vagus nerve stimulation can be combined with pain management techniques in post-operative ICU patients to mitigate surgical inflammation and improve recovery outcomes. By targeting the inflammatory cascade, this adjunct therapy may aid in reducing post-operative complications and enhancing the overall healing process in critically ill surgical patients. In addition, again for the post-surgery context, wearable vagus nerve stimulation can be utilized post-operatively to enhance bowel motility by delivering targeted electrical impulses to the vagus nerve, promoting gastrointestinal motility and reducing the risk of post-operative ileus.

In another example, a wearable vagus nerve stimulation device can be used in conjunction with remote monitoring systems to continuously track the patient's heart rate, blood pressure, and other vital signs. By integrating real-time data from the device with the digital monitoring platform, healthcare providers can quickly identify any signs of worsening heart failure and intervene promptly to prevent readmission.

In an example, wearable vagus nerve stimulation can be used in combination with traditional pharmacological treatments for Congestive Heart Failure (CHF) to reduce readmission rates. By incorporating vagus nerve stimulation into the patient's treatment plan, the device can potentially improve heart function, reduce inflammation, and enhance autonomic balance, leading to better overall outcomes and decreased risk of hospital readmission.

In another example, wearable vagus nerve stimulation can be used exclusively for mitigation of pain related to medical intervention or medical conditions. Such medical interventions may include, but are not limited to, surgical procedures such as orthopedic surgeries (joint replacements, arthroscopic procedures, spinal fusion), cardiac interventions (bypass surgery, valve replacement, catheter-based procedures), abdominal surgeries (laparoscopic procedures, appendectomy, hernia repair), dental and oral surgeries, cosmetic and reconstructive procedures, neurosurgical interventions, and minimally invasive procedures including endoscopies, colonoscopies, and bronchoscopies. Additionally, the system may be employed during medical treatments such as chemotherapy administration, radiation therapy, injection-based therapies, tissue biopsies, wound debridement, and physical rehabilitation procedures.

The wearable vagus nerve stimulation device may also provide therapeutic benefit for pain associated with medical conditions characterized by inflammatory processes, including but not limited to rheumatoid arthritis, osteoarthritis, inflammatory bowel disease (Crohn's disease, ulcerative colitis), fibromyalgia, neuropathic pain syndromes, autoimmune disorders (lupus, multiple sclerosis), chronic regional pain syndrome, temporomandibular joint disorders, chronic headache and migraine conditions, post-surgical chronic pain, cancer-related pain, and chronic low back pain, where the anti-inflammatory effects of vagus nerve stimulation may reduce both local and systemic inflammatory mediators contributing to pain perception and tissue sensitization.

In another example, wearable vagus nerve stimulation can be used exclusively for mitigation of stress-related hyperglycemia associated with medical intervention or medical conditions. Such medical interventions may include, but are not limited to, surgical procedures such as cardiac surgery (coronary artery bypass, valve replacement), major abdominal surgeries (liver resection, pancreaticoduodenectomy, bowel resection), orthopedic procedures (joint replacement, spinal surgery), neurosurgical interventions (craniotomy, tumor resection), trauma surgery, organ transplantation procedures, and emergency surgical interventions. Additionally, the system may be employed during acute medical treatments such as intensive care unit admissions, mechanical ventilation, hemodialysis, chemotherapy infusion, radiation therapy, invasive diagnostic procedures (cardiac catheterization, bronchoscopy), burn treatment, and critical illness management where physiological stress responses elevate blood glucose levels. The wearable vagus nerve stimulation device may also provide therapeutic benefit for stress-related hyperglycemia associated with medical conditions including diabetes mellitus (both Type 1 and Type 2), prediabetic conditions, metabolic syndrome, acute myocardial infarction, stroke, sepsis, acute pancreatitis, chronic kidney disease, liver cirrhosis, chronic obstructive pulmonary disease exacerbations, psychiatric disorders with associated metabolic dysfunction (depression, anxiety disorders, post-traumatic stress disorder), chronic pain syndromes, sleep disorders, and endocrine disorders (Cushing's syndrome, hyperthyroidism), where vagus nerve stimulation may enhance parasympathetic tone, improve insulin sensitivity, reduce cortisol release, and modulate the stress-induced activation of the hypothalamic-pituitary-adrenal axis that contributes to elevated blood glucose levels.

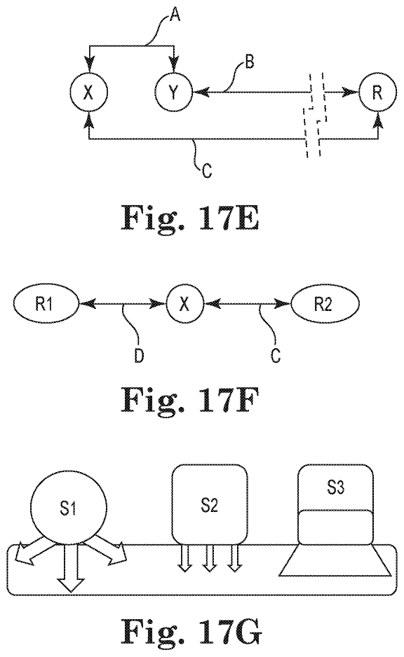

FIGS. 17A-17G show illustrative stimulation device designs. FIG. 17A shows an electrode assembly at 360, having a flat contact surface on which a hydrogel or other tissue coupling element 362 is provided. An adhesive, for example, may attach the coupling element 362 to the electrode assembly 360. FIG. 17B shows an alternative 370 with a concave contact region 372, which may help hold/contain the tissue coupling element and/or a hydrogel or other gel for aiding in signal transmission across the tissue-stimulation element interface. In some instances, an electrode may have a surface that is textured, roughened, scored, or dimpled to increase effective surface area, to thereby lower impedance, in addition serving to receive or retain coupling material, gel, and/or adhesive. Not only electrodes, but any of the described vagus nerve stimulating elements may have textured, roughened, scored or dimpled surfaces, or may comprise an adhesive layer, or may receive a piece of coupling material, gel or adhesive thereon. FIG. 17C shows an alternative 380 having a convex contact surface 382.

FIG. 17D highlights two different ways that the electrode 398 contact can be made adjustable. A spring or other resilient member 392 or 396 can be used to maintain pressure against the tissue once the device 390 is placed. The resilient member 392 or 396 may be, for example, a coiled spring, a compressible foam, or any other suitable structure able to be compressed and expand after removal of applied force. A swivel or ball-joint structure shown at 394 can allow the electrode to be laterally angled as desired. The resilient member shown at 392 presses against the end of the swivel or ball-joint structure 394; alternatively, the resilient member 396 may be used instead and is shown extending up to the device enclosure, for example, all the way to the circuit board, on which an electrical connection can be made so that resilient member 396 serves also as the electrical contact to the electrode 398.

FIG. 17E illustrates different approaches to delivery of electrical stimulation. An electrical output requires at least two “poles” for delivery. A bipolar delivery occurs between two relatively closely spaced electrodes. For example, some of the designs shown above include first and second electrodes disposed, respectively, on the conchae caverna and conchae cymba; when electrical signal passes between two such electrodes, a bipolar output is generated. Line A, between electrodes X and Y in FIG. 17E can be understood as indicating a bipolar output. On the other hand, a monopolar delivery occurs between a first electrode positioned at a therapy site, and a remote electrode located away from the therapy target. Lines B (between electrode Y and a remote electrode, R) and C (between electrode X and a remote electrode, R) indicate monopolar therapy combinations. Multiple return electrodes can be used, for example to influence voltage fields and spread, focus or steer the outputs, such as shown in FIG. 17F where electrode X issues a therapy output with two return electrodes. The return electrode in FIGS. 17E and 17F may be positioned at any desired distance, as indicated with the use of the broken line gaps. Each of these different types of therapy may be used in various examples. Some examples will omit the remote electrode, R.

Other therapy modalities may not require paired neural stimulation elements. For example, as indicated in FIG. 17G, some stimulation elements, such as S1, can generate output stimulation that travels in a range of directions. Vibration/acoustic stimulation via a vibration element such as a vibration element located on the first anchor arm, the second anchor arm, or both, as well as magnetic stimulation, may travel in this way, such that S1 may be understood as an electro-mechanical, or electro-magnetic transducer. Thermal therapy can be generated as well, and so S1 may instead be a thermal element such as a resistor that converts electrical current to heat. Some optical outputs provide collimated light outputs, such as light emitting diodes and/or vertical cavity surface emitting lasers, as illustrated at S2. A dispersing lens may be included as shown for S3, to provide a spread the output light energy; alternatively, a less directional light source can be used, as desired.

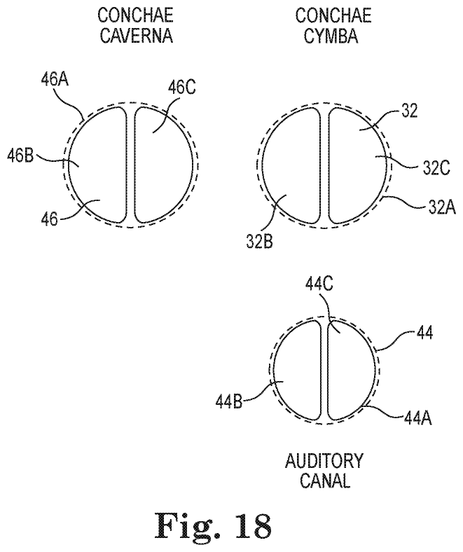

FIG. 18 illustrates various electrode configurations that can be used, as desired. Electrode structures are shown at 32, 44, and 46, corresponding to the electrodes that can be used in systems and devices herein. Various examples of system electrode configurations may be achieved. Electrode structure 44 would be positioned on the anchor arm, and is omitted in some examples. The electrode structure 32 may be a single electrode 32a, or may include more than one electrode, such as electrodes 32b, 32c (more than two may be used, if desired). Likewise, the electrode structure 46 may be a single electrode 46a, or may include more than one electrode, such as electrodes 46b, 46c (more than two may be used, if desired). When present, the electrode structure 44 may be a single electrode 44a, or may include more than one electrode, such as electrodes 44b, 44c (more than two may be used, if desired). Various combinations are contemplated:

Electrode structure 32 may include two electrodes 32b, 32c, with therapy delivered between those two electrodes 32b, 32c only, directing therapy to the conchae cymba. Other electrodes may be omitted, or may be present but inactive, or may delivered a separate waveform. Electrode structure 46 may include two electrodes 46b, 46c, with therapy delivered between those two electrodes 46b, 46c only, directing therapy to the conchae caverna. Other electrodes may be omitted, or may be present but inactive, or may delivered a separate waveform.

Each of electrodes 32b, 32c, 46b, 46c may be included in some examples, and therapy may be delivered in sequential anode/cathode pairs, for example as shown here:

| Anode | Cathode |
|---|---|
| 32b | 46b |
| 32c | 46b |
| 32b | 46c |
| 32c | 46c |

Therapy may start at the top row and proceed to each successive row. After one round of such therapy, the sequence may be repeated with opposite polarity, for example. Other combinations and/or sequences can be used. As the skilled person will understand, this approach may require multiple sources in the electronics of the device, providing multiple, independent signals to control individual electrodes. Some other examples may have one electrical source and a plurality of switches to direct therapy signals as desired. Some examples may have multiple electrical signal sources and a set of switches arranged to multi-plex the output signals as desired.

With a larger number of electrodes, additional flexibility is facilitated allowing the electrical field applied to the underlying tissue to be shaped or tailored as desired. Groupings of electrodes may be electrically connected to form larger or smaller effective stimulation areas. Individual or grouped electrodes may be independently controlled to provide varying levels of stimulation so as to shape activation fields to location or depth to preferentially activate underlying tissue, or to avoid or suppress activation of underlying tissue. In some examples, stimulation intensity can be adjusted to account for electrode position/proximity and/or side. For example, larger currents can be delivered with ganged-together electrodes with less concern regarding patient comfort. Also, varying frequencies of stimulation between electrodes may be used to activate, inhibit, or avoid stimulation of underlying tissue by creating interacting activation fields, like beat frequencies, or inferential therapy.

The circuitry in the stimulation device may include multiple outputs that allow for independent control over each electrode and/or plural electrode pairs, if desired, to allow multiple waveforms to be delivered at the same time. For example, a sinusoidal first stimulation signal issued between electrode 32b and electrode 46b at 40 Hz could be output at the same time as a second stimulation signal generated at 30 Hz using electrode 32c and electrode 46c, resulting in a 10 Hz beat frequency arising within the patient tissue. Other “beat” related approaches or interferential signals may be used instead or in addition to these examples.

In some examples, electrodes may be positioned within the external auditory canal to access the auricular branch of the vagus nerve with reduced surface impedance relative to dry auricular skin, where the canal's cartilaginous walls provide close proximity to Arnold's nerve. In other examples, one or more percutaneous electrodes may be introduced through the skin of the auricle to a depth sufficient to reduce impedance and increase selectivity for activation of auricular branch of vagus nerve fibers relative to surface electrode delivery. Such percutaneous electrodes may be microneedle-type or acupuncture-style needle electrodes positioned in the cymba conchae or cavum conchae targeting the known distribution of Arnold's nerve in the auricular conchal region.

Beyond auricular delivery, taVNS may be combined with at least one additional vagal or neuromodulatory modality. Transcutaneous cervical vagal nerve stimulation may be delivered via surface electrodes positioned over the anterior border of the sternocleidomastoid muscle at the level of the carotid bifurcation, targeting the cervical vagus nerve transcutaneously. Intranasal vagal nerve stimulation may be delivered via electrodes positioned within the nasal cavity to access trigeminal-vagal convergent pathways at the brainstem level. Transcranial direct current stimulation or transcranial magnetic stimulation may be combined with taVNS to modulate insular, prefrontal, or brainstem autonomic nuclei. Pulsed electromagnetic field stimulation delivered via a micro-coil positioned over a cervical vagus nerve location provides a contactless approach to cervical vagal activation. Any of these additional modalities may be delivered simultaneously with taVNS or in a time-staggered sequence based on the mechanistic objective of each modality.

### Peri-Intervention Inflammatory Modulation:

The following definitions apply to terms used throughout this disclosure, particularly in the context of peri-intervention inflammatory modulation:

As used herein, “transcutaneous auricular vagal nerve stimulation” or “taVNS” generally refers to non-invasive electrical stimulation of the auricular branch of the vagus nerve (ABVN) delivered through electrodes positioned on the skin of the auricle (external ear). As used herein, “transcutaneous auricular vagal nerve stimulation” or “taVNS” also encompasses delivery of stimulation energy via other stimulation elements as defined herein, including vibratory, optical, ultrasound, magnetic, and thermal modalities. The stimulation is transcutaneous, meaning it is delivered through intact skin without requiring surgical implantation or invasive access. The auricular branch of the vagus nerve innervates specific regions of the external ear, particularly the concha (including the conchae cymba and conchae cavum), tragus, and portions of the auditory canal. Electrical stimulation of these auricular regions activates the ABVN, which projects to the nucleus tractus solitarius in the brainstem and subsequently modulates central autonomic networks and efferent vagal pathways that regulate inflammation, autonomic function, and other physiologic processes.

As used herein, “sub-perceptive” refers to stimulation delivered at an amplitude below the patient's sensory threshold such that the patient does not consciously perceive the stimulation during delivery. Sub-perceptive amplitudes typically range from about 0.05 mA to about 0.5 mA, though the specific threshold varies between individuals based on factors including skin thickness, electrode contact quality, tissue hydration, and individual sensory sensitivity. Sub-perceptive stimulation may preferentially activate central pathways and central network suppression mechanisms with minimal or no peripheral sensation, making it particularly useful for continuous or extended-duration protocols where patient comfort is paramount.

As used herein, the term “peri-intervention period” refers to the time period encompassing before, during, and after a medical intervention, and includes: (i) the pre-intervention phase, which may range from minutes to days before the intervention and includes preconditioning treatments; (ii) the intra-intervention phase, during which the medical intervention is actively being performed; and (iii) the post-intervention phase, which may range from minutes to weeks after completion of the intervention and includes the acute inflammatory response period, complication management period, and recovery period.

As used herein, “preconditioning treatment” refers to taVNS delivered before a medical intervention with the objective of preparing or “priming” the patient's neuroimmune anti-inflammatory pathways to mount a more controlled inflammatory response when the intervention occurs. Preconditioning may involve one or more stimulation sessions delivered hours to days before the intervention, with parameters selected to induce anti-inflammatory cytokine production (particularly IL-10), promote M2 macrophage polarization, enhance autonomic reserve and heart rate variability, reduce baseline inflammatory tone in patients with elevated inflammatory markers, and potentially induce adaptive cellular responses such as heat shock protein expression or antioxidant enzyme upregulation that provide cytoprotection.

As used herein, “burst mode” stimulation comprises delivery of stimulation in discrete bursts or clusters of pulses separated by rest periods. For example, burst mode may deliver a train of high-frequency pulses (such as 5-10 pulses at 50-100 Hz) followed by a rest period (such as 0.5-5 seconds), with this pattern repeated throughout the treatment session. Burst mode stimulation may be particularly useful during critical intervention phases such as reperfusion, where concentrated stimulation delivery may provide maximal anti-inflammatory pathway activation during brief high-risk periods. Burst mode differs from continuous stimulation (100% duty cycle) and from regular intermittent stimulation in that the active stimulation periods deliver clusters of closely-spaced pulses rather than evenly-distributed pulses.

As used herein, stimulation “synchronized with the reperfusion phase” means that taVNS is initiated, intensified, or delivered in a coordinated temporal relationship with restoration of blood flow to previously ischemic tissue. Synchronization may involve initiating stimulation immediately before reperfusion (such as 1-5 minutes before anticipated reperfusion), at the moment of reperfusion (such as concurrent with aortic de-clamping, catheter-based vessel opening, or thrombectomy), or immediately after reperfusion begins (such as within 1-10 minutes of flow restoration). The timing may be coordinated based on the surgical or procedural plan, with the objective of providing maximal anti-inflammatory pathway activation during the critical period when ischemia-reperfusion injury mechanisms are most active, including reactive oxygen species generation, complement activation, and neutrophil infiltration.

As used herein, “adaptively modulated” refers to automatic or semi-automatic adjustment of stimulation parameters in response to monitored physiologic feedback, allowing the therapy to dynamically respond to the patient's changing inflammatory state and physiologic status. Adaptive modulation may be implemented through closed-loop systems that continuously or frequently monitor biomarkers such as heart rate variability, blood pressure variability, body temperature, or measured cytokine levels and automatically adjust parameters including current amplitude, pulse width, frequency, or duty cycle based on predefined algorithms, decision rules, or machine learning models. Adaptive modulation distinguishes from fixed-parameter protocols by providing individualized, real-time optimization of therapy based on the patient's actual response rather than applying uniform predetermined settings.

As used herein, “real-time physiologic feedback” refers to physiologic parameters that are monitored continuously or at frequent intervals (such as every few seconds to every few minutes) during taVNS delivery, allowing for timely parameter adjustments based on the patient's current state. Real-time feedback may include continuously monitored parameters such as heart rate, heart rate variability, blood pressure, blood pressure variability, body temperature, oxygen saturation, and respiratory rate, as well as frequently measured parameters such as inflammatory biomarkers (IL-6, CRP, TNF-α) measured at intervals of minutes to hours using point-of-care or rapid assay technologies. Real-time feedback permits closed-loop adaptive control systems to respond promptly to changes in the patient's condition.

As used herein, “phase-specific inflammatory modulation objectives” refer to the distinct therapeutic goals for each phase of peri-intervention treatment, recognizing that different anti-inflammatory mechanisms and intervention priorities are relevant at different times. Pre-intervention phase objectives include priming anti-inflammatory pathways, inducing protective cytokines, optimizing autonomic reserve, and reducing baseline inflammatory tone. Intra-intervention phase objectives include limiting real-time inflammatory mediator release as tissue trauma or immune activation occurs, maintaining hemodynamic and autonomic stability, and protecting against acute injury mechanisms such as ischemia-reperfusion injury. Post-intervention phase objectives include suppressing ongoing inflammation, enhancing anti-inflammatory and pro-resolution mediators, accelerating the transition to tissue repair, preventing delayed or secondary complications, and supporting functional recovery.

As used herein, “tier-specific protocol” refers to taVNS treatment protocols that are selected and tailored based on the patient's assigned risk stratification tier. Low-risk tier protocols typically employ low intensity parameters (current amplitude 0.05-1.0 mA, pulse width 50-250 s, frequency 1-25 Hz, duty cycle 5-30%), shorter or no preconditioning (single session or omitted), and brief post-intervention treatment (24-72 hours). Moderate-risk tier protocols employ moderate intensity parameters (current amplitude 0.5-4.0 mA, pulse width 100-500 s, frequency 10-50 Hz, duty cycle 25-50%), intermediate preconditioning duration (1-3 days with daily or twice-daily sessions), and post-intervention treatment for 3-7 days. High-risk tier protocols employ high intensity parameters (current amplitude 1.0-10 mA, pulse width 200-2000 s, frequency 25-100 Hz, duty cycle 40-75%), extended preconditioning (3-7 days with twice-daily or more frequent sessions), and prolonged post-intervention treatment (7-14+ days), often with continuous or near-continuous application during acute phases.

As used herein, “risk stratification tier” refers to a classification of patients into categories based on their likelihood of developing inflammatory complications, considering baseline inflammatory biomarkers (such as CRP, IL-6, TNF-α, ferritin), patient medical history (including age, comorbidities, nutritional status, prior inflammatory complications), autonomic function (such as heart rate variability metrics), and intervention type (including complexity, duration, degree of tissue trauma, and expected inflammatory burden). Common risk tiers include low risk (patients with normal inflammatory markers, good autonomic function, minimal comorbidities, undergoing low-complexity interventions), moderate risk (patients with mildly elevated inflammatory markers, modestly reduced autonomic function, some comorbidities, and/or undergoing moderate-complexity interventions), and high risk (patients with significantly elevated inflammatory markers, poor autonomic function, multiple or severe comorbidities, and/or undergoing high-risk interventions).

As used herein, “stimulation elements” refers to one or more transducers, electrodes, or other components positioned on or proximate to auricular tissue that are configured to deliver stimulation energy to activate the auricular branch of the vagus nerve. Stimulation elements may include, without limitation: electrodes configured to deliver electrical stimulation through direct tissue contact; vibration transducers or piezoelectric elements configured to deliver mechanical or acoustic stimulation; optical transducers including light-emitting diodes and laser sources configured to deliver optical stimulation in the infrared, near-infrared, or visible spectrum; ultrasound transducers configured to deliver focused or unfocused acoustic energy; magnetic transducers including inductive coils configured to deliver electromagnetic stimulation; and thermal elements configured to deliver thermal stimulation. A single device may incorporate one or more stimulation elements of the same or different types, and stimulation may be delivered by a single modality or a combination of modalities.

As used herein, “stimulation parameter” refers to any adjustable characteristic of the stimulation delivered by a stimulation element. For electrical stimulation delivered via electrodes, stimulation parameters include current amplitude, voltage amplitude, pulse width, frequency, duty cycle, waveform shape, and inter-pulse interval. For vibratory or acoustic stimulation, stimulation parameters include vibration frequency, vibration amplitude, and duty cycle. For optical stimulation, stimulation parameters include optical intensity, wavelength, pulse duration, and pulse repetition rate. For ultrasound stimulation, stimulation parameters include acoustic intensity, frequency, pulse duration, and duty cycle. For thermal stimulation, stimulation parameters include target temperature, ramp rate, and duration. One or more stimulation parameters may be adjusted to optimize therapeutic effect, and references herein to adjusting stimulation parameters encompass adjustment of parameters appropriate to the stimulation modality in use.

The following sections describe novel applications of transcutaneous auricular vagal nerve stimulation (taVNS) for mitigating inflammatory complications associated with medical interventions. These approaches build upon the device structures and operational principles described above, applying them in new clinical contexts with protocol-driven, phase-specific stimulation strategies.

Medical interventions that trigger inflammatory responses can be broadly categorized based on their primary inflammatory mechanisms. This classification framework provides a rational basis for selecting appropriate taVNS protocols, timing strategies, and parameter ranges to address the specific inflammatory pathways activated by different intervention types.

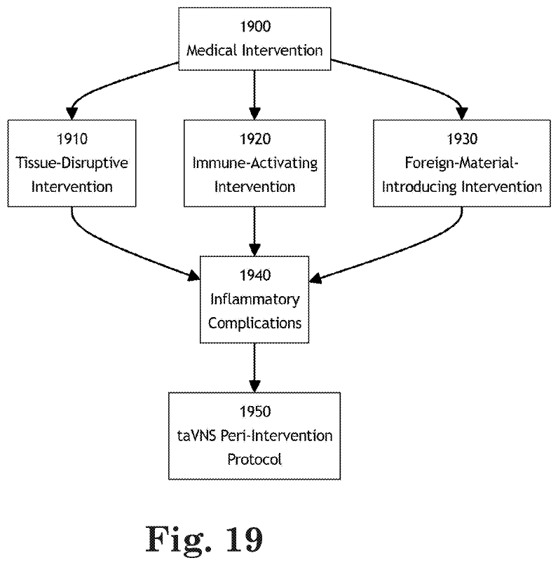

FIG. 19 illustrates the three-category framework for classifying medical interventions based on their inflammatory mechanisms. Medical intervention 1900 represents the universe of interventions that may trigger inflammatory complications. Tissue-disruptive intervention 1910, immune-activating intervention 1920, and foreign-material-introducing intervention 1930 represent the three primary meta-categories. These interventions lead to inflammatory complications 1940, which are addressed by taVNS peri-intervention protocols 1950. While specific interventions may have characteristics of multiple categories (such as organ transplantation spanning all three), identifying the predominant mechanism allows for protocol optimization with modification based on patient-specific factors

### Tissue-Disruptive Interventions (Meta-Category I):

Tissue-disruptive interventions (1910) involve direct physical, thermal, chemical, or radiological disruption of tissue integrity, triggering damage-associated molecular patterns (DAMPs), complement activation, and innate immune cell infiltration. The inflammatory response is characterized by local tissue injury, systemic inflammatory mediator release, and potential progression to systemic inflammatory response syndrome (SIRS).

### Tissue-Disruptive Interventions Encompass Multiple Subcategories:

Surgical Interventions (Subcategory A): This subcategory includes invasive surgical procedures that mechanically disrupt tissue planes, sever blood vessels, and trigger surgical stress responses. Examples include cranial surgery (including craniotomy, craniectomy, tumor resection, aneurysm clipping), spinal surgery (including laminectomy, fusion, discectomy, decompression), cardiothoracic surgery (including coronary artery bypass grafting, valve replacement, thoracotomy, pneumonectomy), abdominal surgery (including laparotomy, bowel resection, liver resection, pancreatectomy), orthopedic surgery (including joint replacement, fracture fixation, spinal instrumentation), vascular surgery (including aortic repair, bypass grafting, endarterectomy), and organ transplantation surgery (which also involves immune activation). The degree of inflammatory response generally correlates with the extent of tissue disruption, duration of surgery, and involvement of highly vascularized organs.

Cardiopulmonary Bypass and Extracorporeal Circulation (Subcategory B): Cardiopulmonary bypass during cardiac surgery triggers particularly intense inflammatory responses through multiple mechanisms including contact activation of blood with artificial surfaces, shear stress, ischemia-reperfusion injury, endotoxin release, and complement activation. The systemic inflammatory response to cardiopulmonary bypass can lead to capillary leak syndrome, acute kidney injury, cerebral dysfunction, and multi-organ complications. Similar inflammatory cascades occur with other forms of extracorporeal circulation including extracorporeal membrane oxygenation (ECMO) and continuous renal replacement therapy, though the inflammatory burden may differ based on circuit design and duration of support.

Radiation Therapy (Subcategory C): Ionizing radiation causes direct DNA damage and triggers inflammatory responses in both target tissues and surrounding normal tissues. Radiation induces expression of inflammatory cytokines, adhesion molecules, and reactive oxygen species. Acute radiation inflammation manifests as mucositis, dermatitis, pneumonitis, or enteritis depending on the treatment field. Chronic radiation inflammation can lead to fibrosis and ongoing tissue damage. The inflammatory response to radiation is influenced by dose, fractionation schedule, treatment volume, and tissue radiosensitivity.

Ablative Therapies (Subcategory D): Radiofrequency ablation, microwave ablation, cryoablation, and high-intensity focused ultrasound (HIFU) create zones of tissue necrosis that trigger inflammatory responses. The necrotic tissue releases DAMPs, activating innate immune responses. The inflammatory reaction contributes to the therapeutic effect by recruiting immune cells that may recognize tumor antigens (in cancer treatment), but can also cause complications including abscess formation, organ dysfunction, or systemic inflammatory responses if large tissue volumes are ablated.

Ischemia-Reperfusion Procedures (Subcategory E): Procedures involving temporary cessation of blood flow followed by restoration of circulation trigger ischemia-reperfusion injury, a particularly intense inflammatory process. Examples include percutaneous coronary intervention for myocardial infarction, thrombectomy for stroke, revascularization procedures for peripheral arterial disease, and organ transplantation. During ischemia, cellular ATP depletion and metabolic derangements prime inflammatory responses. Upon reperfusion, oxygen reintroduction generates reactive oxygen species, complement activation occurs, neutrophils infiltrate tissue, and cytokine cascades are unleashed. The inflammatory injury during reperfusion can paradoxically worsen tissue damage despite restoration of blood flow. Timing of taVNS to coincide with or slightly precede reperfusion may be particularly beneficial in attenuating this injury pattern.

Trauma and Burns (Subcategory F): Severe traumatic injury and burn injury trigger profound inflammatory responses proportional to injury severity. Trauma releases DAMPs from damaged cells, disrupts tissue barriers, may introduce foreign material or bacteria, and activates stress hormone responses. The inflammatory response to major trauma or burns can progress to SIRS, acute respiratory distress syndrome (ARDS), and multi-organ dysfunction syndrome (MODS). While taVNS application in emergency trauma settings may face practical challenges, it could be applied in the early post-injury period to modulate ongoing inflammation.

### Immune-Activating Interventions (Meta-Category II)

Immune-activating interventions (1920) deliberately stimulate, modulate, or supplement the immune system as part of their therapeutic mechanism, but can trigger exaggerated or dysregulated inflammatory responses. These interventions are increasingly important in modern medicine but carry risks of severe inflammatory complications.

### Immune-Activating Interventions Include Multiple Subcategories:

CAR-T Cell Therapy (Subcategory A): Chimeric antigen receptor T-cell therapy involves genetic modification of a patient's T cells to recognize and attack cancer cells. After infusion of CAR-T cells, their expansion and activation against target cells can trigger cytokine release syndrome (CRS), characterized by massive elevation of inflammatory cytokines including IL-6, IL-10, IFN-γ, and others. CRS severity ranges from mild flu-like symptoms to life-threatening shock, capillary leak, and multi-organ failure. Immune effector cell-associated neurotoxicity syndrome (ICANS) represents another serious inflammatory complication involving cerebral edema, encephalopathy, and seizures. Current management of severe CRS includes IL-6 receptor blockade with tocilizumab and/or corticosteroids, but these interventions may compromise the therapeutic efficacy of CAR-T cells. taVNS offers a non-pharmacologic approach to modulate inflammation without directly blocking the intended anti-tumor effects.

Immune Checkpoint Inhibitor Therapy (Subcategory B): Antibodies targeting CTLA-4, PD-1, or PD-L1 unleash anti-tumor immunity by removing inhibitory signals on T cells. However, this immune activation can also trigger immune-related adverse events affecting multiple organ systems. Checkpoint inhibitor-induced inflammation can manifest as colitis, hepatitis, pneumonitis, endocrinopathies, nephritis, myocarditis, or neurologic complications. These immune-related adverse events are managed with immunosuppression (corticosteroids, infliximab, mycophenolate), but may require treatment discontinuation. taVNS could potentially prevent or mitigate some immune-related adverse events while preserving anti-tumor immunity, given that vagal anti-inflammatory pathways act through different mechanisms than checkpoint molecules.

Stem Cell Transplantation and Cellular Therapies (Subcategory C): Hematopoietic stem cell transplantation (autologous or allogeneic) triggers inflammatory responses through conditioning regimens (chemotherapy and/or radiation), the infusion itself, and in allogeneic transplant, graft-versus-host disease. Cytokine release during neutrophil engraftment can cause fevers and organ dysfunction. Mesenchymal stem cell infusions and other cellular therapies can also provoke inflammatory responses. taVNS application in the peri-transplant period may reduce conditioning-related inflammation, mitigate cytokine release during engraftment, and potentially modulate graft-versus-host disease through effects on donor T cell activation and cytokine production.

Viral Vector Gene Therapy (Subcategory D): Gene therapy using adeno-associated virus (AAV), lentivirus, or other viral vectors can trigger innate and adaptive immune responses against the vector and/or transgene product. Inflammatory responses to viral vectors have caused severe and even fatal complications in clinical trials. The inflammatory response involves pattern recognition receptor activation by viral components, complement activation, cytokine release, and T cell responses against transduced cells. Pre-treatment and post-treatment with taVNS could potentially reduce vector immunogenicity and improve gene therapy safety and efficacy.

Vaccine Administration (Subcategory E): While vaccines are designed to stimulate protective immune responses, some vaccines (particularly those with adjuvants or novel platforms) can trigger significant inflammatory reactions. mRNA vaccines for COVID-19, for example, occasionally caused myocarditis in young males, likely through inflammatory mechanisms. Highly immunogenic vaccines or immunization schedules involving multiple concurrent vaccines could potentially benefit from peri-vaccination taVNS to reduce inflammatory adverse events while preserving immunogenicity.

Monoclonal Antibody Infusions (Subcategory F): Certain monoclonal antibody therapies, particularly those targeting immune cells or activating immune responses (such as blinatumomab, rituximab first infusion, or alemtuzumab) can cause cytokine release and infusion reactions. These reactions result from rapid immune cell activation, lysis, or engagement. Pre-treatment with anti-inflammatory medications is often employed, but taVNS could provide an alternative or complementary approach.

Organ Transplantation (Overlapping Subcategory): Organ transplantation represents a unique intervention that combines tissue-disruptive mechanisms (surgical trauma, ischemia-reperfusion injury) with immune-activating mechanisms (allogeneic immune responses, rejection, and paradoxically, immunosuppression-related infections that trigger inflammation). Transplantation also involves introduction of foreign material through vascular anastomoses and surgical materials. The multi-faceted inflammatory burden of transplantation makes it a particularly relevant application for taVNS, potentially reducing ischemia-reperfusion injury, surgical inflammation, and acute rejection episodes.

### Foreign-Material-Introducing Interventions (Meta-Category III)

Foreign-material-introducing interventions (1930) involve placement of non-biological materials or substances into the body, triggering foreign body responses, complement activation on material surfaces, and thromboinflammatory cascades. The inflammatory response to foreign materials is influenced by material composition, surface properties, size, location, and duration of contact with tissues or blood.

### Foreign-Material-Introducing Interventions Include Multiple Subcategories:

Implantable Medical Device Placement (Subcategory A): A wide range of medical devices are implanted for therapeutic purposes and elicit inflammatory responses. Examples include neurostimulators (spinal cord stimulators, deep brain stimulators, vagus nerve stimulators), cardiac devices (pacemakers, implantable cardioverter-defibrillators, cardiac resynchronization therapy devices, implantable loop recorders), orthopedic hardware (joint prostheses, spinal instrumentation, fracture fixation plates and screws), vascular devices (stents, grafts, vena cava filters, left atrial appendage occluders), and other implants (breast implants, penile prostheses, cochlear implants, intraocular lenses). The inflammatory response begins with protein adsorption to device surfaces, followed by neutrophil and macrophage recruitment, foreign body giant cell formation, and fibrous capsule development. Excessive inflammation can lead to device infection, fibrosis, malfunction, or rejection. taVNS applied in the peri-implantation period may reduce the intensity of the foreign body response and improve device integration.

Vascular Interventions (Subcategory B): Endovascular procedures including percutaneous coronary intervention with stent placement, peripheral artery stenting, and transcatheter valve replacement involve introduction of metallic or polymeric materials into the vascular system. These materials trigger contact activation of coagulation, complement activation, platelet adhesion and activation, and endothelial inflammatory responses. The thromboinflammatory response to vascular devices can lead to stent thrombosis, restenosis, or device-related embolic events. Antiplatelet and anticoagulant medications are standard but carry bleeding risks; taVNS could provide complementary anti-inflammatory and antithrombotic effects through endothelial stabilization and modulation of platelet-leukocyte interactions.

Contrast Agent Administration (Subcategory C): Iodinated contrast agents for computed tomography, gadolinium-based contrast agents for magnetic resonance imaging, and other imaging contrast media can trigger inflammatory and allergic-type reactions. Contrast-induced nephropathy (now termed contrast-associated acute kidney injury) involves inflammatory mechanisms including oxidative stress, tubular injury, and inflammatory mediator release. While some reactions are IgE-mediated allergies, others involve direct inflammatory activation. taVNS applied before contrast administration could potentially reduce the risk of contrast-associated complications, particularly in high-risk patients with chronic kidney disease, diabetes, or prior contrast reactions.

Blood Product Transfusion (Subcategory D): Transfusion of red blood cells, platelets, plasma, or other blood products can trigger inflammatory responses ranging from mild febrile reactions to severe transfusion-related acute lung injury (TRALI). TRALI involves antibody-mediated or bioactive lipid-mediated neutrophil activation in pulmonary capillaries, causing capillary leak and acute respiratory distress. Transfusion-associated circulatory overload (TACO) also has inflammatory components. Hemolytic transfusion reactions release inflammatory mediators. taVNS before or during transfusion could potentially reduce transfusion-related inflammatory complications.

Intra-Articular and Intrathecal Material Injection (Subcategory E): Injection of viscosupplements, corticosteroids, local anesthetics, or other materials into joints or the intrathecal space can trigger inflammatory reactions. Particulate corticosteroids, in particular, have caused severe inflammatory responses when inadvertently injected into neural tissue. Hyaluronic acid viscosupplements occasionally cause acute inflammatory flares in treated joints. Chemical meningitis can result from intrathecal introduction of certain substances. While these interventions are generally low-risk, taVNS could provide added protection in high-risk scenarios.

Dialysis and Apheresis (Subcategory F): Hemodialysis, peritoneal dialysis, and apheresis procedures involve blood contact with synthetic membranes and circuits, triggering complement activation, cytokine release, and chronic inflammatory state contributions. The bio-incompatibility of dialysis membranes is a recognized contributor to cardiovascular disease and mortality in dialysis patients. taVNS applied during or after dialysis sessions could potentially reduce dialysis-associated inflammation and improve long-term outcomes.

### Cross-Cutting Inflammatory Complication Classes

While each intervention category has characteristic complications, several inflammatory complication patterns occur across multiple intervention types. Understanding these cross-cutting patterns informs protocol design and biomarker selection.

Cytokine Release Syndrome (CRS): CRS represents a systemic inflammatory response characterized by fever, hypotension, capillary leak, and multi-organ dysfunction driven by massive cytokine elevation (particularly IL-6, IL-10, IFN-γ). CRS is most prominently associated with CAR-T cell therapy and certain monoclonal antibodies, but can also occur with severe infections, trauma, or other immune-activating interventions. Grading systems classify CRS severity from grade 1 (fever only) to grade 4 (life-threatening hypotension and hypoxia requiring intensive support). taVNS targets multiple mechanisms underlying CRS including IL-6 production through cholinergic anti-inflammatory pathway activation, endothelial stabilization to reduce capillary leak, and central anti-inflammatory network activation.

Immune Effector Cell-Associated Neurotoxicity Syndrome (ICANS): ICANS involves neurologic symptoms ranging from confusion and word-finding difficulty to seizures, cerebral edema, and coma. ICANS occurs predominantly with CAR-T therapy and other T cell-engaging immunotherapies. The pathophysiology involves blood-brain barrier disruption, inflammatory cytokine penetration into the central nervous system, and possibly direct T cell toxicity to brain endothelium. taVNS mechanisms relevant to ICANS prevention include central network suppression, reduced cerebral metabolism, microglial phenotype modulation, and blood-brain barrier stabilization through endothelial effects.

Systemic Inflammatory Response Syndrome (SIRS): SIRS represents a generalized inflammatory activation meeting criteria including fever or hypothermia, tachycardia, tachypnea, and leukocytosis or leukopenia. SIRS can result from infectious or non-infectious triggers including surgery, trauma, burns, pancreatitis, or ischemia-reperfusion injury. When SIRS progresses with organ dysfunction, it becomes sepsis (if infection-triggered) or multiple organ dysfunction syndrome (MODS). taVNS addresses SIRS through systemic anti-inflammatory cytokine modulation, autonomic stabilization, and interruption of inflammatory amplification cascades.

Acute Kidney Injury (AKI): AKI involves rapid decline in kidney function and can result from inflammatory mechanisms triggered by contrast agents, cardiopulmonary bypass, sepsis, drug toxicity, or ischemia-reperfusion injury. Inflammatory mediators contribute to tubular injury, vascular dysfunction, and impaired kidney recovery. AKI is associated with increased mortality and progression to chronic kidney disease. taVNS may protect against AKI through anti-inflammatory effects, improved renal perfusion via autonomic modulation, and reduction in oxidative stress.

Cerebral Edema: Cerebral edema involves brain swelling from vasogenic mechanisms (blood-brain barrier disruption with fluid extravasation), cytotoxic mechanisms (cellular swelling from energy failure), or mixed mechanisms. Cerebral edema complicates brain surgery, traumatic brain injury, stroke, ICANS, hepatic encephalopathy, and other conditions. Inflammatory mediators including matrix metalloproteinases, vascular endothelial growth factor (VEGF), and cytokines contribute to blood-brain barrier breakdown. taVNS mechanisms relevant to cerebral edema include central network suppression reducing metabolic demand, endothelial stabilization preserving blood-brain barrier integrity, and reduction in inflammatory mediator production.

Capillary Leak Syndrome: Capillary leak syndrome involves increased vascular permeability leading to fluid extravasation, hypoalbuminemia, hypotension, and tissue edema. Capillary leak can complicate CAR-T therapy, IL-2 therapy, sepsis, or transfusion reactions. Endothelial dysfunction and inflammatory mediator effects on endothelial tight junctions drive capillary leak. taVNS promotes endothelial stabilization through nitric oxide modulation, reduction in adhesion molecule expression, and suppression of cytokines that increase permeability.

Ischemia-Reperfusion Injury: Ischemia-reperfusion injury represents tissue damage occurring when blood flow is restored after a period of ischemia. The injury results from reactive oxygen species generation, complement activation, neutrophil infiltration, and inflammatory cytokine production during reperfusion. Ischemia-reperfusion injury affects the heart (myocardial infarction treatment), brain (stroke treatment), kidneys (transplantation), liver (transplantation, resection), and other organs. taVNS applied before, during, or immediately after reperfusion may reduce injury through cholinergic anti-inflammatory pathway activation, sympathetic withdrawal reducing metabolic demand, and modulation of neutrophil activation.

### Intervention-Specific Complication Profiles and Biomarker Patterns:

Different interventions within each category exhibit characteristic complication profiles and biomarker signatures that inform protocol selection and monitoring strategies.

For tissue-disruptive interventions, the inflammatory response typically manifests acutely within hours to days post-intervention, with biomarker patterns including elevations in C-reactive protein (CRP) beginning 6-12 hours post-surgery and peaking at 48-72 hours, interleukin-6 (IL-6) elevation within 2-6 hours peaking at 24 hours, and tumor necrosis factor-α (TNF-α) elevation in the immediate post-operative period. The magnitude of biomarker elevation generally correlates with surgical extent and tissue trauma. Complications such as SIRS manifest with fever, tachycardia, leukocytosis, and potentially organ dysfunction. Cerebral edema following neurosurgery may present with neurologic deterioration 24-72 hours post-operatively, with imaging confirmation. Acute kidney injury following cardiac surgery may manifest with rising creatinine and declining urine output within 48 hours.

For immune-activating interventions, particularly CAR-T therapy, cytokine release syndrome typically develops after a period of time e.g., 1-14 days post-infusion (most commonly 3-7 days), with biomarkers including IL-6 (often exceeding 1000 μg/mL in severe CRS, with some patients reaching >10,000 μg/mL), IL-10, IFN-γ, ferritin (often >10,000 ng/mL), CRP, and procalcitonin. Clinical manifestations include fever (often the earliest sign), hypotension progressing to shock, hypoxia requiring supplemental oxygen or ventilatory support, and multi-organ dysfunction. ICANS may occur concurrently with or following CRS, typically 4-10 days post-infusion, with manifestations including confusion, aphasia, tremor, seizures, or decreased level of consciousness. Grading systems for both CRS and ICANS guide clinical management decisions.

For immune checkpoint inhibitor therapy, immune-related adverse events can develop at any time during treatment or even after discontinuation, though typically occur within weeks to months of initiation. Different organ systems may be affected with varying time courses: colitis often develops within 2-3 months, hepatitis within 2-4 months, pneumonitis within 2-6 months, and endocrinopathies potentially later. Biomarkers are often organ-specific (elevated liver enzymes for hepatitis, elevated thyroid stimulating hormone for thyroiditis) rather than generalized inflammatory markers, though some patients exhibit elevated inflammatory markers.

For foreign-material-introducing interventions, the inflammatory time course varies with material type and location. Vascular stent placement triggers immediate thromboinflammatory responses with platelet activation, complement activation, and cytokine release within hours, with inflammatory biomarker elevation typically modest unless complications occur. Late stent thrombosis can occur months to years later. Implantable device placement triggers an initial acute inflammatory response over days to weeks with elevation in CRP and local inflammatory mediators, followed by chronic foreign body reaction over months with fibrous capsule formation. Device infections may present acutely or in delayed fashion with systemic inflammatory markers and local signs.

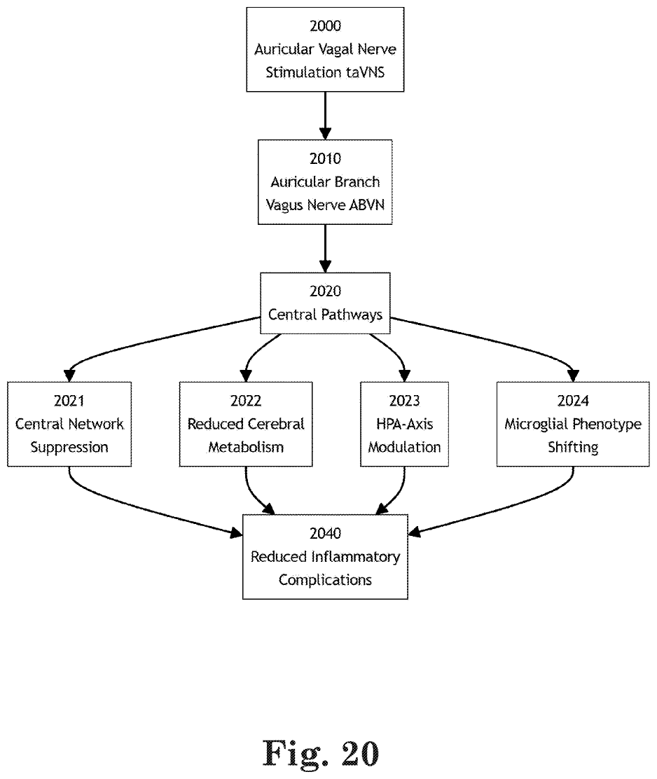

FIG. 20 illustrates central nervous system mechanisms activated by taVNS. Auricular vagal nerve stimulation 2000 activates the auricular branch of the vagus nerve (ABVN) 2010, which projects to central pathways 2020 in the nucleus tractus solitarius of the brainstem and higher cortical structures including the insula, orbitofrontal cortex, and other components of the central autonomic network.

Central Network Suppression (2021): taVNS-induced modulation of the central autonomic network activates two distinct but complementary mechanisms of central anti-inflammatory control. First, at sub-perceptive amplitudes (approximately 0.05 mA to 0.5 mA), taVNS preferentially suppresses high-gamma neural activity (approximately 60-150 Hz) in the insular cortex and orbitofrontal cortex (OFC) without requiring conscious sensory perception of the stimulation. The insular cortex functions as an interoceptive integration hub that receives afferent signals about the body's internal state and regulates downstream autonomic, immune, and affective responses. High-gamma oscillations in the insula reflect active inflammatory drive and sympathetic output; their suppression by sub-perceptive taVNS reduces regional metabolic demand in the insula and OFC and attenuates their output to downstream sympathetic and inflammatory pathways. This high-gamma suppression mechanism constitutes a distinct central autonomic/limbic gateway modulation pathway that is separable from and complementary to peripheral cholinergic anti-inflammatory pathway activation. Because this mechanism does not depend on conscious sensory processing or peripheral reflex activation, it may be active even during general anesthesia, deep sedation, or in patients with impaired consciousness, making it particularly relevant for intra-operative and intensive care applications. Second, at moderate and higher amplitudes, taVNS-induced modulation of the central autonomic network suppresses excessive sympathetic outflow and reduces central drive for inflammatory responses through broader network-level effects. Hyperactivation of insular cortex has been associated with increased inflammatory cytokine production and exaggerated stress responses; taVNS suppresses insular overactivation, thereby reducing centrally-mediated inflammatory drive. This mechanism is particularly relevant for inflammatory responses that have significant central nervous system components, including stress-induced inflammation, neuroinflammation associated with surgery or trauma, and modulation of fever responses. Functional neuroimaging studies have demonstrated that taVNS modulates activity in the insula, anterior cingulate cortex, and other limbic structures involved in autonomic and immune regulation. The sub-perceptive high-gamma suppression mechanism and the broader amplitude-dependent central network suppression mechanism thus represent two distinct mechanistic nodes within the central autonomic/limbic layer, providing multiple orthogonal pathways by which taVNS reduces centrally-mediated inflammation across the peri-intervention period.

Reduced Cerebral Metabolism (2022): taVNS induces changes in regional cerebral blood flow and glucose metabolism, particularly in limbic and autonomic regulatory regions. This metabolic suppression may have protective effects against cerebral edema and excitotoxicity in contexts such as post-operative delirium, stroke, traumatic brain injury, or neurotoxicity from immunotherapies such as CAR-T cells causing ICANS. The reduction in cerebral metabolic demand may also reduce production of reactive oxygen species and inflammatory mediators by brain cells.

HPA-Axis Modulation (2023): taVNS affects the hypothalamic-pituitary-adrenal (HPA) axis, which regulates cortisol release and systemic stress responses. The HPA axis has complex bidirectional interactions with immune function: moderate cortisol elevation provides anti-inflammatory effects through glucocorticoid receptor activation, but excessive or prolonged cortisol can cause immunosuppression and metabolic derangements. taVNS can modulate HPA axis activity, potentially optimizing the anti-inflammatory effects of endogenous glucocorticoids while preventing excessive immunosuppression.

Microglial Phenotype Shifting (2024): Microglia, the resident immune cells of the central nervous system, exhibit phenotypic plasticity ranging from pro-inflammatory (classically activated, M1-like) to anti-inflammatory/reparative (alternatively activated, M2-like) states. taVNS can promote microglial phenotype shifting toward M2-like states characterized by production of anti-inflammatory mediators including IL-10 and TGF-β, reduced production of pro-inflammatory cytokines and reactive oxygen species, and enhanced phagocytosis of debris with reduced bystander tissue damage. This mechanism is particularly relevant for interventions affecting the central nervous system, including neurosurgery, radiation therapy to the brain or spine, ICANS from immunotherapy, and systemic inflammatory conditions with CNS manifestations.

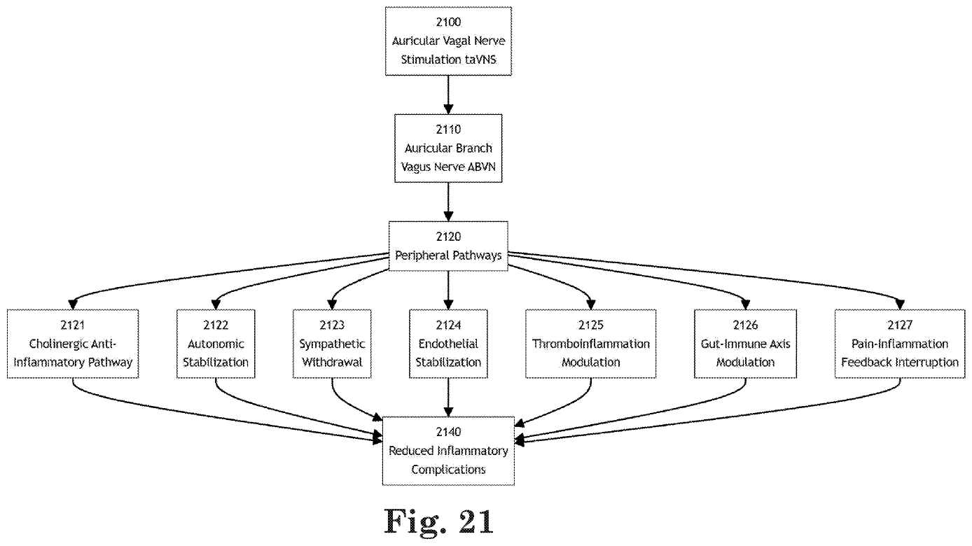

These central mechanisms converge to produce reduced inflammatory complications 2040, working in concert with peripheral mechanisms described in FIG. 21.

FIG. 21 illustrates peripheral mechanisms activated by taVNS. Auricular vagal nerve stimulation 2100 activates ABVN 2110, which projects to central structures that in turn activate efferent vagal pathways constituting peripheral pathways 2120.

Cholinergic Anti-Inflammatory Pathway (CAP) (2121): The cholinergic anti-inflammatory pathway is a well-characterized neuroimmune mechanism activated by taVNS through the following molecular cascade: taVNS activates auricular vagal afferent fibers projecting to the nucleus tractus solitarius, which in turn activates efferent vagal pathways including splenic sympathetic nerve endings (2128); these splenic sympathetic fibers stimulate a subset of splenic T cells to synthesize and release acetylcholine (2129); the released acetylcholine binds to alpha-7 nicotinic acetylcholine receptors (α7 nAChR) expressed on macrophages, microglia, dendritic cells, and other immune effector cells (2130); α7 nAChR activation recruits the JAK2-STAT3 intracellular signaling cascade, which suppresses nuclear factor-κB (NF-κB) nuclear translocation (2131), thereby reducing transcription and production of pro-inflammatory cytokines including tumor necrosis factor-α (TNF-α), interleukin-10 (IL-1β), and interleukin-6 (IL-6), while promoting production of anti-inflammatory cytokines such as interleukin-10 (IL-0). Additional protective effects include preservation of blood-brain barrier integrity through reduction of inflammatory endothelial activation. The α7 nAChR/NF-κB/JAK2-STAT3 molecular pathway has been demonstrated to reduce inflammation in preclinical models of sepsis, endotoxemia, ischemia-reperfusion injury, and other conditions. The pathway is particularly important in splenic macrophages, but α7 nAChR are also expressed on tissue macrophages in multiple organ systems, dendritic cells, and some T cell populations. This mechanism is broadly relevant across all three intervention categories as macrophage activation and NF-κB-driven cytokine production are common features of tissue injury, immune activation, and foreign body responses.

Autonomic Stabilization (2122): taVNS modulates the balance between sympathetic and parasympathetic nervous system activity to achieve autonomic stabilization. Increased vagal tone reduces sympathetic overdrive, with downstream effects including reduced catecholamine release, reduced inflammatory signaling through adrenergic receptors on immune cells, improved heart rate variability (HRV), and stabilization of hemodynamic parameters. Autonomic stabilization through taVNS is measurable via HRV metrics including SDNN and RMSSD, which reflect the degree of parasympathetic restoration achieved.

Sympathetic Withdrawal (2123): As a complementary mechanism to autonomic stabilization, taVNS promotes sympathetic withdrawal, reducing norepinephrine-driven activation of immune cells through β-adrenergic receptors. Sympathetic withdrawal attenuates the adrenergic amplification of inflammatory cytokine production, reduces oxidative stress associated with catecholamine excess, and limits the stress-hormone-driven inflammatory cascade that is particularly active during surgical and procedural interventions. Sympathetic withdrawal and cholinergic anti-inflammatory pathway activation operate through distinct but complementary molecular mechanisms and are additive in their anti-inflammatory effect.

Endothelial Stabilization (2124): taVNS promotes endothelial stabilization through multiple mechanisms, including increased nitric oxide production by endothelial nitric oxide synthase (eNOS), suppression of endothelial adhesion molecule expression (ICAM-1, VCAM-1, E-selectin), and reduced endothelial permeability. These effects reduce leukocyte trafficking to sites of injury and attenuate capillary leak. Endothelial stabilization is particularly relevant for preventing capillary leak syndrome, reducing edema formation, and preserving organ perfusion during inflammatory cascades associated with tissue-disruptive, immune-activating, and foreign-material-introducing interventions. Von Willebrand factor (vWF) and angiopoietin-2 serve as biomarkers of endothelial activation and may be used to assess the degree of endothelial stabilization achieved.

Thromboinflammation Modulation (2125): taVNS modulates thromboinflammatory responses including platelet activation, coagulation cascade activation, and complement-mediated endothelial injury. Thromboinflammation—the pathologic intersection of coagulation and inflammatory pathway activation—is a prominent feature of ischemia-reperfusion procedures, vascular device implantation, cardiopulmonary bypass, and foreign-material-introducing interventions. Through autonomic modulation and endothelial stabilization, taVNS reduces the pro-thrombotic and pro-inflammatory endothelial phenotype that drives thromboinflammatory cascades. Von Willebrand factor (vWF) and D-dimer serve as biomarkers of thromboinflammatory modulation by taVNS.

Gut-Immune Axis Modulation (2126): taVNS engages the gut-immune axis through modulation of enteric nervous system activity and gut microbiome composition, reducing translocation of bacterial products and inflammatory mediators from the gut lumen into the systemic circulation. The vagus nerve provides direct innervation to the gastrointestinal tract and modulates intestinal motility, mucosal barrier integrity, and local immune cell activity. Gut-immune axis modulation is particularly relevant for abdominal surgery, immunotherapy interventions where gut barrier integrity is compromised, and critical illness states where gut translocation contributes to systemic inflammation. Lipopolysaccharide-binding protein (LBP) and calprotectin may serve as biomarkers of gut-immune axis engagement.

Pain-Inflammation Feedback Loop Interruption (2127): taVNS interrupts the bidirectional pain-inflammation feedback loop through analgesic effects that reduce pain signal transmission and perception, and anti-inflammatory effects that reduce inflammatory mediator production at peripheral tissue sites. Uncontrolled pain generates sympathetic activation and stress hormone release that amplify inflammatory responses; by interrupting this feedback loop, taVNS reduces both the pain experience and the inflammatory amplification that pain-driven sympathetic activation produces. This mechanism also reduces opioid requirements that would otherwise contribute to immunosuppression and delayed recovery. Pain-inflammation feedback loop interruption is particularly relevant for post-operative and post-procedural recovery phases.

These peripheral mechanisms contribute to reduced inflammatory complications 2140, complementing the central mechanisms illustrated in FIG. 20.

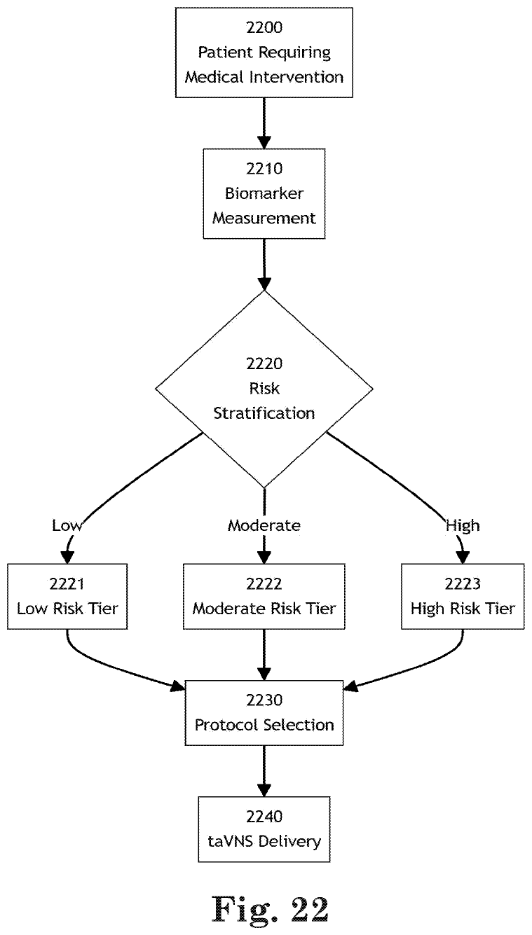

FIG. 22 illustrates a systematic approach to patient risk stratification and protocol selection. The process begins with patient requiring medical intervention 2200, followed by biomarker measurement 2210 to assess baseline inflammatory status and physiologic reserve.

Biomarker Measurement and Assessment (2210): Biomarker measurement encompasses inflammatory markers, autonomic function indicators, and organ function parameters. Key baseline assessments include:

C-reactive protein (CRP) as a marker of chronic low-grade inflammation, with normal values typically <3 mg/L, borderline elevation 3-10 mg/L, and significant elevation >10 mg/L. Elevated baseline CRP predicts increased risk of post-operative complications and may reflect underlying inflammatory conditions.

Interleukin-6 (IL-6) as a more sensitive marker of active inflammation, with normal values typically <5 μg/mL. Baseline IL-6 elevation may indicate active inflammatory processes or vulnerability to exaggerated inflammatory responses.

Tumor necrosis factor-α (TNF-α) as an early inflammatory mediator, though less commonly measured in routine practice due to assay variability and rapid fluctuations.

Ferritin, which can reflect both iron stores and inflammation (as an acute phase reactant). Very elevated ferritin (>500-1000 ng/mL) in the absence of iron overload disorders suggests inflammatory activity.

Additional biomarkers relevant to specific intervention categories include calprotectin, a neutrophil-derived inflammation marker relevant to gut-immune axis monitoring in abdominal surgery and immune checkpoint inhibitor-associated colitis; neutrophil-to-lymphocyte ratio (NLR), a composite hematologic marker of systemic inflammatory activation available from standard complete blood counts and useful in preconditioning patient characterization; interleukin-8 (IL-8), relevant to transfusion-related acute lung injury monitoring and post-operative inflammation; interleukin-2 (IL-2), relevant to organ transplantation and allogeneic immune response monitoring; donor-specific antibodies (DSA), markers of alloimmune activation relevant to organ transplantation rejection; lipopolysaccharide-binding protein (LBP), a gut-immune axis marker relevant to major abdominal surgery and postoperative ileus; intercellular adhesion molecule-1 (ICAM-1) and vascular cell adhesion molecule-1 (VCAM-1), endothelial adhesion molecule markers of sympathovagal imbalance and endothelial activation that may be monitored as biomarkers of taVNS-mediated endothelial stabilization.

Heart rate variability (HRV) as a non-invasive measure of autonomic function and vagal tone. HRV metrics including standard deviation of NN intervals (SDNN), root mean square of successive differences (RMSSD), and low-frequency/high-frequency ratio (LF/HF ratio) provide information about autonomic balance. Low HRV (for example, SDNN <50 ms in a 24-hour recording) indicates reduced autonomic reserve and is associated with increased cardiovascular risk, inflammation, and poor surgical outcomes.

Blood pressure variability, which when excessive may indicate autonomic dysfunction and inflammatory vascular changes.

Additional patient factors considered in risk assessment include age (older patients generally have higher inflammatory responses and reduced physiologic reserve), comorbidities (diabetes, obesity, chronic kidney disease, autoimmune diseases, and cardiovascular disease increase inflammatory risk), nutritional status (malnutrition impairs immune function and wound healing), prior history of inflammatory complications or exaggerated responses to interventions, genetic factors if known (polymorphisms in cytokine genes or pattern recognition receptors may influence inflammatory responses), and current medications (immunosuppressive medications, anti-inflammatory medications, or medications that affect autonomic function).

Intervention-specific factors include the complexity and duration of the planned intervention, degree of expected tissue trauma, whether ischemia-reperfusion is anticipated, immunogenicity of biologics or cellular therapies, foreign material type and quantity, and institutional experience and outcomes data.

Risk Stratification (2220): Based on the comprehensive assessment, patients are assigned to risk tiers. Risk stratification 2220 yields assignment to low risk tier 2221, moderate risk tier 2222, or high risk tier 2223.

Low risk tier 2221 includes patients with normal baseline inflammatory markers (CRP <3 mg/L, IL-6<5 μg/mL), good autonomic function (SDNN>100 ms), minimal comorbidities, and undergoing low-complexity interventions with expected minimal tissue trauma and short duration. Examples might include simple foreign material introduction (joint injection, peripheral IV contrast administration) or minor surgical procedures in healthy patients. These patients have low baseline inflammatory tone and low expected inflammatory burden from the intervention.

Moderate risk tier 2222 includes patients with mildly elevated inflammatory markers (CRP 3-10 mg/L, IL-6 5-20 pg/mL), modestly reduced autonomic function (SDNN 50-100 ms), some comorbidities (well-controlled diabetes, obesity, controlled hypertension), and/or undergoing moderate-complexity interventions. Examples might include major surgery in relatively healthy patients, moderate-risk immunotherapy in patients without baseline immune activation, or device implantation with moderate inflammatory potential. These patients have either elevated baseline risk or elevated intervention risk, but not both at extreme levels.

High risk tier 2223 includes patients with significantly elevated inflammatory markers (CRP >10 mg/L, IL-6 >20 μg/mL), poor autonomic function (SDNN <50 ms), multiple comorbidities or severe comorbidities (poorly controlled diabetes, morbid obesity, chronic kidney disease stage 4-5, active autoimmune disease), and/or undergoing high-risk interventions. Examples include major surgery in frail or multi-morbid patients, CAR-T cell therapy in patients with high tumor burden and baseline inflammatory activation, organ transplantation, or combinations of risk factors. These patients have high likelihood of developing significant inflammatory complications without intervention.

Some classification systems may employ four or five tiers for more granular stratification, or may use numerical scoring systems that weight different risk factors. The three-tier system provides a practical framework for protocol selection while allowing clinical judgment to modify assignments in borderline cases.

Protocol Selection (2230): Protocol selection 2230 matches the risk tier to an appropriate taVNS protocol, considering: preconditioning duration and timing (ranging from single 2-4 hour sessions for low-risk to 3-7 days for high-risk patients); stimulation intensity parameters (amplitude, pulse width, frequency, duty cycle) scaled to risk level; intra-intervention application approach (continuous versus intermittent, synchronization with procedural phases); post-intervention duration (24-72 hours for low-risk to 7-14+ days for high-risk); whether adaptive parameter adjustment based on biomarker feedback is employed; and integration with other anti-inflammatory interventions versus stand-alone use taVNS Delivery (2240): taVNS delivery 2240 executes the selected protocol with ongoing monitoring of clinical status, inflammatory biomarkers, autonomic function, and adverse effects. The delivery phase involves device placement, confirmation of adequate stimulation, adherence to prescribed parameters, and monitoring for adverse effects. Protocols should allow dynamic adjustment based on patient response, with escalation if unexpected severe inflammation develops or de-escalation if the patient tolerates the intervention better than anticipated

### Parameter Adjustment and Stimulation Protocols:

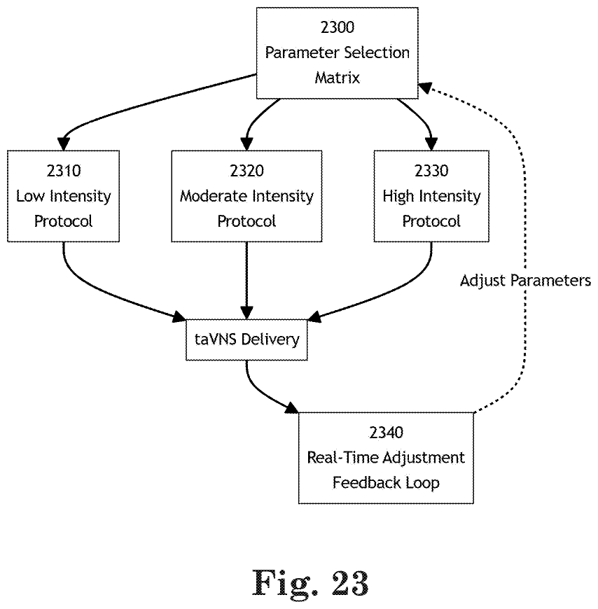

FIG. 23 illustrates the relationship between patient/intervention characteristics and taVNS parameter selection. The parameter selection matrix 2300 provides a framework for choosing from low intensity protocol 2310, moderate intensity protocol 2320, or high intensity protocol 2330, with real-time adjustment feedback loop 2340 allowing dynamic optimization.

Low Intensity Protocol (2310): Low intensity protocol 2310 employs gentler stimulation parameters appropriate for low-risk patients, initial preconditioning phases, or maintenance therapy during recovery phases. Typical parameters include:

Current amplitude in the range of 0.05-1.0 mA ranges from sub-perceptive to mildly perceptible. Sub-perceptive amplitudes typically range from about 0.05 mA to about 0.5 mA, though the specific threshold varies between individuals based on factors including skin thickness, electrode contact quality, and individual sensory sensitivity. At the lower end of this range (0.1-0.5 mA), sub-perceptive stimulation may preferentially activate central pathways with minimal or no peripheral sensation. At the upper end (0.5-1.0 mA), a mild tingling sensation may be perceived but remains comfortable for extended wear. Sub-perceptive stimulation is particularly useful for continuous or extended-duration protocols where patient comfort is paramount, and for targeting central network suppression mechanisms that may be activated even in the absence of conscious sensory perception.

Pulse width in the range of 50-250 microseconds, which is sufficient to activate nerve fibers while minimizing charge delivery per pulse.

Frequency in the range of 1-25 Hz, encompassing both low-frequency stimulation (1-10 Hz) thought to preferentially activate parasympathetic pathways, and moderate frequencies (10-25 Hz) that may provide balanced activation of anti-inflammatory mechanisms.

Duty cycle of 5-30%, meaning the stimulation is on for only a small fraction of time, with substantial off-periods. For example, a 10% duty cycle might involve 6 seconds on, 54 seconds off in each minute. This intermittent pattern is generally well-tolerated for extended periods and may prevent habituation or accommodation.

Session duration of 10-60 minutes per session, which is sufficient to activate anti-inflammatory pathways without requiring extended device wear.

Session frequency of once or twice daily, providing regular anti-inflammatory stimulation while minimizing device placement time and allowing for normal activities between sessions.

This protocol is appropriate for low-risk patients undergoing minor interventions, for initial preconditioning in the days before higher-risk interventions (to prime anti-inflammatory responses without inducing excessive vagal activation), or for maintenance therapy after the acute inflammatory period has resolved but some ongoing risk remains.

Moderate Intensity Protocol (2320): Moderate intensity protocol 2320 provides more robust anti-inflammatory stimulation appropriate for moderate-risk patients or active management of developing inflammatory responses. Typical parameters include:

Current amplitude in the range of 0.5-4.0 mA, producing mild to moderate sensation. Most patients tolerate 1-3 mA comfortably when awake, with perception of tingling or pulsing sensation in the ear that is not painful. Amplitude toward the higher end of this range (3-4 mA) may be used during anesthesia or in patients with higher sensory thresholds.

Pulse width in the range of 100-500 microseconds, providing greater charge delivery per pulse than low intensity protocols and potentially recruiting more nerve fibers or achieving greater depth of activation.

Frequency in the range of 10-50 Hz, which encompasses frequencies shown in various studies to activate anti-inflammatory pathways. Some protocols use 25 Hz as a standard frequency, while others employ higher frequencies (30-50 Hz) for more intensive effects.

Duty cycle of 25-50%, providing balanced on/off cycling. For example, a 50% duty cycle alternates equal periods on and off (such as 30 seconds on, 30 seconds off), while 25% duty cycle provides 15 seconds on, 45 seconds off. The increased on-time compared to low intensity protocols delivers more total stimulation.

Session duration of 30 minutes to 4 hours, allowing for extended stimulation during critical periods such as surgery, the immediate post-operative period, or active inflammatory complications. Some protocols employ continuous or near-continuous stimulation (18-24 hours per day) at moderate intensity in high-risk periods.

Session frequency of 2-4 times daily in intermittent protocols, or continuous/near-continuous application during high-risk periods.

This protocol is appropriate for moderate-risk patients throughout the peri-intervention period, for intra-operative application during surgery, for initial management of developing inflammatory complications before escalating to high intensity, or as maintenance following de-escalation from high intensity protocols.

High Intensity Protocol (2330): High intensity protocol 2330 delivers maximal anti-inflammatory stimulation for high-risk patients or active management of severe inflammatory complications. Typical parameters include:

Current amplitude in the range of 1.0-10 mA, with most patients tolerating 4-8 mA while awake (at the border of discomfort but below pain threshold), and up to 10 mA potentially used during anesthesia or deep sedation. Some patients may not tolerate the upper end of this range while conscious; in such cases, the maximum tolerable amplitude is used.

Pulse width in the range of 200-2000 microseconds, providing substantial charge delivery. Longer pulse widths (500-2000 s) may recruit deeper or larger-diameter nerve fibers and provide more robust activation of anti-inflammatory pathways.

Frequency in the range of 25-100 Hz, with some protocols employing frequencies of 30-50 Hz as standard high-intensity settings, while others explore higher frequencies up to 100 Hz for maximal effect. Very high frequencies (>50 Hz) may be less tolerable in conscious patients due to intensity of sensation.

Duty cycle of 40-75%, meaning stimulation is on the majority of the time with only brief rest periods. For example, 75% duty cycle might provide 45 seconds on, 15 seconds off. Some protocols employ continuous (100% duty cycle) stimulation during critical periods, though this may increase risk of accommodation and sensory discomfort.

Session duration of 1-24 hours, including continuous stimulation during critical periods such as active cytokine release syndrome management, the acute post-operative period after high-risk surgery, or during and immediately after ischemia-reperfusion procedures. Extended continuous stimulation requires attention to device comfort, skin integrity, and patient tolerance.

Session frequency of continuous or multiple times per day (4-6 sessions daily) when using intermittent protocols.

This protocol is appropriate for high-risk patients throughout the peri-intervention period, for active management of inflammatory complications such as cytokine release syndrome or ICANS, for synchronized application during reperfusion in ischemia-reperfusion procedures, or for salvage therapy when moderate intensity protocols prove insufficient.

In extended-duration taVNS protocols, repetitive delivery of a fixed periodic waveform may result in neural habituation and accommodation, reducing the effectiveness of stimulation over time. To reduce habituation and accommodation, taVNS may be delivered using a stochastically modulated waveform in which at least one stimulation parameter selected from current amplitude, pulse width, frequency, and/or inter-pulse interval is varied in a pseudo-random or noise-modulated manner within a therapeutically effective range. Stochastic variation introduces temporal irregularity that approximates the natural aperiodic firing patterns of biological neural systems, reducing the tendency of target afferent populations to adapt to a predictable periodic stimulus. The degree of stochastic variation may be bounded within a defined range for example, varying amplitude by ±20% around a central set point, or varying inter-pulse interval according to a defined probability distribution to maintain individual parameter values within therapeutically effective bounds while preserving temporal unpredictability. Stochastic waveform modulation is particularly advantageous for continuous intraoperative delivery and for multi-day post-intervention protocols involving extended session durations.

Real-Time Adjustment Feedback Loop (2340): The real-time adjustment feedback loop 2340 allows parameters to be modified based on physiologic indicators and clinical response. Feedback mechanisms include:

Heart rate variability monitoring, with increasing HRV (particularly increased high-frequency power or RMSSD) indicating effective vagal activation. If HRV does not improve or worsens, parameters may be adjusted (typically increasing intensity) to achieve better autonomic modulation. Conversely, if excessive bradycardia occurs (heart rate <50 bpm in patients without baseline bradycardia), intensity may be reduced.

Inflammatory biomarker measurement at intervals during treatment, with trends in IL-6, CRP, and other markers guiding adjustments. For example, if a patient receiving moderate intensity stimulation shows persistent or rising IL-6 levels 24-48 hours into treatment, escalation to high intensity may be warranted. Conversely, rapidly declining biomarkers may allow de-escalation.

Clinical status including fever patterns, hemodynamic stability, oxygen requirements, mental status, organ function trends, and overall trajectory. Clinical improvement may allow protocol de-escalation, while deterioration may prompt escalation or addition of pharmacologic interventions.

Adverse effects including skin irritation at electrode sites, discomfort or pain from stimulation, or systemic effects (though serious adverse effects from taVNS are rare). If adverse effects occur, parameters may be adjusted (typically reducing amplitude or duty cycle) to improve tolerance while maintaining therapeutic benefit.

The feedback loop recognizes that individual responses to taVNS vary based on factors including baseline autonomic tone, severity of inflammation, concurrent interventions, and inter-individual variability in nerve anatomy and sensitivity. Adaptive protocols that incorporate feedback may achieve better outcomes than rigid protocols, particularly in heterogeneous high-risk populations.

Timing and Multi-Phase Protocols: The timing of taVNS delivery relative to the medical intervention is a critical determinant of efficacy. Three phases can be distinguished, each with distinct therapeutic objectives and potentially different optimal parameters.

Pre-Intervention Phase (Preconditioning): Stimulation delivered hours to days before the intervention may “precondition” the immune system to mount a more controlled inflammatory response. The concept of preconditioning is well-established in ischemic preconditioning, where brief ischemic episodes protect against subsequent prolonged ischemia. Analogously, taVNS preconditioning may prepare anti-inflammatory pathways for activation during the intervention.

Mechanisms of taVNS preconditioning may include inducing anti-inflammatory cytokine production, particularly interleukin-10 (IL-10), which can persist after stimulation ceases and provide ongoing anti-inflammatory effects; promoting M2 macrophage polarization such that tissue macrophages are primed toward anti-inflammatory rather than pro-inflammatory responses when the intervention occurs; enhancing HRV and autonomic reserve, providing greater capacity to maintain autonomic balance during the stress of the intervention; reducing baseline inflammatory tone in high-risk patients who present with elevated inflammatory markers, thereby lowering the starting point from which intervention-induced inflammation must escalate; and potentially inducing adaptive cellular responses such as heat shock protein expression or antioxidant enzyme upregulation that provide cytoprotection.

Duration and intensity of preconditioning may vary based on risk stratification. Low-risk patients may receive a single session 2-4 hours before the intervention, using low to moderate intensity parameters (0.5-2 mA, 200-300 s, 20-25 Hz, 30% duty cycle, 30-60 minutes). This brief preconditioning primes anti-inflammatory pathways without extensive treatment burden.

Moderate-risk patients may receive preconditioning for 1-3 days before the intervention, with daily or twice-daily sessions using moderate intensity parameters (1-3 mA, 250-400 s, 25-30 Hz, 40% duty cycle, 45-90 minutes per session). This extended preconditioning allows for more robust priming of anti-inflammatory mechanisms.

High-risk patients may receive preconditioning for 3-7 days before the intervention, with twice-daily or more frequent sessions using moderate to high intensity parameters (2-4 mA, 300-500 s, 25-40 Hz, 50% duty cycle, 60-120 minutes per session). In extremely high-risk cases, preconditioning might extend even longer, analogous to intensive preparation before high-risk surgery.

Preconditioning is particularly applicable to scheduled interventions where timing is controlled. Emergency interventions may not allow for extended preconditioning, though even brief preconditioning (30-60 minutes immediately before) may provide some benefit.

Intra-Intervention Phase: Continuous or intermittent stimulation during the intervention may limit the magnitude of inflammatory mediator release triggered by tissue trauma, ischemia, or immune activation. This represents “real-time” anti-inflammatory modulation coinciding with the inflammatory trigger.

Mechanisms during the intra-intervention phase include cholinergic anti-inflammatory pathway activation suppressing cytokine production by macrophages as they encounter DAMPs or other inflammatory stimuli; central network suppression reducing central drive for stress responses and inflammatory activation during the procedure; autonomic stabilization maintaining hemodynamic stability and reducing sympathetic surge; endothelial stabilization during the procedure potentially reducing the degree of endothelial activation and capillary leak that develops; and in ischemia-reperfusion procedures, synchronized stimulation during reperfusion may provide maximal protection against reperfusion injury.

Practical considerations for intra-intervention stimulation include device placement before anesthesia induction to ensure correct positioning while the patient is awake and can provide feedback, though some devices can be placed on anesthetized patients using anatomical landmarks. Device compatibility with surgical site, anesthesia monitoring equipment, and other procedural equipment must be verified. Electromagnetic interference with electrocautery or other surgical equipment is generally not problematic with modern devices, though testing in specific environments is prudent. Remote monitoring and control capabilities allow anesthesia providers or other clinicians to adjust parameters, check device function, or turn the device off if needed without disturbing the surgical field.

Parameter selection during the intervention typically uses moderate to high intensity to provide robust anti-inflammatory effects: 2-6 mA amplitude (higher amplitudes acceptable during general anesthesia when patient sensation is not a concern), 250-500 s pulse width, 25-50 Hz frequency, 50-75% duty cycle or continuous, and duration throughout the procedure.

For ischemia-reperfusion procedures, stimulation may be synchronized with the reperfusion phase, with initiation or intensification of stimulation timed to begin just before or coinciding with restoration of blood flow (for example, aortic de-clamping in cardiac surgery, or opening of an occluded artery in stroke thrombectomy). Burst mode stimulation (brief periods of very high frequency stimulation) might be delivered at the moment of reperfusion to provide maximal pathway activation during the critical injury period.

Post-Intervention Phase: Stimulation after the intervention addresses ongoing inflammatory responses, prevents progression to severe complications, and accelerates resolution of inflammation and tissue repair.

Mechanisms in the post-intervention phase include suppression of ongoing inflammatory cytokine production by activated immune cells, enhancement of anti-inflammatory and pro-resolution mediators (IL-10, TGF-β, specialized pro-resolving mediators), acceleration of the transition from neutrophil-dominated inflammation to macrophage-mediated resolution and repair, prevention of secondary complications such as infection (through appropriate immune modulation rather than immunosuppression), post-operative atelectasis and pneumonia (through improved autonomic function and reduced systemic inflammation), or delayed inflammatory complications (such as late-onset ICANS after CAR-T therapy), reduction in post-operative pain and opioid requirements through both anti-inflammatory and direct analgesic effects, and improvement in functional recovery and rehabilitation through reduced inflammatory interference with healing.

Duration of post-intervention treatment varies by risk tier and intervention type. Low-risk interventions may require only 24-72 hours of post-intervention treatment with moderate intensity parameters, administered as 2-3 sessions daily. Moderate-risk interventions may require 3-7 days of treatment, with moderate to high intensity parameters, administered as 2-4 sessions daily or continuous application during the first 24-48 hours followed by intermittent sessions. High-risk interventions or development of complications may require 7-14 days or longer of treatment, with high intensity parameters initially, administered continuously or nearly continuously (18-24 hours/day) during the acute phase (first 3-5 days), then transitioning to intermittent sessions (2-4 times daily) as inflammation resolves.

The transition from post-intervention treatment to discontinuation should be guided by biomarker normalization (CRP returning toward normal, IL-6<10 μg/mL), clinical stability (afebrile, hemodynamically stable, improving organ function), and re-establishment of normal autonomic function (HRV metrics normalizing). Abrupt discontinuation may be appropriate in patients doing well with minimal inflammatory response, while gradual tapering (reducing intensity parameters and/or session frequency over several days) may be preferable in patients who had severe inflammation to avoid rebound effects.

Different parameters may be optimal for different phases based on the therapeutic objectives. Pre-intervention preconditioning might employ moderate intensity with emphasis on autonomic optimization (frequencies around 20-25 Hz, amplitude 1-2 mA, moderate duty cycles), delivered intermittently to allow physiologic adaptation without exhausting responses. Intra-intervention application might employ higher intensity to provide robust acute anti-inflammatory effects during the peak inflammatory trigger (amplitude 3-6 mA, frequencies 25-50 Hz, high duty cycles or continuous). Post-intervention early phase (first 24-72 hours) might employ high intensity to suppress acute inflammatory responses (amplitude 2-6 mA depending on tolerance, frequencies 25-40 Hz, high duty cycles), while post-intervention later phase (after 72 hours) might transition to moderate intensity as inflammation resolves (amplitude 1-3 mA, frequencies 20-30 Hz, moderate duty cycles).

These phase-specific adjustments recognize that the inflammatory response is dynamic, with different mechanisms predominating at different times, and that the balance between efficacy and tolerability may shift across phases (greater tolerance for intensive parameters during anesthesia or critical illness, preference for comfortable parameters during recovery when quality of life becomes more salient).

### Closed-Loop Adaptive Systems:

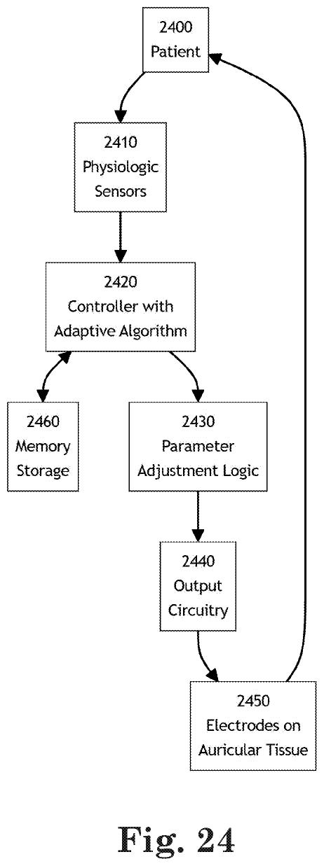

FIG. 24 illustrates an adaptive taVNS system that continuously optimizes therapy based on physiologic feedback. This represents an advanced implementation that may improve outcomes through individualized, dynamic parameter adjustment.

Patient (2400) and Physiologic Sensors (2410): Patient 2400 is monitored via physiologic sensors 2410, which may include electrocardiogram (ECG) sensors for continuous heart rate variability (HRV) analysis, with metrics calculated in real-time or at regular intervals (such as 5-minute windows). HRV analysis provides information about autonomic function and vagal tone, serving as a biomarker for both inflammatory state and taVNS efficacy.

Blood pressure monitors for assessing autonomic function and inflammation-related hemodynamic changes. Blood pressure variability analysis, similar to HRV, can provide autonomic information. Trends in mean arterial pressure, along with vasopressor requirements in critically ill patients, inform inflammation severity.

Temperature sensors for detecting fever (indicating inflammatory activation) or hypothermia (which may occur in severe inflammation with shock). Temperature variability and patterns of fever may guide treatment intensity.

Pulse oximetry for assessing oxygenation, with declining oxygen saturation potentially indicating inflammatory lung injury or other complications requiring intervention escalation.

Respiratory rate monitoring, with tachypnea indicating potential pulmonary complications or systemic inflammation.

Optionally, wearable or point-of-care devices for inflammatory biomarker measurement. While real-time continuous cytokine monitoring is not yet widely available, emerging technologies may facilitate more frequent measurement than traditional laboratory testing. Point-of-care CRP, IL-6, or other biomarkers measured every 4-12 hours could inform parameter adjustments.

Integration with hospital monitoring systems and electronic health records allows the taVNS device to receive data streams from standard monitoring equipment, laboratory results, and clinical assessments without requiring redundant sensors.

Controller with Adaptive Algorithm (2420): Data from physiologic sensors 2410 is processed by controller with adaptive algorithm 2420. The controller runs sophisticated algorithms that analyze HRV metrics including time-domain measures (SDNN, RMSSD), frequency-domain measures (LF power, HF power, LF/HF ratio), and nonlinear measures (sample entropy, detrended fluctuation analysis). Changes in HRV in response to taVNS and in the context of the inflammatory state inform parameter adjustments.

Blood pressure patterns and variability are analyzed for trends indicating improvement (stabilization, reduced vasopressor requirements) or deterioration (hypotension, increased vasopressor needs). Temperature trends are monitored, with fever patterns informing inflammatory activity. If available, inflammatory biomarker trends are integrated, with particular emphasis on IL-6 and CRP as key markers amenable to modulation by taVNS.

The algorithm assesses current inflammatory state by integrating multiple data sources. For example, rising IL-6 with worsening HRV and hemodynamic instability indicates escalating inflammation requiring parameter intensification. Declining IL-6 with improving HRV and hemodynamic stability indicates inflammation resolution, potentially allowing de-escalation. The algorithm also assesses adequacy of vagal activation by examining whether HRV is improving in response to taVNS, whether heart rate is decreasing to appropriate levels, and whether inflammatory biomarkers are responding. The algorithm includes safety monitoring for risk of adverse effects, particularly excessive bradycardia (heart rate <45-50 bpm), excessive hypotension in the absence of inflammation (which might indicate over-treatment), or other concerning patterns.

Memory Storage (2460): Memory storage 2460 maintains historical data on the patient's responses over hours to days of treatment. This historical data allows the algorithm to detect trends that may not be apparent from instantaneous measurements, to learn optimal parameter ranges for the individual patient (for example, identifying the minimum amplitude that produces HRV improvement, or the threshold above which side effects occur), to predict trajectories (is inflammation worsening, plateauing, or resolving?), and to provide data for clinical review and future research.

The bidirectional connection between controller 2420 and memory 2460 allows the algorithm to continuously update stored data and retrieve historical data to inform decisions.

Parameter Adjustment Logic (2430): Based on the analysis by the controller, parameter adjustment logic 2430 determines whether to modify stimulation parameters. Decision rules include the following.

If heart rate variability remains suppressed and inflammatory markers are elevated or rising, current amplitude is increased by 0.5 to 1.0 mA increments, up to the maximum safe or tolerable level, and/or duty cycle is increased, until a target of at least a threshold (e.g., 10%) increase in RMSSD or high-frequency HRV power is achieved relative to the pre-stimulation baseline measurement. This HRV titration target confirms adequate vagal activation and serves as the primary autonomic dosing endpoint. If the HRV target is achieved, current parameters are maintained. If the HRV target is exceeded substantially with concurrent hemodynamic stability, amplitude or duty cycle may be reduced to the minimum effective level.

If inflammatory biomarker levels are declining toward a target of at least a 20% reduction relative to the pre-treatment peak measurement particularly for interleukin-6 as the primary pharmacodynamic endpoint parameters may be de-escalated to reduce treatment intensity while maintaining biological effect. If inflammatory markers are not declining toward this threshold within 24 to 48 hours at current parameters, escalation to higher-intensity settings is indicated.

If excessive bradycardia occurs (heart rate below approximately 45 beats per minute sustained for more than 5 minutes), current amplitude is reduced or stimulation is temporarily paused until heart rate recovers, after which stimulation may be resumed at a lower amplitude.

If inflammatory markers spike acutely for example, interleukin-6 increasing by 50% or more within a 4-hour window the system escalates immediately to high-intensity protocol parameters without waiting for the next scheduled assessment interval.

If autonomic balance metrics, including LF/HF ratio, remain above a pre-specified threshold (for example, greater than 2.0), indicating persistent sympathetic dominance, frequency or duty cycle may be increased to augment parasympathetic activation.

More sophisticated implementations of the adjustment logic may employ machine learning approaches that identify patterns across multi-dimensional physiologic data streams to predict optimal parameters or risk of complications. Such algorithms require training on datasets from multiple patients and prospective validation before clinical deployment.

Output Circuitry (2440) and Electrodes (2450): The parameter adjustments determined by adjustment logic 2430 are implemented by output circuitry 2440, which modulates the electrical stimulation delivered via electrodes on auricular tissue 2450. The output circuitry can smoothly transition between parameter sets to avoid abrupt changes that might be uncomfortable or cause physiologic instability.

The stimulation creates physiologic changes in patient 2400, which are detected by sensors 2410, completing the feedback loop. This continuous cycle allows for dynamic optimization of therapy.

Technical Advantages of the Closed-loop adaptive systems herein may offer several advantages over open-loop (fixed parameter) protocols. They automatically compensate for inter-individual variability in response to taVNS, which can be substantial. Optimal parameters for one patient may be suboptimal or poorly tolerated by another. They provide real-time adaptation to changing inflammatory state, recognizing that inflammation is dynamic, particularly in the peri-intervention period. Parameters that are appropriate initially may become excessive as inflammation resolves, or may become insufficient if complications develop. They reduce clinician burden by automating parameter adjustments that would otherwise require frequent clinical assessment and manual adjustment. They potentially improve outcomes through continuous optimization that maintains parameters in the therapeutic window (sufficient to provide anti-inflammatory benefit, not excessive to cause side effects). They provide rich data for quality improvement and research through detailed logging of physiologic responses and parameter histories.

Challenges of closed-loop systems include greater complexity in device design and manufacturing, requiring sophisticated sensors, algorithms, and control systems. Regulatory pathways for closed-loop medical devices may be more demanding than for open-loop devices. Validation and safety testing must ensure that algorithms make appropriate decisions across the range of clinical scenarios. Cost may be higher than simpler devices. User acceptance and trust in autonomous systems may require education and demonstration of safety and efficacy.

Despite these challenges, closed-loop systems represent a logical evolution of taVNS technology, particularly for high-risk applications where optimization of therapy may have substantial clinical impact.

### Intervention-Specific Complication Profiles and Biomarker Patterns:

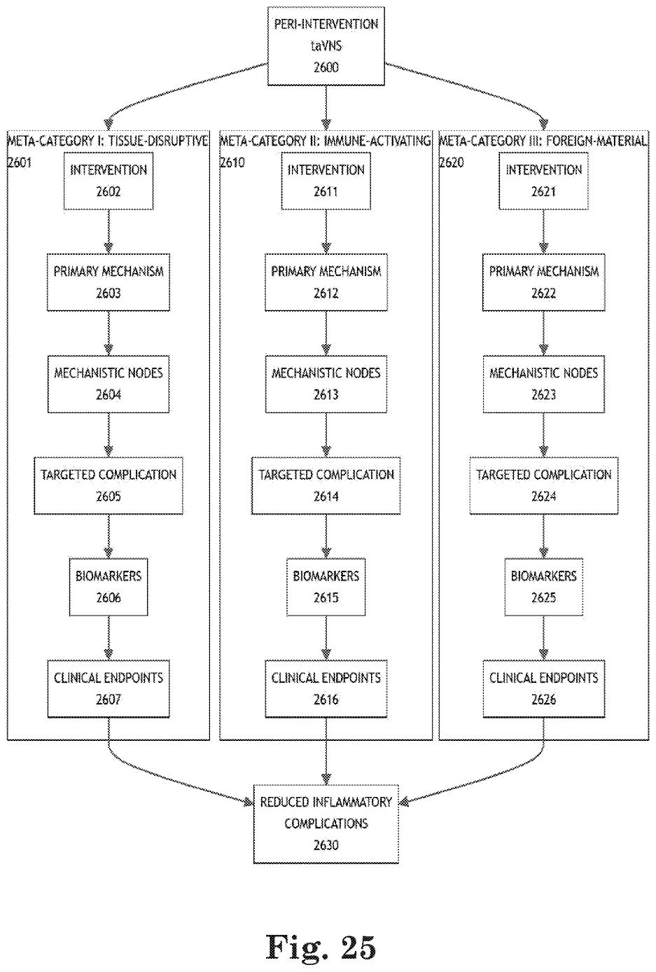

FIG. 25 illustrates the peri-intervention inflammatory modulation framework 2600 as three parallel application tracks corresponding to the three meta-categories of medical interventions, each proceeding through pre-intervention, intra-intervention, and post-intervention phases with category-specific protocol elements.

Meta-Category I Track (2601): Tissue-disruptive interventions (2602) including cranial surgery, spinal surgery, cardiothoracic surgery, cardiopulmonary bypass, radiation therapy, radiofrequency ablation, ischemia-reperfusion procedures, and organ transplantation are characterized by primary inflammatory mechanisms (2603) of tissue trauma, damage-associated molecular pattern (DAMP) release, complement activation, and ischemia-reperfusion injury. Mechanistic nodes (2604) targeted by taVNS in this track include cholinergic anti-inflammatory pathway activation through the α7 nAChR/NF-κB/JAK2-STAT3 molecular cascade, microglial M2 phenotype promotion, endothelial stabilization, and thromboinflammation modulation. Targeted complications (2605) include systemic inflammatory response syndrome, cerebral edema, acute kidney injury, and ischemia-reperfusion injury. Key monitored biomarkers (2606) include interleukin-6, C-reactive protein, high mobility group box 1 protein (HMGB1), neutrophil gelatinase-associated lipocalin (NGAL), glial fibrillary acidic protein (GFAP), S100B, von Willebrand factor (vWF), and D-dimer. Representative clinical endpoints (2607) include reduced systemic inflammatory response syndrome incidence, reduced cerebral edema, reduced acute kidney injury incidence, and reduced intensive care unit duration. For ischemia-reperfusion procedures, taVNS is synchronized with the reperfusion phase as described herein, with initiation or intensification of stimulation timed to begin just before or coinciding with restoration of blood flow.

Pre-intervention preconditioning (2601a) employs low to moderate intensity stimulation to prime neuroimmune anti-inflammatory pathways. Intra-intervention application (2601b) employs moderate to high intensity stimulation with timing coordinated to the peak inflammatory trigger, including reperfusion onset for ischemia-reperfusion procedures. Post-intervention treatment (2601c) employs high intensity stimulation in the early phase (first 24 to 72 hours), transitioning to moderate intensity as inflammation resolves, with duration guided by biomarker normalization including C-reactive protein returning toward baseline and interleukin-6 declining by at least 20% from peak.

Meta-Category II Track (2610): Immune-activating interventions (2611) including CAR-T cell therapy, immune checkpoint inhibitor therapy, stem cell infusion, viral vector gene therapy, and organ transplantation are characterized by primary inflammatory mechanisms (2612) of cytokine storm, T-cell hyperactivation, and complement-mediated immune toxicity. Mechanistic nodes (2613) targeted by taVNS in this track emphasize cholinergic anti-inflammatory pathway activation via α7 nAChR binding and NF-κB suppression as described with respect to FIG. 21, central high-gamma suppression in the insular cortex and orbitofrontal cortex as described with respect to FIG. 20, microglial M2 phenotype promotion, and HPA-axis modulation. Targeted complications (2614) include cytokine release syndrome, immune effector cell-associated neurotoxicity syndrome, immune-related adverse events, and graft-versus-host disease. Key monitored biomarkers (2615) include interleukin-6, ferritin, C-reactive protein, neurofilament light chain (NfL), tumor necrosis factor-α, GFAP, and C3a/C5a for viral vector applications. Representative clinical endpoints (2616) include reduced cytokine release syndrome grade, reduced immune effector cell-associated neurotoxicity syndrome score, reduced immune-related adverse events, and reduced graft-versus-host disease severity for hematopoietic stem cell transplantation applications, including monitoring of interleukin-6, tumor necrosis factor-α, and graft-versus-host disease severity score to assess the reduction in inflammatory complications.

For solid organ transplantation, alloimmune activation may produce elevated donor-specific antibodies (DSA) and interleukin-2 levels that predict acute rejection. Monitoring of DSA alongside taVNS delivery may guide post-transplant protocol duration and intensity, with elevated DSA levels supporting continuation of higher-intensity stimulation protocols.

Pre-intervention preconditioning (2610a) employs low to moderate intensity stimulation beginning one to seven days before infusion, with duration scaled to patient risk tier. Intra-intervention application (2610b) employs moderate to high intensity stimulation during infusion to reduce acute cytokine release. Post-intervention treatment (2610c) employs ongoing high intensity stimulation during the period of peak cytokine release risk, typically three to fourteen days following infusion, with parameter de-escalation guided by interleukin-6 trend and clinical stability, targeting at least a 20% reduction in interleukin-6 from pre-treatment peak as a pharmacodynamic de-escalation threshold.

Meta-Category III Track (2620): Foreign-material-introducing interventions (2621) including neurostimulator implantation, cardiac device implantation, orthopedic hardware placement, vascular stent placement, contrast agent administration, blood transfusion, and scaffold or biomaterial implantation are characterized by primary inflammatory mechanisms (2622) of foreign body response, complement activation, thromboinflammatory activation, and chronic fibrotic remodeling. Mechanistic nodes (2623) targeted by taVNS in this track emphasize cholinergic anti-inflammatory pathway activation, endothelial stabilization, thromboinflammation modulation, and macrophage M2 phenotype promotion to reduce fibrotic encapsulation. Targeted complications (2624) include acute device-related inflammation, fibrotic encapsulation, contrast-associated acute kidney injury, and transfusion reactions. For interventions involving implantation of scaffolds, bioengineered tissue constructs, or synthetic matrices where the inflammatory complication includes fibrotic encapsulation or impaired tissue integration, transforming growth factor-β (TGF-β) and matrix metalloproteinases (MMPs) are monitored as biomarkers of fibrotic remodeling. Key monitored biomarkers (2625) also include interleukin-6, high-sensitivity C-reactive protein, vWF, D-dimer, and angiopoietin-2. Representative clinical endpoints (2626) include reduced acute device-related inflammation, reduced fibrotic encapsulation, reduced contrast-associated acute kidney injury incidence, and reduced transfusion reaction severity.

Pre-intervention preconditioning (2620a) employs low to moderate intensity stimulation to reduce baseline inflammatory tone prior to material introduction. Intra-intervention application (2620b) employs moderate intensity stimulation timed to the period of maximum complement and thromboinflammatory activation. Post-intervention treatment (2620c) employs moderate intensity stimulation for the period of acute foreign body response, with extended low-intensity maintenance protocols for permanent implants to address chronic inflammatory remodeling, with heart rate variability titrated to achieve at least a 10% increase in RMSSD relative to pre-stimulation baseline as confirmation of adequate ongoing vagal activation.

The three parallel tracks converge at reduced inflammatory complications (2630), illustrating that while the specific protocol elements, monitored biomarkers, and clinical endpoints differ by intervention category, the fundamental mechanism of taVNS-mediated neuroimmune anti-inflammatory modulation and the peri-intervention timing framework apply across all three meta-categories.

Clinical Applications and Implementation: The methods, systems, and devices described herein can be applied across the spectrum of medical interventions that trigger inflammatory responses. The selection of specific protocols depends on multiple factors including the intervention category (tissue-disruptive, immune-activating, or foreign-material-introducing), patient risk stratification tier, baseline inflammatory markers, and/or the specific inflammatory complications most likely to occur.

### Device Integration and Implementation:

The taVNS devices described herein are adapted for peri-intervention use with the following considerations:

Adjustability for Comfort and Efficacy: The adjustable extending structures and anchor arms allow secure, comfortable placement that can remain in position for extended periods (hours to days) without causing pressure injury or discomfort. This is essential for multi-day preconditioning protocols and extended post-intervention treatment.

Battery Life and Rechargeability: For extended use, rechargeable batteries allow the device to operate continuously or for multiple sessions per day without replacement. Battery capacity sufficient for 24-48 hours of intermittent use (or 8-12 hours of continuous use) is desirable.

Programmability and Remote Control: The communication circuitry allows healthcare providers to remotely adjust parameters, check device status, and download usage/physiologic data without disturbing device placement. This is particularly valuable for patients under anesthesia or in intensive care settings.

Integration with Monitoring Systems: The monitoring circuitry can incorporate sensors for HRV, temperature, and potentially integration with hospital monitoring systems to receive data on blood pressure, inflammatory biomarkers, and/or other parameters for closed-loop control.

Biocompatibility and Sterility: For intra-operative use, the device must meet appropriate sterility standards. The electrodes and tissue-contacting surfaces may employ antimicrobial coatings or materials to reduce infection risk during extended use.

Each of the non-limiting examples herein can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described above. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of mitigating inflammatory complications associated with a medical intervention in a patient, the method comprising:
applying transcutaneous auricular vagal nerve stimulation to the patient at one or more times selected from before the medical intervention, during the medical intervention, and after the medical intervention, wherein the transcutaneous auricular vagal nerve stimulation is delivered via one or more stimulation elements positioned on an auricle of the patient; and
wherein the transcutaneous auricular vagal nerve stimulation activates one or more neuroimmune anti-inflammatory pathways to reduce inflammatory complications associated with the medical intervention.

2. The method of claim 1, further comprising:
applying a wearable device to an ear of the patient, the wearable device comprising a housing containing electronics and a power source, a first extending structure having a first end at the housing and a second end positioned within the ear when the wearable device is worn, an anchor arm extending laterally from the first extending structure, a second extending structure having a first end at the housing and a second end positioned within the ear when the wearable device is worn, and an adjustment mechanism configured to adjust a distance between the first extending structure and the second extending structure,
wherein the one or more stimulation elements are positioned on at least one of the first extending structure or the second extending structure.

3. The method of claim 1, wherein the transcutaneous auricular vagal nerve stimulation is delivered periodically in sessions having a duration in a range of about 30 seconds to about 24 hours.

4. The method of claim 1, further comprising: measuring at least one baseline inflammatory biomarker selected from C-reactive protein, interleukin-6, tumor necrosis factor-α, and/or ferritin prior to applying the transcutaneous auricular vagal nerve stimulation.

5. The method of claim 4, wherein the at least one baseline inflammatory biomarker further comprises at least one selected from high mobility group box 1 protein (HMGB1), glial fibrillary acidic protein (GFAP), S100B, neutrophil gelatinase-associated lipocalin (NGAL), von Willebrand factor, D-dimer, neurofilament light chain, calprotectin, C3a, C5a, angiopoietin-2, high-sensitivity C-reactive protein, neutrophil-to-lymphocyte ratio, interleukin-8, interleukin-2, donor-specific antibodies (DSA), lipopolysaccharide-binding protein (LBP), intercellular adhesion molecule-1 (ICAM-1), and/or vascular cell adhesion molecule-1 (VCAM-1).

6. The method of claim 1, further comprising: monitoring at least one physiologic parameter selected from heart rate variability, blood pressure variability, and/or body temperature during applying the transcutaneous auricular vagal nerve stimulation; and adjusting at least one stimulation parameter selected from current amplitude, pulse width, frequency, and/or duty cycle based on the monitored physiologic parameter.

7. The method of claim 6, wherein the adjusting at least one stimulation parameter comprises titrating the current amplitude to achieve at least a threshold increase in heart rate variability relative to a pre-stimulation baseline measurement.

8. The method of claim 1, further comprising: measuring at least one inflammatory biomarker selected from interleukin-6, tumor necrosis factor-α, C-reactive protein, and/or ferritin after applying the transcutaneous auricular vagal nerve stimulation; and comparing the measured inflammatory biomarker to a baseline measurement to assess reduction in inflammatory complications.

9. The method of claim 6, wherein the at least one stimulation parameter is adjusted to achieve at least a threshold amount of reduction in a measured inflammatory biomarker level relative to a pre-treatment peak measurement.

10. The method of claim 1, wherein the transcutaneous auricular vagal nerve stimulation is applied prior to the medical intervention as a preconditioning treatment for a duration in a range of hours to days before the medical intervention.

11. The method of claim 1, wherein the medical intervention comprises a tissue-disruptive intervention selected from the group consisting of: cranial surgery, spinal surgery, cardiothoracic surgery, cardiopulmonary bypass, radiation therapy, radiofrequency ablation, ischemia-reperfusion procedures, and organ transplantation.

12. The method of claim 11, wherein the ischemia-reperfusion procedure comprises a reperfusion phase, and wherein the transcutaneous auricular vagal nerve stimulation is synchronized with the reperfusion phase.

13. The method of claim 1, wherein the medical intervention comprises an immune-activating intervention selected from the group consisting of CAR-T cell therapy, immune checkpoint inhibitor therapy, stem cell infusion, viral vector gene therapy, and organ transplantation.

14. The method of claim 13, wherein the immune-activating intervention comprises hematopoietic stem cell transplantation or organ transplantation, and the inflammatory complication comprises graft-versus-host disease or acute rejection, and wherein at least one of interleukin-6, tumor necrosis factor-α, graft-versus-host disease severity score, and/or donor-specific antibodies (DSA) is monitored to assess the reduction in inflammatory complications and to guide post-transplant protocol duration and intensity.

15. The method of claim 1, wherein the medical intervention comprises a foreign-material-introducing intervention selected from the group consisting of: neurostimulator implantation, cardiac device implantation, orthopedic hardware placement, vascular stent placement, contrast agent administration, and blood transfusion.

16. The method of claim 15, wherein the foreign-material-introducing intervention comprises implantation of a scaffold, bioengineered tissue construct, or synthetic matrix, and the inflammatory complication comprises fibrotic encapsulation or impaired tissue integration, and wherein TGF-β and/or matrix metalloproteinases are monitored as biomarkers of fibrotic remodeling.

17. The method of claim 1, wherein the inflammatory complication is selected from the group consisting of: cytokine release syndrome, cerebral edema, systemic inflammatory response syndrome, acute kidney injury, immune effector cell-associated neurotoxicity syndrome, capillary leak syndrome, and ischemia-reperfusion injury.

18. The method of claim 1, wherein the one or more neuroimmune anti-inflammatory pathways are selected from the group consisting of: cholinergic anti-inflammatory pathway activation, central network suppression, reduced cerebral metabolism, autonomic stabilization, sympathetic withdrawal, endothelial stabilization, thromboinflammation modulation, HPA-axis modulation, microglial phenotype shifting, gut-immune axis modulation, and/or pain-inflammation feedback loop interruption.

19. The method of claim 18, wherein the cholinergic anti-inflammatory pathway activation comprises binding of acetylcholine to alpha-7 nicotinic acetylcholine receptors (α7 nAChR) on macrophages, microglia, or both, thereby suppressing nuclear factor-κB (NF-κB) activation through JAK2-STAT3 intracellular signaling and reducing production of pro-inflammatory cytokines.

20. The method of claim 1, wherein the transcutaneous auricular vagal nerve stimulation is delivered via electrodes as electrical stimulation at: a current amplitude in a range of 0.05 milliamps (mA) to 10 mA; a pulse width in a range of 50 microseconds to 2000 microseconds; and a frequency in a range of 0.1 Hertz (Hz) to 100 Hz.

21. The method of claim 1, wherein the transcutaneous auricular vagal nerve stimulation is adaptively modulated based on real-time physiologic feedback selected from heart rate variability, blood pressure variability, and/or measured cytokine levels.

22. The method of claim 1, wherein the transcutaneous auricular vagal nerve stimulation is delivered at a sub-perceptive current amplitude that suppresses high-gamma neural activity in the insular cortex, the orbitofrontal cortex, or both, to reduce centrally-mediated inflammatory drive through a central autonomic/limbic gateway mechanism that operates independently of conscious sensory perception and is active during general anesthesia or sedation.

23. The method of claim 1, wherein the stimulation is delivered via electrodes positioned within the external auditory canal, or via one or more percutaneous electrodes introduced through the skin of the auricle to a depth sufficient to reduce impedance and increase selectivity for activation of auricular branch of vagus nerve fibers relative to surface electrode delivery.

24. The method of claim 1, wherein the stimulation comprises a combination of transcutaneous auricular vagal nerve stimulation and at least one additional modality selected from transcutaneous cervical vagal nerve stimulation, intranasal vagal nerve stimulation delivered via electrodes positioned within the nasal cavity to access trigeminal-vagal convergent pathways, transcranial direct current stimulation, transcranial magnetic stimulation, pulsed electromagnetic field stimulation delivered via a micro-coil positioned over a cervical vagus nerve location, and/or low-intensity focused ultrasound.

25. The method of claim 1, wherein the transcutaneous auricular vagal nerve stimulation comprises a combination of electrical stimulation and at least one additional modality selected from vibratory stimulation at a frequency in a range of about 1 Hertz (Hz) to about 1000 Hz, thermal stimulation to a target tissue temperature in a range of about 10 degrees Celsius (° C.) to about 45° C., and/or low-intensity focused ultrasound at a frequency in a range of about 200 kilohertz (kHz) to about 5 megahertz (MHz), delivered simultaneously or in a time-staggered sequence.

26. The method of claim 1, wherein the transcutaneous auricular vagal nerve stimulation is delivered using a stochastically modulated waveform in which at least one stimulation parameter selected from current amplitude, pulse width, frequency, and/or inter-pulse interval is varied in a pseudo-random or noise-modulated manner within a therapeutically effective range, to reduce neural habituation and accommodation to repetitive stimulation during extended-duration protocols.

27. A transcutaneous auricular vagal nerve stimulation device configured for peri-intervention inflammatory modulation, comprising:
a housing adapted for placement relative to an ear of a patient;
one or more stimulation elements positioned to contact auricular tissue when the housing is placed relative to the ear;
a controller coupled to a memory storing instructions; and
output circuitry coupled to the controller and the one or more stimulation elements;
wherein the instructions, when executed by the controller, cause the output circuitry to deliver stimulation via the one or more stimulation elements according to a peri-intervention protocol selected based on an intervention category, wherein the intervention category is selected from tissue-disruptive intervention, immune-activating intervention, and/or foreign-material-introducing intervention.

28. The device of claim 27, wherein the instructions further cause the controller to adaptively adjust at least one stimulation parameter based on a measured physiologic parameter selected from heart rate variability, blood pressure variability, and/or temperature.

29. A non-transitory computer-readable medium storing instructions that, when executed by a controller of a transcutaneous auricular vagal nerve stimulation device, cause the device to:
receive at least one input selected from: (i) a medical intervention type, (ii) a patient risk stratification parameter, or (iii) both the medical intervention type and the patient risk stratification parameter;
select a peri-intervention stimulation protocol based on the at least one input; and
deliver stimulation via one or more stimulation elements positioned on an auricle of a patient according to the selected peri-intervention stimulation protocol to mitigate inflammatory complications associated with a medical intervention.

30. The non-transitory computer-readable medium of claim 29, wherein the instructions further cause the device to:
monitor a physiologic parameter selected from heart rate variability or blood pressure variability during delivery of the stimulation; and
adaptively adjust at least one stimulation parameter selected from current amplitude, pulse width, frequency, and/or duty cycle based on the monitored physiologic parameter.
